SEQUENCE LISTING (24 sequences)

SEQ ID NO: 1 - RNA, 20 nt, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
guuuaagggugaaauuuuuu

SEQ ID NO: 2 - RNA, 20 nt, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
aaaaauuucacccuuaaacu

SEQ ID NO: 3 - RNA, 20 nt, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
gccuaccugaggacaguaau

SEQ ID NO: 4 - RNA, 20 nt, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
uuacuguccucagguaggcu

SEQ ID NO: 5 - RNA, 20 nt, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
gaugugcacauaucaggaau

SEQ ID NO: 6 - RNA, 20 nt, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
uuccugauaugugcacaucu

SEQ ID NO: 7 - DNA, 21 bp, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
  misc_feature (21)..(21): deoxy T
guuuaagggugaaauuuuuut

SEQ ID NO: 8 - DNA, 21 bp, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
  misc_feature (20)..(21): phosphorothioate (PS) bond
  misc_feature (21)..(21): deoxy T
aaaaauuucacccuuaaacut

SEQ ID NO: 9 - DNA, 21 bp, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
  misc_feature (20)..(21): phosphorothioate (PS) bond
  misc_feature (21)..(21): deoxy T
gccuaccugaggacaguaaut

SEQ ID NO: 10 - DNA, 21 bp, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
  misc_feature (20)..(21): phosphorothioate (PS) bond
  misc_feature (21)..(21): deoxy T
uuacuguccucagguaggcut

SEQ ID NO: 11 - DNA, 21 bp, Artificial Sequence
  Synthetic Construct
  misc_feature (1)..(1): unlocked nucleic acid base
  misc_feature (1)..(2): phosphorothioate (PS) bond
  misc_feature (2)..(2): 2′-O-methyl modified ribonucleotide
  misc_feature (4)..(4): 2′-O-methyl modified ribonucleotide
  misc_feature (6)..(6): 2′-O-methyl modified ribonucleotide
  misc_feature (8)..(8): 2′-O-methyl modified ribonucleotide
  misc_feature (12)..(12): 2′-O-methyl modified ribonucleotide
  misc_feature (14)..(14): 2′-O-methyl modified ribonucleotide
  misc_feature (16)..(16): 2′-O-methyl modified ribonucleotide
  misc_feature (18)..(18): 2′-O-methyl modified ribonucleotide
  misc_feature (19)..(20): phosphorothioate (PS) bond
  misc_feature (20)..(20): unlocked nucleic acid base
  misc_feature (20)..(21): phosphorothioate (PS) bond
  misc_feature (21)..(21): deoxy T
gaugugcacauaucaggaaut

SEQ ID NO: 12 - DNA, 21 bp, Artificial Sequence
  Synthetic Construct
  misc_feature (1)..(1): 2′-O-methyl modified ribonucleotide
  misc_feature (3)..(3): 2′-O-methyl modified ribonucleotide
  misc_feature (5)..(5): 2′-O-methyl modified ribonucleotide
  misc_feature (7)..(7): 2′-O-methyl modified ribonucleotide
  misc_feature (9)..(9): 2′-O-methyl modified ribonucleotide
  misc_feature (11)..(13): 2′-O-methyl modified ribonucleotide
  misc_feature (15)..(15): 2′-O-methyl modified ribonucleotide
  misc_feature (17)..(17): 2′-O-methyl modified ribonucleotide
  misc_feature (19)..(19): 2′-O-methyl modified ribonucleotide
  misc_feature (20)..(20): unlocked nucleic acid base
  misc_feature (20)..(21): phosphorothioate (PS) bond
  misc_feature (21)..(21): deoxy T
uuccugauaugugcacaucut

SEQ ID NO: 13 - RNA, 19 nt, Artificial Sequence
  Synthetic Construct
guuuaagggugaaauuuuu

SEQ ID NO: 14 - RNA, 19 nt, Artificial Sequence
  Synthetic Construct
aaaaauuucacccuuaaac

SEQ ID NO: 15 - RNA, 19 nt, Artificial Sequence
  Synthetic Construct
gccuaccugaggacaguaa

SEQ ID NO: 16 - RNA, 19 nt, Artificial Sequence
  Synthetic Construct
uuacuguccucagguaggc

SEQ ID NO: 17 - RNA, 19 nt, Artificial Sequence
  Synthetic Construct
gaugugcacauaucaggaa

SEQ ID NO: 18 - RNA, 19 nt, Artificial Sequence
  Synthetic Construct
uuccugauaugugcacauc

SEQ ID NO: 19 - DNA, 21 bp, Artificial Sequence
  Synthetic Construct
guuuaagggugaaauuuuutt

SEQ ID NO: 20 - DNA, 21 bp, Artificial Sequence
  Synthetic Construct
aaaaauuucacccuuaaacat

SEQ ID NO: 21 - DNA, 21 bp, Artificial Sequence
  Synthetic Construct
gccuaccugaggacaguaatt

SEQ ID NO: 22 - DNA, 21 bp, Artificial Sequence
  Synthetic Construct
uuacuguccucagguaggctc

SEQ ID NO: 23 - DNA, 21 bp, Artificial Sequence
  Synthetic Construct
gaugugcacauaucaggaatt

SEQ ID NO: 24 - RNA, 21 nt, Artificial Sequence
  Synthetic Construct
uuccugauaugugcacaucca

(19) United States
(12) Patent Application Publication
(10) Pub. No.: US 2024/0309366 A1
(43) Pub. Date: Sep. 19, 2024

(54) COMPOSITIONS AND METHODS FOR TREATING METABOLIC DISORDERS

(71) Applicants: Arcturus Therapeutics, Inc., San Diego, CA (US); CONSORCIO CENTRO DE INVESTIGACIÓN BIOMÉDICA EN RED, M.P., Madrid (ES); INSTITUT D’INVESTIGACIÓ BIOMÉDICA DE GIRONA DR JOSEP TRUETA, Salt (ES); UNIVERSITAT DE BARCELONA, Barcelona (ES)

(72) Inventors: Ramón Díaz TRELLES, San Diego, CA (US); Kiyoshi TACHIKAWA, San Diego, CA (US); Priya Prakash KARMALI, San Diego, CA (US); Rajesh MUKTHAVARAM, San Diego, CA (US); Padmanabh CHIVUKULA, San Diego, CA (US); José María MORENO-NAVARRETE, Madrid (ES); Aleix Gavaldá NAVARRO, Madrid (ES); José Manuel FERNÁNDEZ-REAL, Salt (ES); Marta Giralt OMS, Barcelona (ES); Francesc Villarroya GOMBAU, Barcelona (ES)

(21) Appl. No.: 18/260,096

(22) PCT Filed: Dec. 30, 2021
(86) PCT No.: PCT/US21/65757

§ 371 Date: Jun. 30, 2023

Related U.S. Application Data
(60) Provisional application No. 63/132,881, filed on Dec. 31, 2020.

(51) Int. Cl. C12N 15/113; A61K 9/51; A61K 31/713; A61P 3/04

(52) U.S. Cl. CPC C12N 15/113; A61K 9/5146; A61K 31/713; A61P 3/04

(57) ABSTRACT

Provided herein are compositions and methods for treating metabolic disorders, such as hepatic steatosis, adipose tissue dysfunction, and insulin resistance. Small interfering RNAs (siRNAs) targeting LPS-binding protein (LBP) that include unlocked nucleotides (UNA) and their therapeutic applications for the treatment of metabolic disorders are provided herein.

### CROSS-REFERENCES TO RELATED APPLICATIONS

This application claims the benefit of U.S. Provisional Application No. 63/132,881, filed Dec. 31, 2020, which is incorporated herein by reference in its entirety and for all purposes.

### TECHNICAL FIELD

The present disclosure relates generally to the treatment of metabolic disorders and conditions and more specifically to inhibiting expression of lipopolysaccharide binding protein (LBP) using RNAi targeting the liver.

### SEQUENCE LISTING

The instant application contains a Sequence Listing which has been submitted electronically in ASCII format and is hereby incorporated by reference in its entirety. Said ASCII copy, created on Dec. 29, 2021 is named 049386_535001WO_SL_ST25.txt and is 17.6 kilobytes in size.

### BACKGROUND

Obesity, characterized by adipose tissue enlargement and increased body fat accretion, is a worldwide epidemic caused by disturbed energy balance such as increased food energy intake and/or decreased energy expenditure. Obesity is the most important factor in the progression of metabolic diseases, including metabolic dysfunction-associated fatty liver disease (MAFLD; also known as nonalcoholic fatty liver disease or NAFLD), type 2 diabetes, dyslipidemia, sleep apnea, kidney disease, arterial hypertension, ischemic heart disease, osteoarthritis, and some types of cancer, contributing to major disease burden worldwide. Metabolic diseases such as MALFD further contribute to the severity of other obesity-associated metabolic disturbances that share a common pathology, such as insulin resistance, dyslipidemia, and type 2 diabetes.

Lipopolysaccharide-binding protein (LBP) has been identified as a component of innate immunity. The liver is the main source of circulating LBP, and LBP is also present in adipose tissue. Although circulating LBP has been implicated in development of obesity, insulin resistance, metabolic syndrome, fat accumulation, and fibrosis, the role of LBP in obesity-associated adipose tissue dysfunction and the role of liver LBP in obesity-associated fatty liver disease are not known. Moreover, very few approved pharmacological therapies exist that are specific for MALFD, and few therapeutic targets have been identified for the prevention of weight and fat mass gain and obesity-associated fat accretion. Thus, there exists a need for new therapeutics for the treatment and prevention of obesity and obesity-associated metabolic disorders.

### SUMMARY

The present disclosure is based on the seminal discovery of LBP as a therapeutic target for the treatment of obesity, obesity-related metabolic disorders, and obesity-related tissue dysfunction. Accordingly, the present disclosure provides compositions and methods for the treatment and prevention of obesity, obesity-related metabolic disorders such as liver steatosis, and adipose tissue dysfunction by targeting liver LBP gene expression.

Provided herein, in some embodiments, are compounds for RNA interference that include: (A)(i) a sense strand comprising a sequence having at least 80% identity to a sequence of 5′-GUUUAAGGGUGAAAUUUUU-3′ (SEQ ID NO:13), and (ii) an antisense strand comprising a sequence having at least 80% identity to a sequence of 5′-AAAAAUUUCACCCUUAAAC-3′ (SEQ ID NO:14); (B)(i) a sense strand comprising a sequence having at least 80% identity to a sequence of 5′-GCCUACCUGAGGACAGUAA-3′ (SEQ ID NO:15), and (ii) an antisense strand comprising a sequence having at least 80% identity to a sequence of 5′-UUACUGUCCUCAGGUAGGC-3′ (SEQ ID NO:16); or (C)(i) a sense strand comprising a sequence having at least 80% identity to a sequence of 5′-GAUGUGCACAUAUCAGGAA-3′ (SEQ ID NO:17), and (ii) an antisense strand comprising a sequence having at least 80% identity to a sequence of 5′-UUCCUGAUAUGUGCACAUC-3′ (SEQ ID NO:18); wherein the compound comprises at least one UNA monomer and at least one chemically modified monomer.

In one aspect, G at a first position from the 5′ end of the sense strand is replaced by an UNA monomer. In another aspect, the UNA monomer at the first position from the 5′ end of the sense strand is UNA-G, UNA-U, UNA-A, or UNA-C. In one aspect, the UNA monomer at the first position from the 5′ end of the sense strand is UNA-G. In another aspect, the UNA monomer at the first position from the 5′ end of the sense strand is connected to a 5′ unmodified monomer or a 5′ chemically modified monomer by a phosphorothioate linkage, a chiral phosphorothioate linkage, or a phosphorodithioate linkage.

In one aspect, the sense strand, the antisense strand, or both the sense strand and the antisense strand comprise an UNA monomer added at the 3′end. In another aspect, the UNA monomer added at the 3′ end is UNA-U, UNA-G, UNA-A, UNA-C, or any combination thereof. In yet another aspect, the UNA monomer added at the 3′ end is UNA-U. In an aspect, the UNA monomer added at the 3′ end is connected to a 3′ unmodified monomer or a 3′ chemically modified monomer by a phosphorothioate linkage, a chiral phosphorothioate linkage, or a phosphorodithioate linkage.

In one aspect, the sense strand, the antisense strand, or both the sense strand and the antisense strand of compounds provided herein include at least one chemically modified monomer. In another aspect, the sense strand, the antisense strand, or both the sense strand and the antisense strand include 1 to 19 chemically modified monomers. In yet another aspect, the at least one chemically modified monomer is a methylated monomer. In a further aspect, the at least one chemically modified monomer is a 2′-O-methyl modified monomer, an inverted thymidine monomer, an L-thymidine monomer, a glyceryl nucleotide, a 2′-methoxyethoxy nucleotide, a 2′-deoxy-2′-fluoro ribonucleotide, or any combination thereof. In yet a further aspect, compounds provided herein include a deoxy T monomer connected to the UNA monomer added at the 3′end of the sense strand, the antisense strand, or both the sense strand and the antisense strand. In one aspect, the deoxy T monomer is connected to the UNA monomer added at the 3′end of the sense strand, the antisense strand, or both the sense strand and the antisense strand by a phosphorothioate linkage, a chiral phosphorothioate linkage, or a phosphorodithioate linkage.

Provided herein, in some embodiments, are compounds for RNA interference comprising: (A)(i) a sense strand comprising a sequence having at least 80% identity to a sequence of SEQ ID NO:1, and (ii) an antisense strand comprising a sequence having at least 80% identity to a sequence of SEQ ID NO:2; (B)(i) a sense strand comprising a sequence having at least 80% identity to a sequence of SEQ ID NO:3, and (ii) an antisense strand comprising a sequence having at least 80% identity to a sequence of SEQ ID NO:4; or (C)(i) a sense strand comprising a sequence having at least 80% identity to a sequence of SEQ ID NO:5, and (ii) an antisense strand comprising a sequence having at least 80% identity to a sequence of SEQ ID NO:6. In one aspect, compounds provided herein further include a deoxy T monomer at the 3′ end of the sense strand, the antisense strand, or both the sense strand and the antisense strand. In another aspect, the deoxy T monomer is linked to the 3′ end of the sense strand, the antisense strand, or both the sense strand and the antisense strand by a phosphorothioate linkage, a chiral phosphorothioate linkage, or a phosphorodithioate linkage.

Provided herein, in some embodiments, are compounds for RNA interference comprising: (A)(i) a sense strand comprising a sequence having at least 80% identity to a sequence of SEQ TD NO:7, and (ii) an antisense strand comprising a sequence having at least 80% identity to a sequence of SEQ TD NO:8; (B)(i) a sense strand comprising a sequence having at least 80% identity to a sequence of SEQ TD NO:9, and (ii) an antisense strand comprising a sequence having at least 80% identity to a sequence of SEQ TD NO:10; or (C)(i) a sense strand comprising a sequence having at least 80% identity to a sequence of SEQ TD NO:11, and (ii) an antisense strand comprising a sequence having at least 80% identity to a sequence of SEQ ID NO:12.

In one aspect, compounds provided herein selectively inhibit lipopolysaccharide binding protein (LBP) expression.

Provided herein, in some embodiments, are pharmaceutical compositions that include a compound provided herein and a pharmaceutically acceptable carrier. In some aspects, the carrier comprises a transfection reagent, a nanoparticle, or a liposome. In some aspects, pharmaceutical compositions provided herein comprise a cationic lipid, a helper lipid, a cholesterol, and a PEG-lipid conjugate. In some aspects, pharmaceutical compositions provided herein comprise lipid nanoparticles. In some aspects, the lipid nanoparticles have a percent encapsulation of at least about 50%. In some aspects, the lipid nanoparticles have a size less than about 100 nm. In some aspects, the lipid nanoparticles have an average particles size of between about 50 and about 85 nm. In some aspects, the cationic lipid included in pharmaceutical compositions provided herein is an ionizable cationic lipid. In further aspects, the ionizable cationic lipid has a structure selected from:

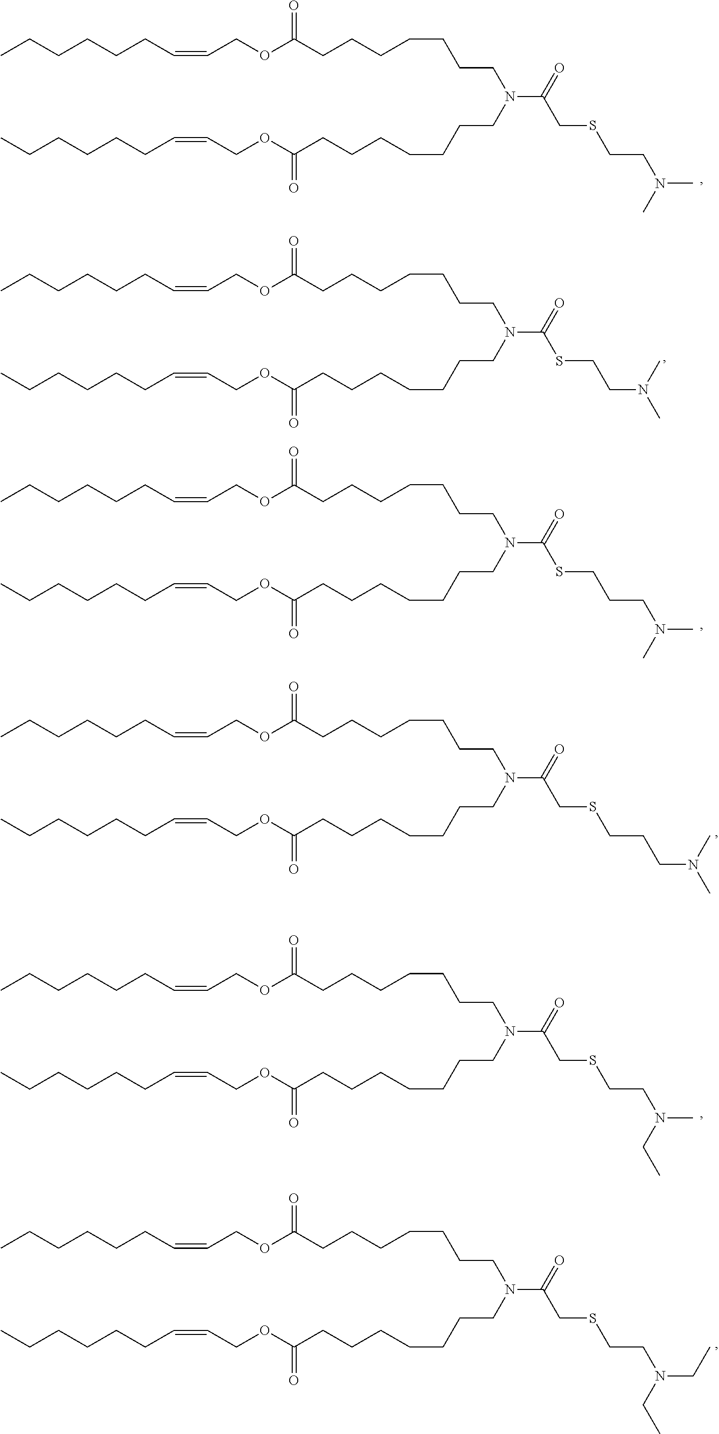

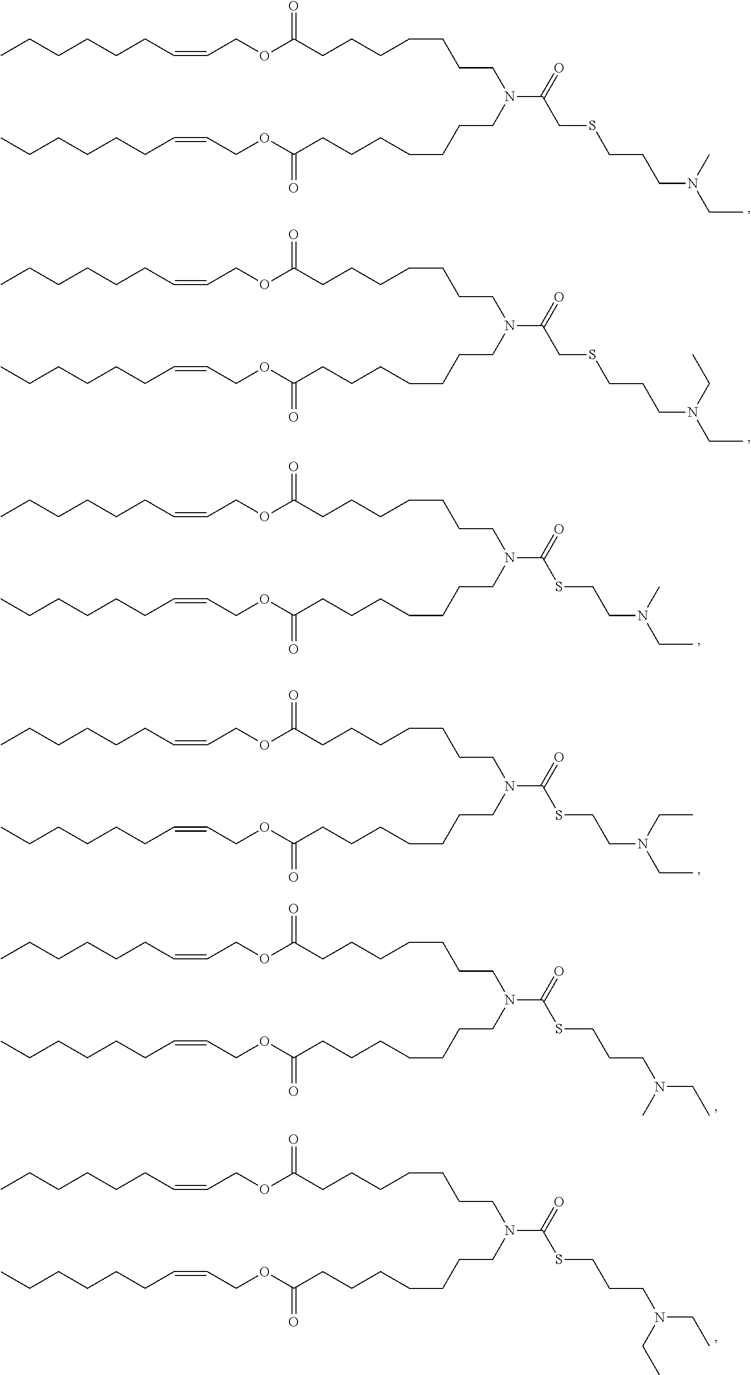

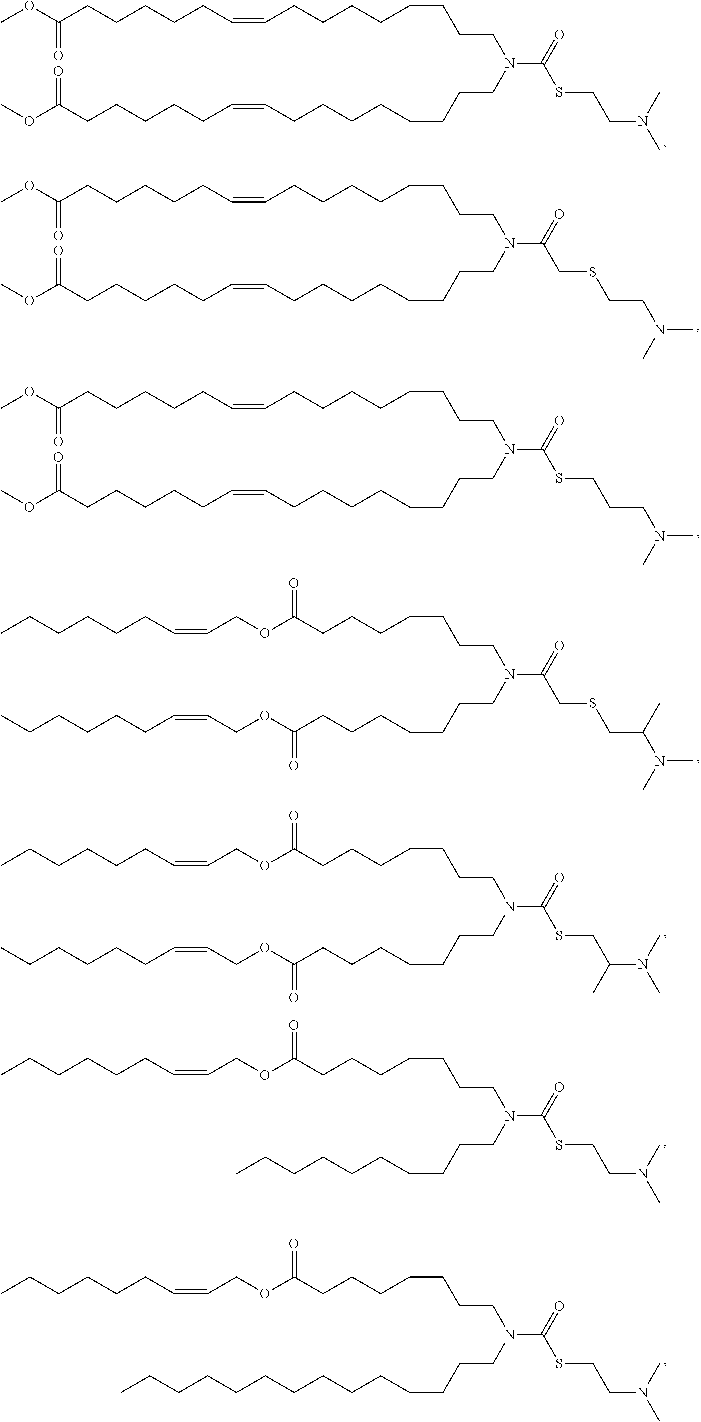

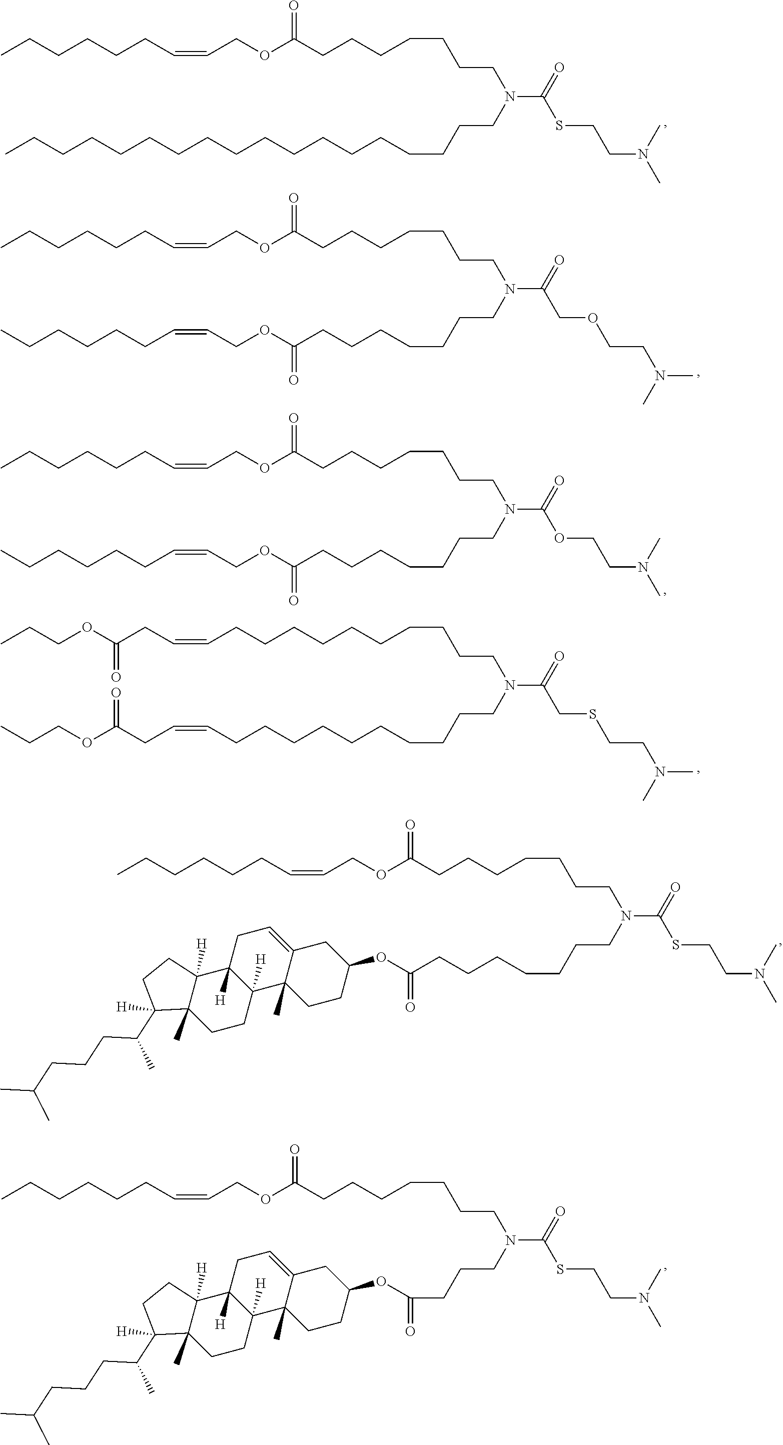

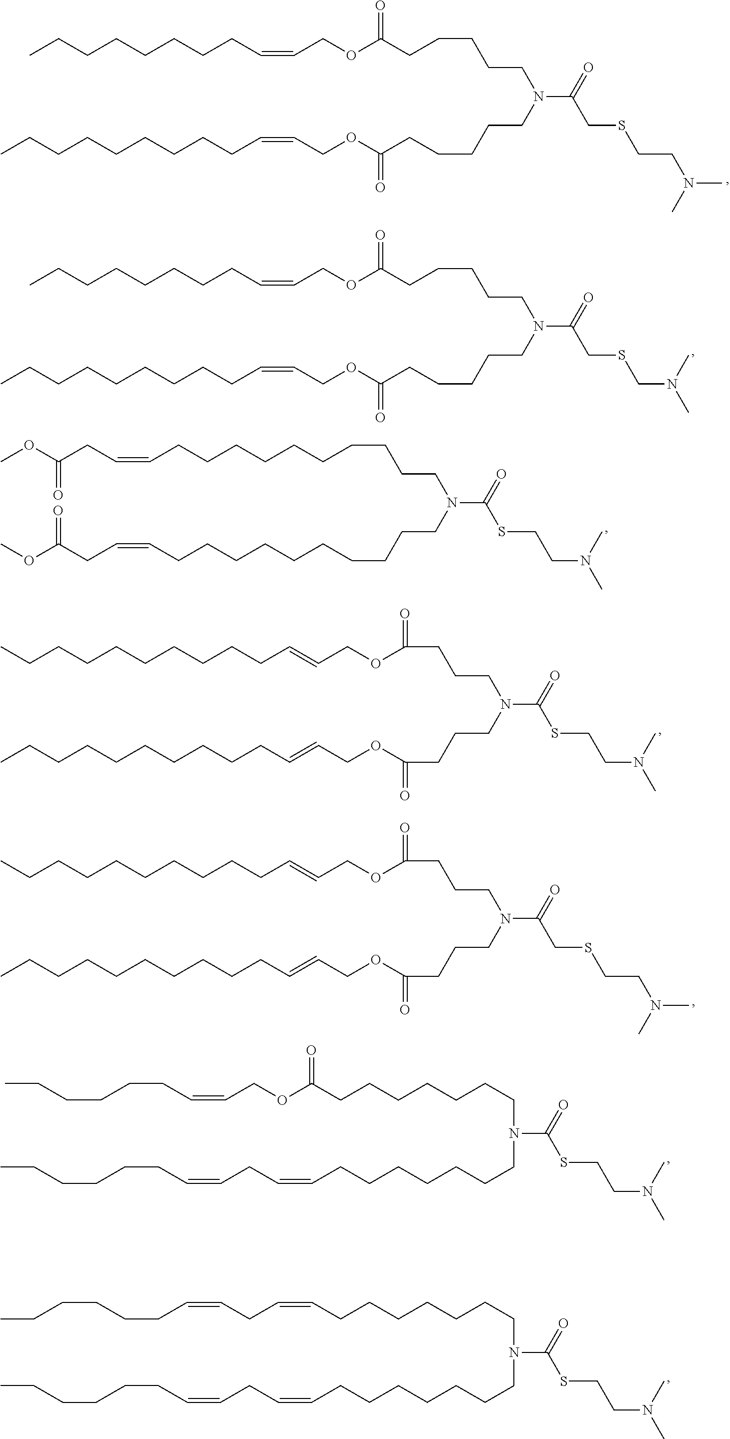

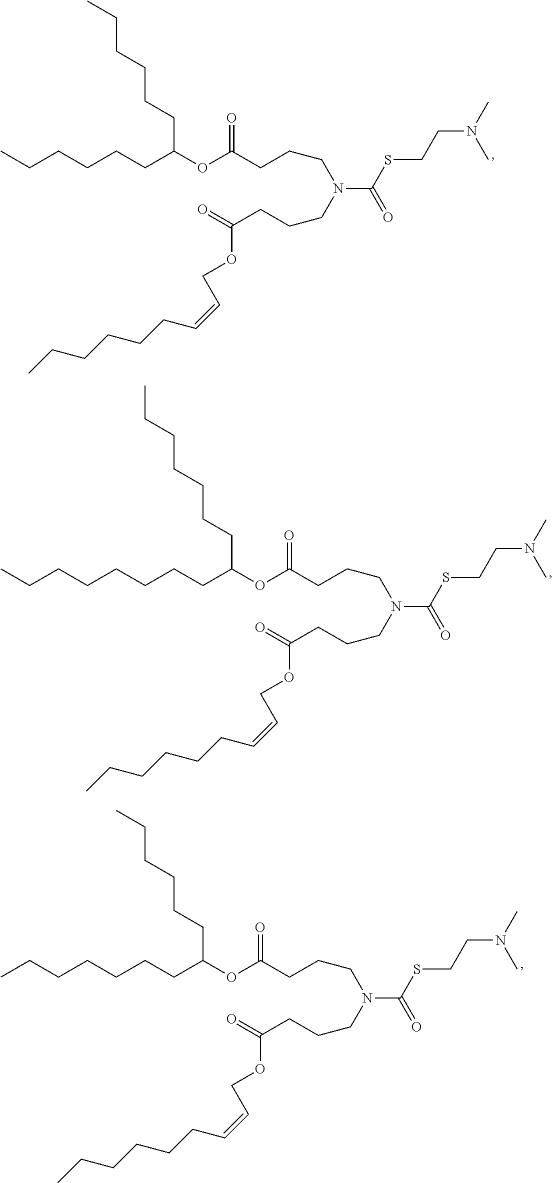

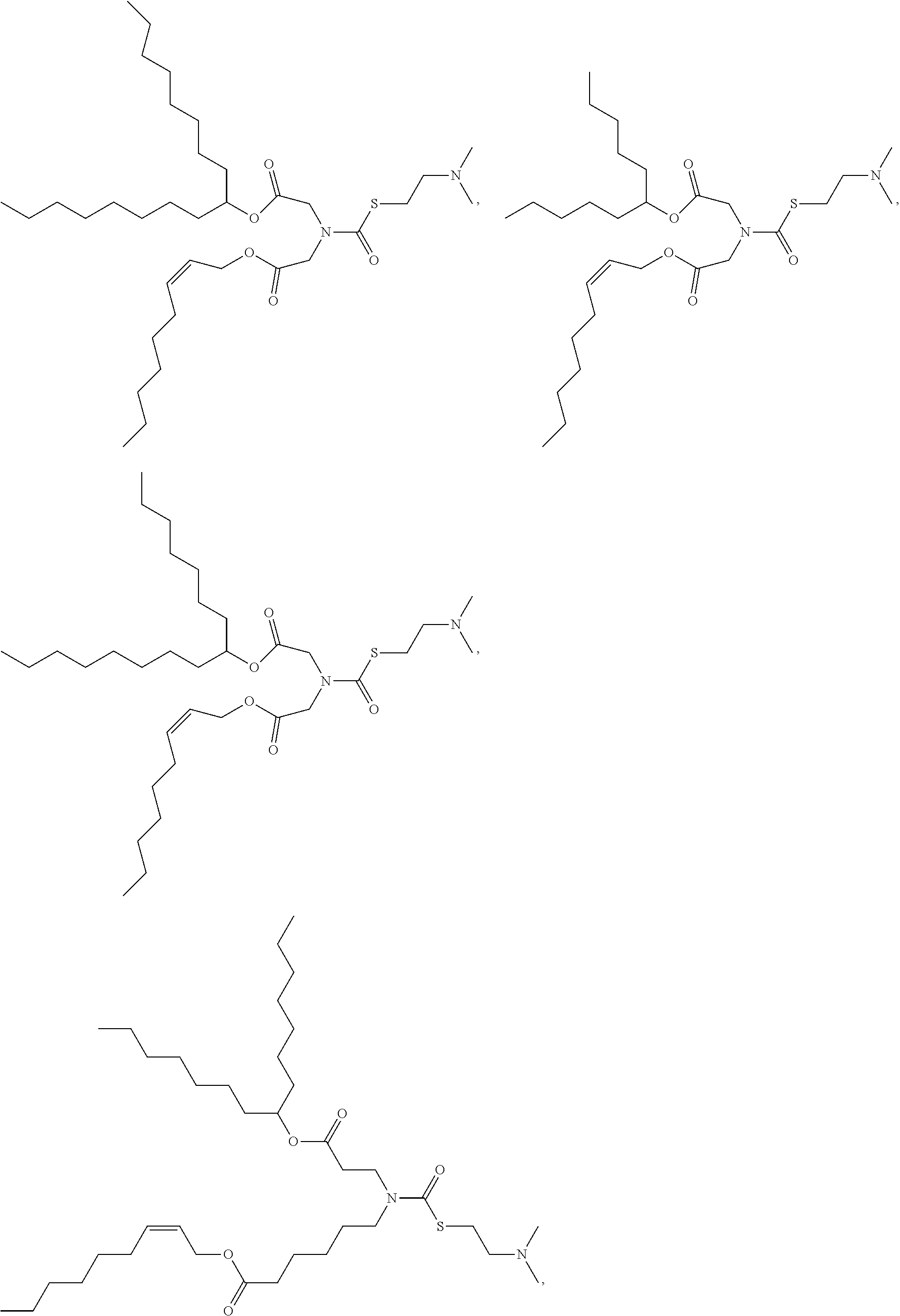

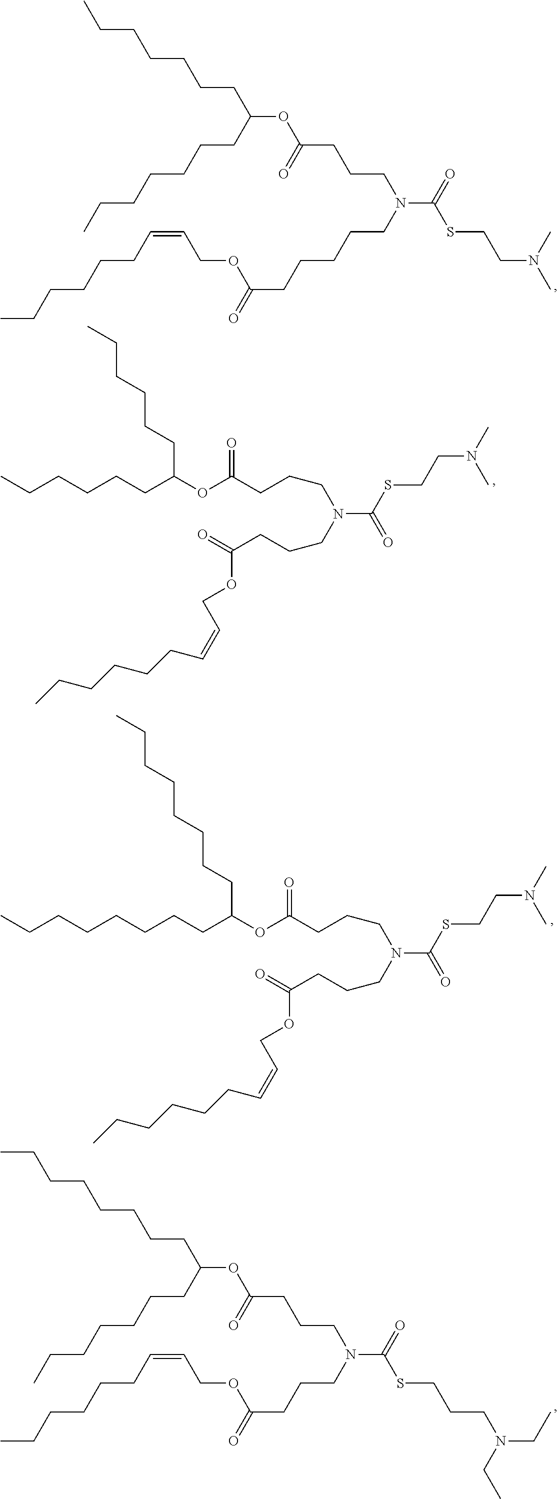

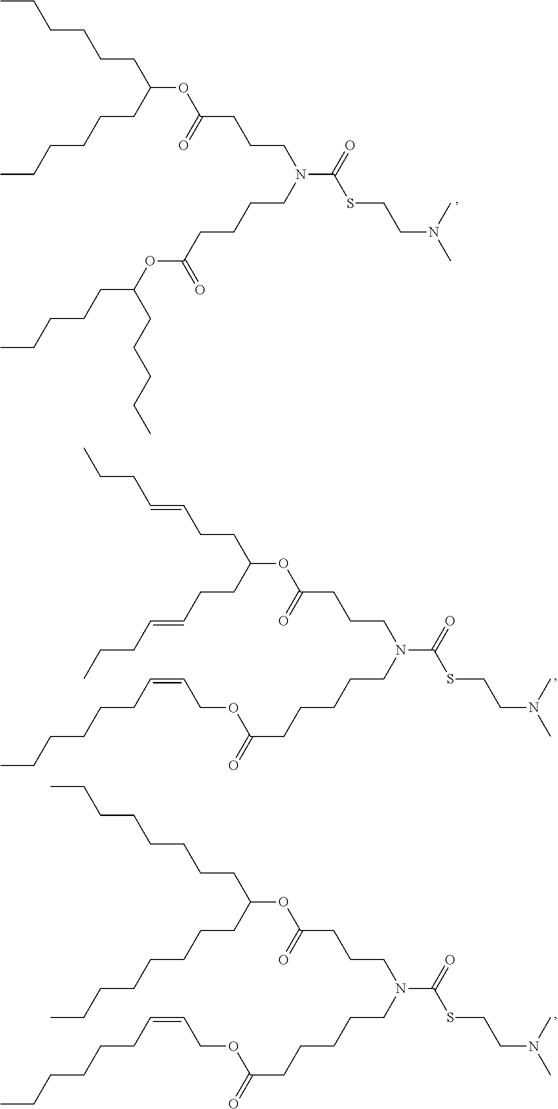

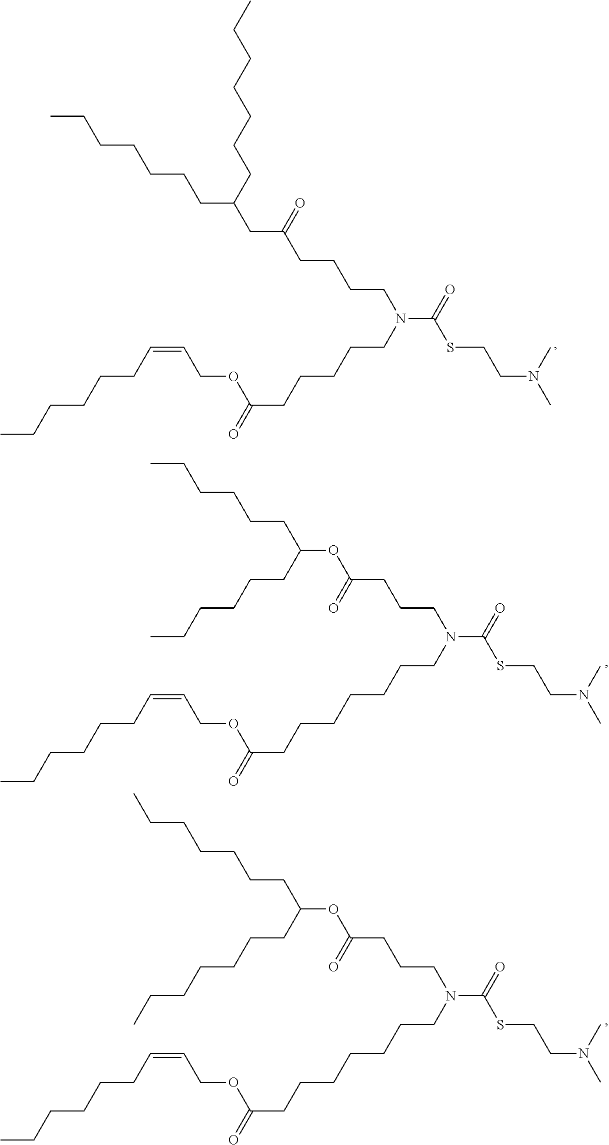

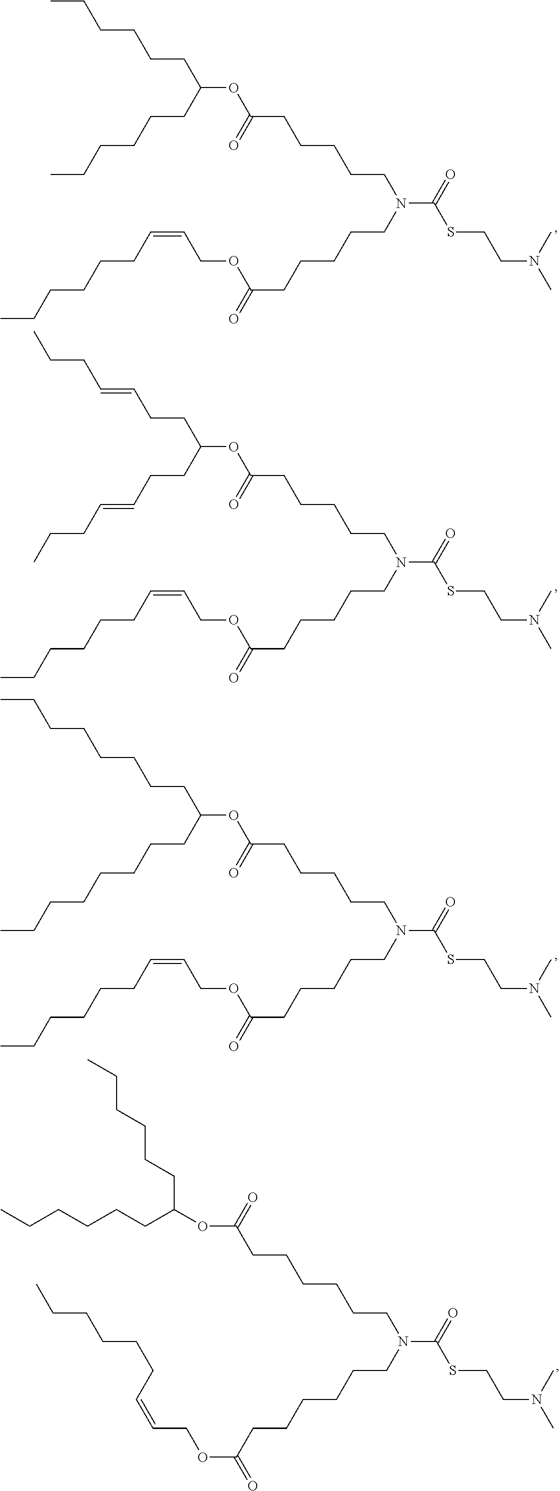

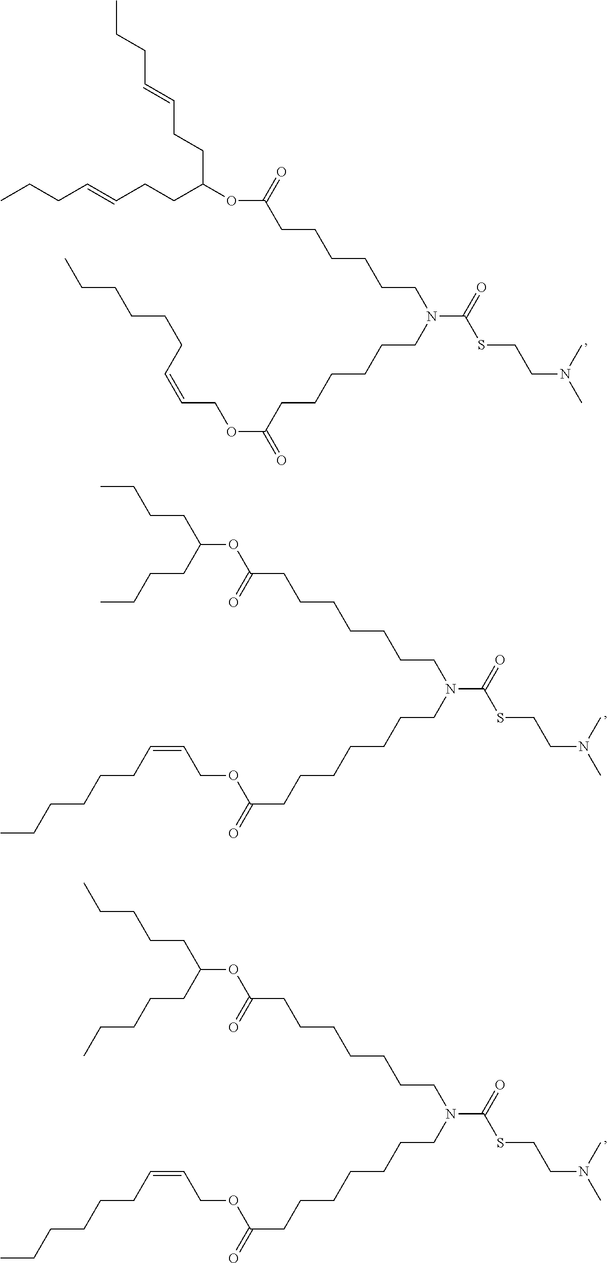

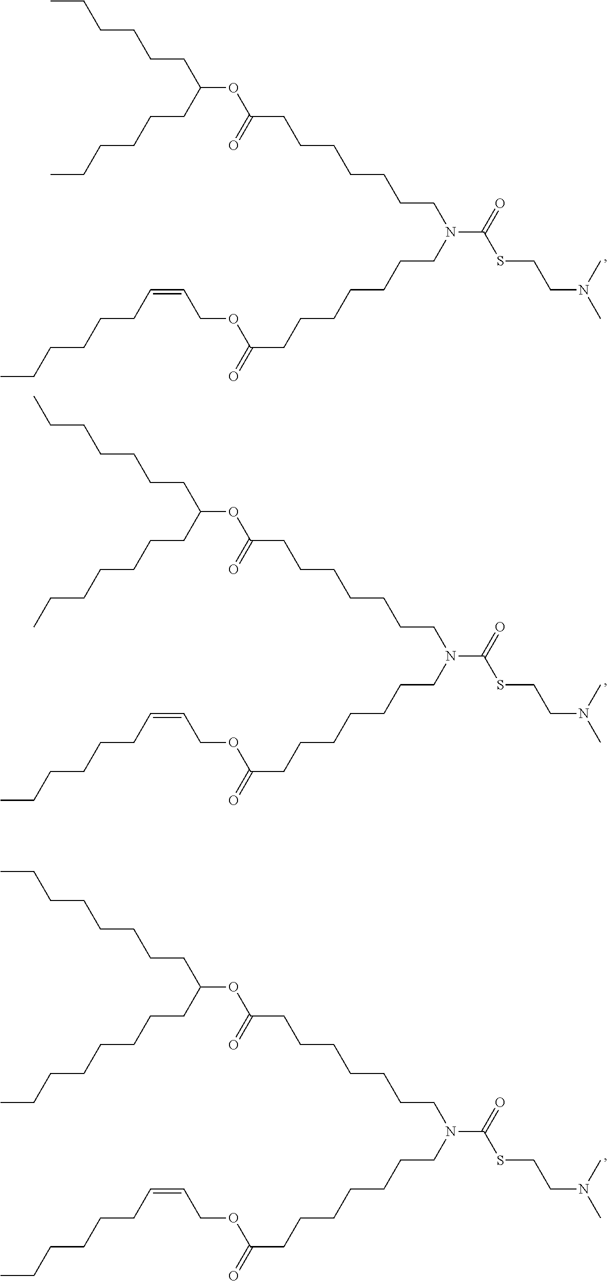

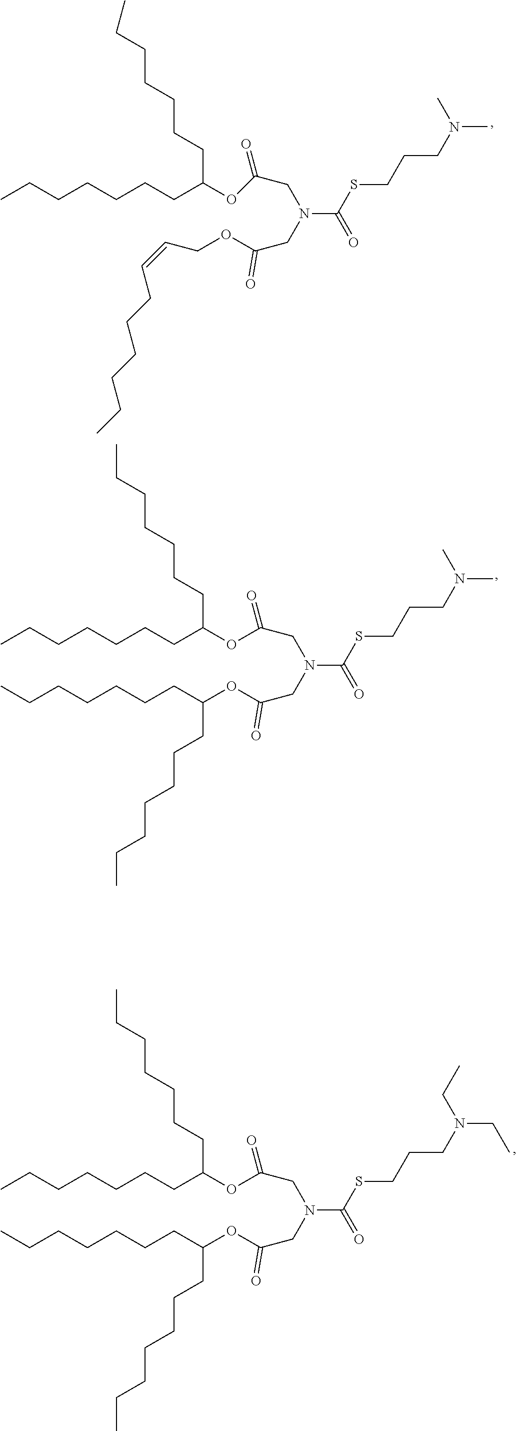

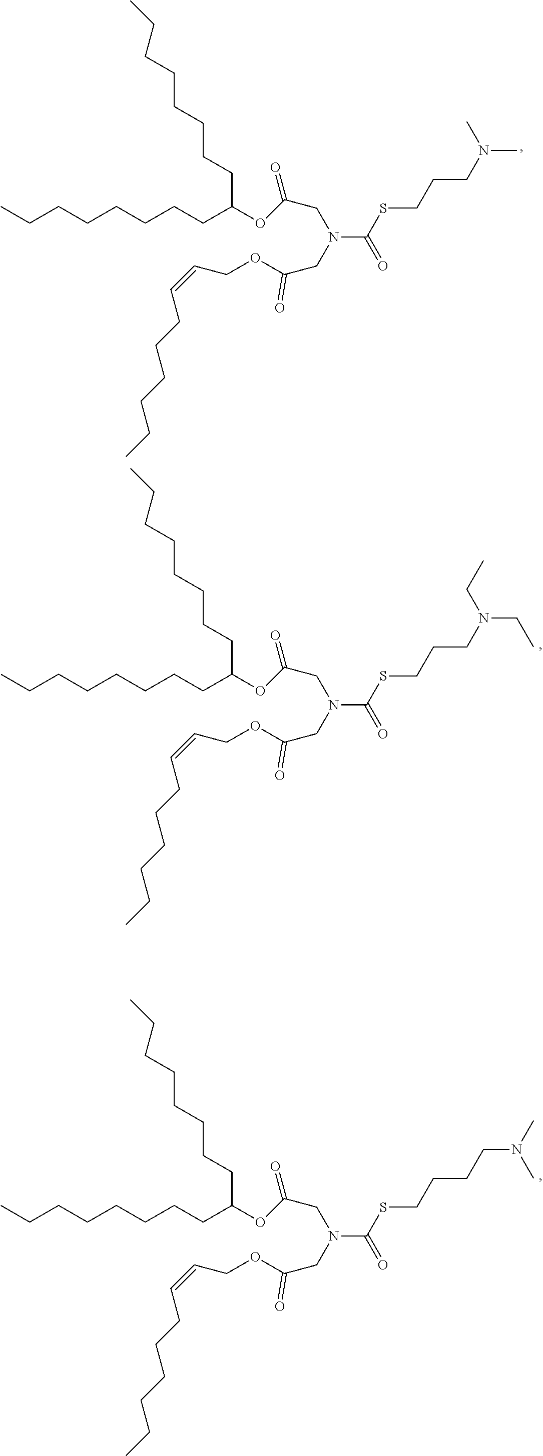

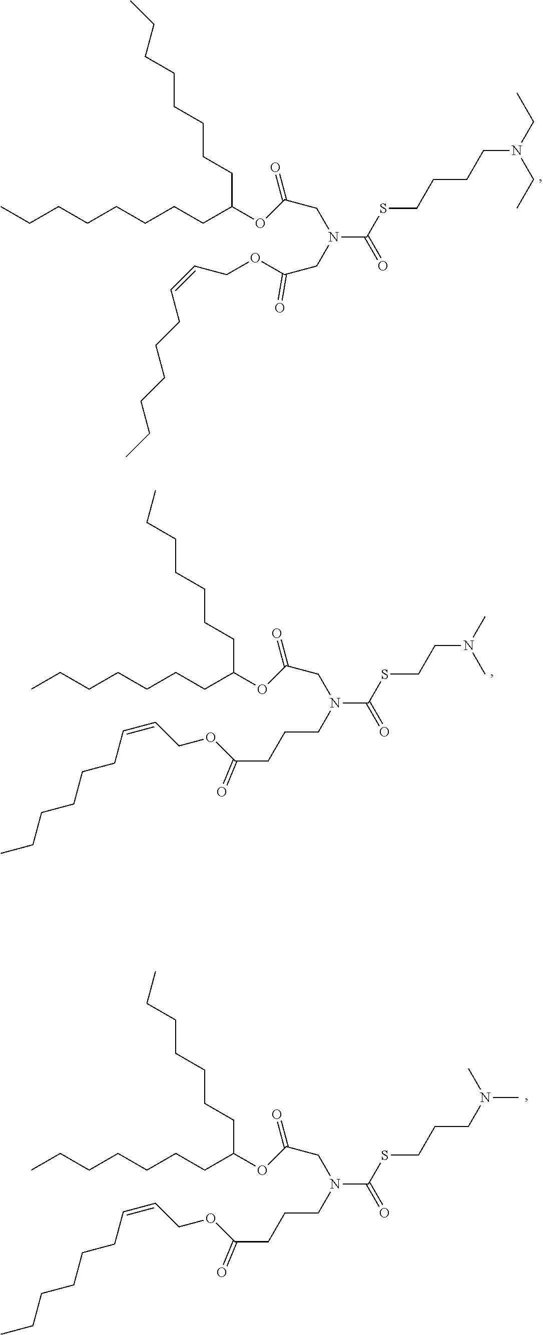

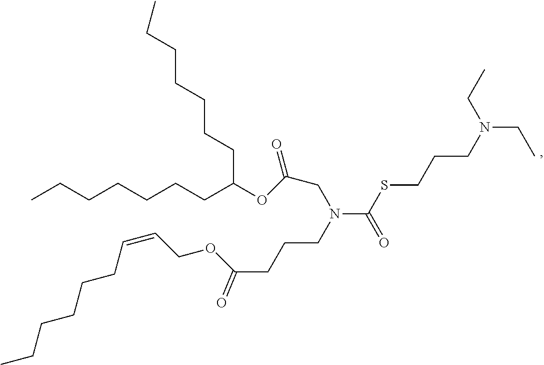 [SMILES: CCCCCC/C=C\COC(=O)CCCN(CC(=O)OC(CCCCCCC)CCCCCCC)C(=O)SCCCN(CC)CC]

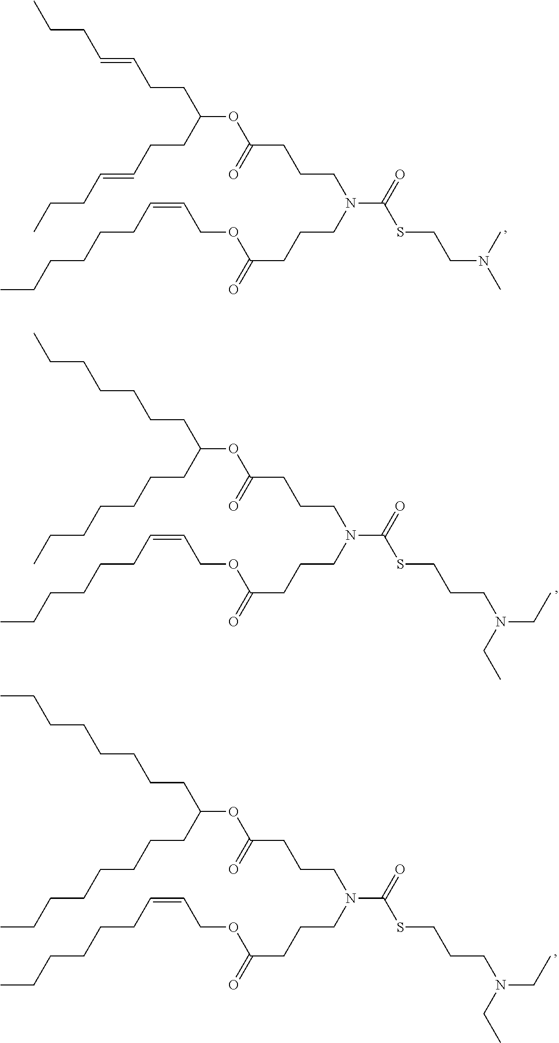

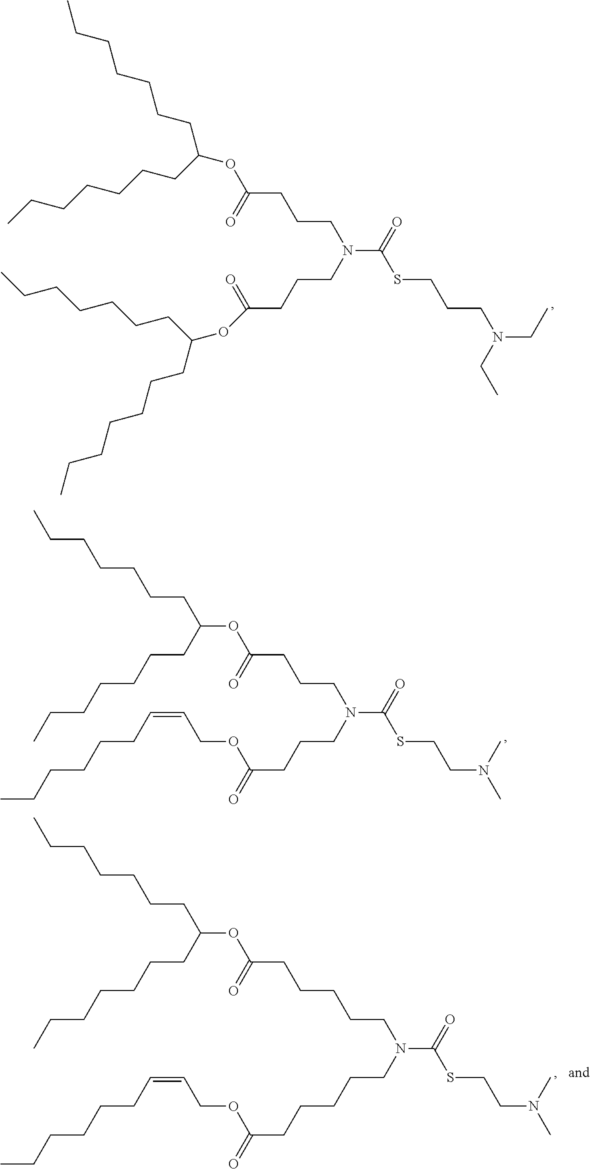

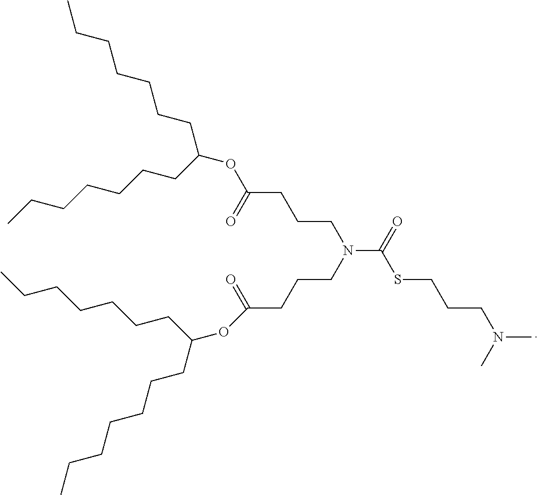 [SMILES: CCCCCCCC(CCCCCCC)OC(=O)CCCN(CCCC(=O)OC(CCCCCCC)CCCCCCC)C(=O)SCCCN(C)C]

In some aspects, the helper lipid included in pharmaceutical compositions provided herein is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), DOTAP, DOTMA, and phosphatidylcholine (PC) or a combination of any of the foregoing. In some aspects, the helper lipid is distearoylphosphatidylcholine (DSPC). In some aspects, the PEG-lipid conjugate included in pharmaceutical compositions provided herein is PEG-DMG. In some aspects, the PEG-DMG is PEG2000-DMG. In some aspects, the lipid nanoparticles comprise between about 20 mol % and 40 mol % of the cationic lipid; between about 25 mol % and 35 mol % of helper lipid; between about 25 mol % and 42 mol % cholesterol; and between about 0.5 mol % and 3 mol % PEG2000-DMG. In some aspects, the lipid nanoparticles comprise between about 20 mol % and 30 mol % of the cationic lipid; between about 30 mol % and 40 mol % of helper lipid; between about 34 mol % and 42 mol % cholesterol; and between about 1 mol % and 2 mol % PEG2000-DMG. In some aspects, the lipid nanoparticles comprise between about 22 mol % and 28 mol % of the cationic lipid; between about 31 mol % and 39 mol % of helper lipid; between about 35 mol % and 40 mol % cholesterol; and between about 1.25 mol % and 1.75 mol % PEG2000-DMG. In some aspects, the pharmaceutical composition has a total lipid:compound weight ratio of between about 8:1 and 40:1. In some aspects, pharmaceutical compositions provided herein have a total lipid:compound weight ratio of between about 10:1 and 30:1. In some aspects, pharmaceutical compositions provided herein have a total lipid:compound weight ratio of between about 15:1 and 30:1. In some aspects, pharmaceutical compositions provided herein have a total lipid:compound weight ratio of between about 10:1 and 25:1. In some aspects, pharmaceutical compositions provided herein comprise between about 20 w/w % and 60 w/w % of the cationic lipid.

Provided herein, in some embodiments, are methods of treating or preventing a metabolic disorder or condition in a subject that include administering to the subject an amount of a compound or pharmaceutical composition provided herein effective for treating or preventing the metabolic disorder or condition. In one aspect, the metabolic disorder or condition is obesity, liver steatosis, obesity-associated liver steatosis, liver inflammation, liver fibrosis, cardiovascular disease, atherosclerosis, coronary artery disease, diabetes, prediabetes, obesity-associated fat accretion, or obesity-related adipose tissue dysfunction. In one aspect, the administering decreases liver lipid accumulation, decreases liver triglyceride levels, decreases lipogenic and oxidative stress gene expression, decreases insulin resistance, reduces glycemia, or any combination thereof, as compared with administering a control compound or vehicle. In another aspect, the administering increases food consumption, reduces fat mass gain, increases circulating adiponectin levels, increases expression of adipogenic and thermogenic genes in adipose tissue, or any combination thereof, as compared with administering a control compound or vehicle. In yet another aspect, the administering increases food consumption, reduces fat mass gain, increases expression of adipogenic and thermogenic genes in adipose tissue, or any combination thereof, as compared with administering a control compound or vehicle. In one aspect, increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In some aspects, the adipose tissue is white adipose tissue. In some aspects, the compound or pharmaceutical composition is administered intravenously, orally, sublingually, intramuscularly, subcutaneously, intradermally, transdermally, intranasally, intraperitoneally, topically, or by a pulmonary route. In further aspects, the subject suffers from the metabolic disorder or condition, the subject is a healthy subject, the subject is an obese subject, or the subject is a non-obese subject. In one aspect, the subject consumes a high fat diet.

Provided herein, in some embodiments, are methods of treating or preventing an LBP-mediated disorder or condition in a subject comprising: administering to the subject an amount of a compound or pharmaceutical composition provided herein effective for treating or preventing an LBP-mediated disorder. In some aspects, the LBP-mediated disorder or condition is obesity, liver steatosis, obesity-associated liver steatosis, liver inflammation, liver fibrosis, cardiovascular disease, atherosclerosis, coronary artery disease, diabetes, prediabetes, obesity-associated fat accretion, or obesity-related adipose tissue dysfunction. In other aspects, the administering decreases liver lipid accumulation, decreases liver triglyceride levels, decreases lipogenic and oxidative stress gene expression, decreases insulin resistance, reduces glycemia, or any combination thereof, as compared with administering a control compound or vehicle. In one aspect, the administering increases food consumption, reduces fat mass gain, increases circulating adiponectin levels, increases expression of adipogenic and thermogenic genes in adipose tissue, or any combination thereof, as compared with administering a control compound or vehicle. In another aspect, the administering increases food consumption, reduces fat mass gain, increases expression of adipogenic and thermogenic genes in adipose tissue, or any combination thereof, as compared with administering a control compound or vehicle. In one aspect, increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In some aspects, the adipose tissue is white adipose tissue. In some aspects, the compound or pharmaceutical composition is administered intravenously, orally, sublingually, intramuscularly, subcutaneously, intradermally, transdermally, intranasally, intraperitoneally, topically, or by a pulmonary route. In some aspects, the subject suffers from the metabolic disorder or condition, the subject is a healthy subject, the subject is an obese subject, or the subject is a non-obese subject. In one aspect, the subject consumes a high fat diet.

Provided herein, in some embodiments, are methods of inhibiting expression of LBP in a subject comprising: administering to the subject an amount of a compound or pharmaceutical composition provided herein effective for inhibiting expression of LBP, thereby treating a metabolic disorder or condition in the subject. In one aspect, liver LBP expression, plasma LBP expression, or both liver and plasma LBP expression is inhibited as compared with administering a control compound or vehicle. In some aspects, the metabolic disorder or condition is obesity, liver steatosis, obesity-associated liver steatosis, liver inflammation, liver fibrosis, cardiovascular disease, atherosclerosis, coronary artery disease, diabetes, prediabetes, obesity-associated fat accretion, or obesity-related adipose tissue dysfunction. In one aspect, the administering decreases liver lipid accumulation, decreases liver triglyceride levels, decreases lipogenic and oxidative stress gene expression, decreases insulin resistance, reduces glycemia, or any combination thereof, as compared with administering a control compound or vehicle. In another aspect, the administering increases food consumption, reduces fat mass gain, increases circulating adiponectin levels, increases expression of adipogenic and thermogenic genes in adipose tissue, or any combination thereof, as compared with administering a control compound or vehicle. In yet another aspect, the administering increases food consumption, reduces fat mass gain, increases expression of adipogenic and thermogenic genes in adipose tissue, or any combination thereof, as compared with administering a control compound or vehicle. In one aspect, the increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In some aspects, the adipose tissue is white adipose tissue. In some aspects, the compound or pharmaceutical composition is administered intravenously, orally, sublingually, intramuscularly, subcutaneously, intradermally, transdermally, intranasally, intraperitoneally, topically, or by a pulmonary route. In some aspects, the subject suffers from the metabolic disorder or condition, the subject is a healthy subject, the subject is an obese subject, the subject or is a non-obese subject. In one aspect, the subject consumes a high fat diet.

### BRIEF DESCRIPTION OF THE DRAWINGS

FIGS. 1A-1J show lipogenesis-related gene expression in humans. FIG. 1A-1D) Plasma LBP concentration (1A), liver (1B), SAT (1C) and VAT (1D) LBP mRNA levels according to NAFLD. FIG. 1E) Bivariate correlations between liver LBP mRNA levels and plasma LBP concentration in morbidly obese participants. FIGS. 1F-1G) Bivariate correlations between liver LBP and SREBF1 (1F), FASN (1G) and ACACA (1H) mRNA levels. FIGS. 1I-1J) Effects of 6 months—LNP-Lbp UNA-siRNA administration in HFHS-fed mice on liver, iWAT and pgWAT Lbp mRNA levels (1I) and plasma Lbp concentration (1J) in male and female mice. *p<0.05, **p<0.01 and ***p<0.001 compared to CD; ^{†}p<0.05, ^{††}<0.01 and ^{†††}p<0.001 compared to HFHS.

FIGS. 2A-2J show the effect of 6 month-LNP-Lbp UNA-siRNA administration. FIGS. 2A-2H) Effects of 6 months-LNP-Lbp UNA-siRNA administration in HFHS-fed mice on representative standard Masson's trichrome staining histological liver slides (20×), lipid droplet (LD) count and area (2A, 2B), liver triglycerides (2C, 2D), histopathology scores (2E, 2F) and serum ALT activity (2G, 2H) in male (2A, 2C, 2E, 2G) and female (2B, 2D, 2F, 2H) mice. FIGS. 2I-2J) Effects of 6 months-LNP-Lbp UNA-siRNA administration in HFHS-fed mice on lipid metabolism (Fasn, Acaca, Srebf1, Scd1)-, oxidative stress (Gsta3, Gpx4, Sod2)-, glycolysis (Pdk1)-, fibrosis (Col3a1, Col4a1, Tgfb1)-, and inflammatory (Itgax, Ccl2, Tnf, Il6, Il10)-related gene expression in male (2I) and female (2J) mice. *p<0.05, **p<0.01 and ***p<0.001 compared to CD; ^{†}p<0.05, ^{††}<0.01 and ^{†††}p<0.001 compared to HFHS.

FIGS. 3A-3I show the effect of 8-week-LNP-Lbp UNA-siRNA administration. FIGS. 3A-F) Effects of 8 weeks-LNP-Lbp UNA-siRNA administration on Lbp mRNA levels and representative standard Masson's trichrome staining histological liver slides (20×), lipid droplet (LD) count and area (3A, 3B), histopathology scores (3C, 3D) and serum ALT activity (3E, 3F) in male (3A, 3C, 3E) and female (3B, 3D, 3F) diet-induced obese mice. FIGS. 3G-3H) Effects of 8 weeks-LNP-Lbp UNA-siRNA administration on lipid metabolism (Fasn, Acaca, Srebf1, Scd1)-, oxidative stress (Gsta3, Gpx4, Sod2)-, glycolysis (Pdk1)-, fibrosis (Col3a1, Col4a1, Tgfb1)-, and inflammatory (Itgax, Ccl2, Tnf, Il6, Il10)-related gene expression in male (3G) and female (3H) diet-induced obese mice. FIG. 3I) Effects of 8 week-LNP-Lbp UNA-siRNA administration on liver AMPK activity measured by the ratio ^{pThr172}AMPK/AMPK in male obese mice. *p<0.05, **p<0.01 and ***p<0.001 compared to obese-vehicle.

FIGS. 4A-4H show the effect of LNP-LBP siRNA administration on glucose metabolism and insulin levels. FIG. 4A-4F) Effects of 6 months-LNP-Lbp UNA-siRNA administration on insulin at at 4, 8 and 25 weeks (4A), insulin change (4B), blood glucose during ITT (4C), glycemia (AUC) in ITT (4D) and fasting glucose (4E) in HFHS-fed female and male mice. *p<0.05, **p<0.01 and ***p<0.001 compared to CD; ^{†}p<0.05 and ^{††}<0.01 compared to HFHS. FIG. 4F-4H) Effect of 8 weeks-LNP-Lbp UNA-siRNA administration on blood glucose during ITT (4F), glycemia (AUC) in ITT (4G) and fasting glucose (4H) in female and male obese mice. *p<0.05 and **p<0.01 compared to obese-vehicle; ^{‡}p<0.05, ^{‡‡}p<0.01 and ^{‡‡‡}p<0.001 compared to female mice.

FIGS. 5A-5I show the effect of Lbp gene knockdown in Hepa1-6 cells. FIG. 5A-5G) Effects of Lbp gene knockdown in Hepa1-6 cell line on Lbp gene expression (5A), mitochondrial respiration parameters (including basal respiration, ATP production, proton leak and spare respiratory capacity) (5B), expression of inflammatory (Tnf, Crp)-, fibrosis (Col4a1, Tgfb1)- and metabolic (Srebf1, Scd1, Pdk1)-related genes (5C-5F), AMPK activity (measured by the ratio ^{pThr172}AMPK/AMPK) (5G) and gene expression markers of oxidative stress (Gpx4, Sod2) (5H) and endoplasmic reticulum (ER) stress (Syvn1, Atf6 and Hspa5) (5I) in vehicle (BSA5%, 24 h)- and palmitate (200 μM, 24 h)-treated Hepa1-6 cells. *p<0.05, **p<0.01 and ***p<0.001 compared to shC; ^{†}p<0.05, ^{††}<0.01 and ^{†††}p<0.001 compared to BSA.

FIGS. 6A-6D show a schematic of exemplary experimental designs of metabolic experiments. Metabolic experiments consisted of the following treatments: Non-treated CD-fed mice for 25 weeks (6A), non-treated HFHS-fed mice for 25 weeks (6B), weekly LNP-Lbp UNA-siRNA (3 mg/kg) treated HFHS-fed mice for 25 weeks (6C), and weekly LNP-Lbp UNA-siRNA (3 mg/kg) treated HFHS-fed mice only for the last 8 weeks (6D). CD: Control diet; HFHS: High fat and high sucrose diet; V: Weekly vehicle administration; and L: Weekly LNP-Lbp UNA-siRNA (3 mg/kg) administration.

FIGS. 7A-7C show the effect of chemically modified (UNA-si) and unmodified (si) siRNAs targeting Lbp mRNA. FIG. 7A) Effect of several doses of chemically unmodified and modified siRNA (si107, si69108, si109) against the Lbp mRNA sequence in Hepa1-6 cells. FIG. 7B) Effect of chemically unmodified (si6910, 1 and 3 mg/kg) and chemically modified (UNA-si69108), 1 and 3 mg/kg) LNP-Lbp UNA-siRNA on plasma LBP at day 3, 6, 10 and 12 after injection. FIG. 7C) Effect of chemically modified (UNA-si69108, 1 and 3 mg/kg) LNP-Lbp UNA-siRNA on liver Lbp gene expression 3 days after second injection. *p<0.05, **p<0.01 and ***p<0.001 compared to 0 pmol/L or vehicle.

FIGS. 8A-8C show the effect of LNP-Lbp UNA-siRNA administration on body weight, liver metabolism, and cytokine and chemokine gene expression. FIG. 8A) Effect of 6 month-LNP-Lbp UNA-siRNA administration on monthly body weight. FIG. 8B) Effect of 8 week-LNP-Lbp UNA-siRNA administration on weekly body weight. FIG. 8C) Effect of 6 month-LNP-Lbp UNA-siRNA administration on IL-10, IL-6-, IFNγ-, IL12p70-, TNFα- and MCP1-levels. *p<0.05 and **p<0.01 compared to CD; ^{†}p<0.05 and ^{††}<0.01 compared to HFHS; ^{‡‡‡}p<0.001 compared to female mice.

FIGS. 9A-9G show the effect of siRNA administration targeting Lbp mRNA on food intake, adiposity, and circulating adiponectin levels. FIG. 9A-9E) Effects of 6 months-LNP-UNA-siRNA LBP administration on plasma LBP levels at 4, 8 and 25 weeks (9A), food consumption mean (9B), body weight (9C), fat mass (9D) and lean mass gain (9E). FIGS. 9F-9G) Effects of 6 months-LNP-UNA-siRNA LBP administration on circulating leptin (9F) and adiponectin (9G) at 25 weeks. *p<0.05, **p<0.01 and ***p<0.001 compared to CD; ^{†}p<0.05, ^{††}<0.01 and ^{†††}p<0.001 compared to HFHS; ^{‡}p<0.05, ^{‡‡}p<0.01 and ^{‡‡‡}p<0.001 compared to female mice.

FIGS. 10A-10E show the effect of 6 month-administration of siRNA targeting Lbp mRNA on pgWAT gene expression related to adipogenesis and thermoregulation. FIGS. 10A-10E) Effects of 6 months-LNP-UNA-siRNA LBP administration on pgWAT Adipoq, Slc2a4, Fabp4, Scd1, Fasn, Plin1, Pparg, Lbp, Lep, Il6, Lipe, Mgll, Ascl1, Ucp1, Nrg4 and Adbr3 gene expression (10A-10D) and on UCP1 protein levels (10E). *p<0.05, **p<0.01 and ***p<0.001 compared to CD; ^{†}p<0.05 and ^{††}<0.01 compared to HFHS; ^{‡}<0.05, ^{‡‡}p<0.01 and ^{‡‡‡}p<0.001 compared to female mice.

FIGS. 11A-11G show the effect of 8 week-administration of siRNA targeting Lbp mRNA on pgWAT gene expression related to adipogenesis and thermoregulation in obese mice. FIGS. 11A-11F) Effects of 8 weeks-LNP-UNA-siRNA LBP administration on plasma Lbp (11A), on pgWAT Adipoq, Slc2a4, Fabp4, Scd1, Fasn, Plin1, Pparg, Lbp, Lep, Il6, Lipe, Mgll, Ucp1, Nrg4 and Adbr3 gene expression (11B-11F) and on UCP1 protein levels (11G) in diet-induced obese mice. *p<0.05, **p<0.01 and ***p<0.001 compared to obese-vehicle; ^{‡}p<0.05, ^{‡‡}p<0.01 and ^{‡‡‡}p<0.001 compared to female mice.

FIGS. 12A-12I show the short-term effect of siRNA administration targeting Lbp mRNA on abundance of fat tissue and gene expression in females with different body weight. FIG. 12A-121) Effects of 12 weeks-LNP-UNA-siRNA LBP administration on liver Lbp mRNA (12A), body weight (12B), percentage of tissue (iWAT, pgWAT and liver) weight normalized by body weight (12C), expression of adipogenic (Adipoq, Fabp4, Slc2a4, Scd1, Plin1, Pparg)-, lipolitic and thermogenic (Adbr3, Lipe, Mgll, Ucp1)- and obesity (Il6, Lbp, Lep)-related genes in pgWAT (12D-12F) and iWAT (12G-12I) in 8 week-old female mice fed with HFD (60%). *p<0.05, **p<0.01 and ***p<0.001 compared to vehicle; ^{†}p<0.05 and ^{††}<0.01 compared to LBW. LBW: Low body weight mice; HBW: High body weight mice.

FIGS. 13A-13K show the effects of white adipose tissue-specific Lbp gene knockdown. FIG. 13A) Representative diagram of specific iWAT and pgWAT Lbp gene knockdown experimental design. FIGS. 13B-13L) Effects of specific iWAT and pgWAT Lbp gene knockdown on weekly body weight (13B), body weight gain (13C), iWAT Lbp (13D), adipogenic genes (Fasn, Ascl1, Adipoq, Fabp4, Plin1 and Slc2a4) (13E), iWAT thermogenic genes (Adrb3, Nrg4, Ucp1) (13F), pgWAT Lbp (13G), adipogenic genes (Fasn, Ascl1, Adipoq, Fabp4, Plin1 and Slc2a4) (13H), thermogenic genes (Adrb3, Nrg4, Ucp1) (13I) in HFHS-fed female and male mice. Effects of specific iWAT and pgWAT Lbp gene knockdown on iWAT (13J) and pgWAT (13K) inflammatory (Il6, Ccl2, Tnf, Cd14, Itgax) gene expression in HFHS-fed female and male mice. *p<0.05, **p<0.01 and ***p<0.001 compared to shC; ^{†}p<0.05 and ^{††}<0.01 compared to female mice.

FIGS. 14A-14D show the effect of 6 month-administration of siRNA targeting Lbp mRNA on body weight, body fat and food consumption. FIGS. 14A-14D) Effect of 6 months-LNP-UNA-siRNA LBP administration on weekly body weight (14A), fat (14B) and lean (14C) mass and food consumption (14D). *p<0.05, *p<0.01 and *p<0.001 compared to CD; ^{†}p<0.05, ^{††}<0.01 and ^{†††}p<0.001 compared to HFHS; ^{‡}p<0.05, ^{‡‡}p<0.01 and ^{‡‡‡}p<0.001 compared to female mice.

FIGS. 15A-15C show the effect the effect of 8 week-administration of siRNA targeting Lbp mRNA on weight gain, body fat, and food consumption. FIGS. 15A-15C) Effect of 8 weeks-LNP-UNA-si69108 administration on weekly food consumption (15A) and weekly body weight (15B). FIG. 15C) Body weight gain (from week 1 to 16 and from week 17 to 25), body weight at week 17 and 25, and fat and lean mass at week 24. ^{‡‡}p<0.01 and ^{‡‡‡}p<0.001 compared to female mice.

FIGS. 16A-16F show the effect of 6 month/8 week-administration of siRNA targeting Lbp mRNA on iWAT gene expression related to adipogenesis and thermoregulation. FIGS. 16A-16C) Effects of 6 months-LNP-UNA-siRNA LBP administration on iWAT Adipoq, Slc2a4, Fabp4, Scd1, Fasn, Plin1, Pparg, Lbp, Lep, Il6, Ucp1 and Adbr3 gene expression. *p<0.05, **p<0.01 and ***p<0.001 compared to CD; ^{†}p<0.05 compared to HFHS. FIGS. 16D-16F) Effects of 8 weeks-LNP-UNA-siRNA LBP administration on iWAT Adipoq, Slc2a4, Fabp4, Scd1, Fasn, Plin1, Pparg, Lbp, Lep, Il6, Ucp1 and Adbr3 gene expression in diet-induced obese mice. *p<0.05 and ***p<0.001 compared to obese-vehicle; ^{‡}p<0.05, ^{‡‡}p<0.01 and ^{‡‡‡}p<0.001 compared to female mice.

### DETAILED DESCRIPTION

The present disclosure provides for compositions and methods for the treatment of metabolic disorders and conditions. In particular, compositions and methods of the disclosure provide for decreasing expression of lipopolysaccharide binding protein (LBP) in the liver and/or plasma for treating metabolic disorders and conditions related to obesity, including liver steatosis and adipose tissue dysfunction, for example.

### Compounds for RNA Interference

Provided herein, in some embodiments, are compounds for RNA interference (RNAi). RNAi is a gene silencing process in which RNAs inhibit or reduce gene expression by targeting mRNA molecules. Generally, gene silencing by RNAi inhibits or reduces gene expression at the posttranscriptional level. For example, RNAi can result in mRNA degradation or in repression of translation. Compounds provided herein can target expression of LBP, resulting in knockdown of LBP expression.

Compounds provided herein generally include a sense strand (also referred to as passenger strand) and an antisense strand (also referred to a guide strand). The sense and antisense strands can be substantially complementary to each other. As used herein, the term “antisense strand” refers to a polynucleotide or region of a polynucleotide that is at least substantially (e.g., about 80% or more) complementary to a target nucleic acid of interest. The antisense strand can be about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and any number or range in between, complementary to a target nucleic acid of interest. Similarly, an antisense strand of a double-stranded RNA compound, such as a compound provided herein, can be at least substantially complementary to its sense strand. In some embodiments, the antisense strand of compounds provided herein is substantially complementary to Lbp mRNA or a region thereof.

As used herein the terms “complementary” and “complementarity” are meant to refer to the ability of polynucleotides to form base pairs with one another. Base pairs are typically formed by hydrogen bonds between nucleotides in anti-parallel polynucleotide strands. Complementary polynucleotide strands can base pair in the Watson-Crick manner (e.g., A to T, A to U, C to G), or in any other manner that allows for the formation of duplexes. As persons skilled in the art appreciate, when using RNA as opposed to DNA, uracil rather than thymine is the base that is considered to be complementary to adenosine.

Perfect complementarity or 100% complementarity refers to a situation in which each nucleotide of one polynucleotide strand can hydrogen bond with a nucleotide of an antiparallel polynucleotide strand. Less than perfect complementarity refers to a situation in which some, but not all, nucleotides of two strands can hydrogen bond with each other. For example, for two 20mers, if only two base pairs on each strand can hydrogen bond with each other, the polynucleotide strands exhibit complementarity. As another example, if 18 nucleotides out of 20 nucleotides on each strand can hydrogen bond with each other, the polynucleotide strands exhibit 90% complementarity. “Substantial complementarity” refers to polynucleotide strands exhibiting 80% or greater complementarity, excluding regions of the polynucleotide strands, such as overhangs, that are selected to be non-complementary. Accordingly, complementarity does not consider overhangs that are selected to not be similar or complementary to the nucleotides on the antiparallel strand, unless context clearly indicates otherwise.

An antisense strand and a sense strand can include mismatches. For example, an antisense strand and a sense strand can include nucleotide sequences that are not complementary to each other. A sense strand and an antisense strand can have 1 mismatch, 2 mismatches, 3 mismatches, 4 mismatches, 5 mismatches, or more mismatches. Mismatches can be contiguous or can be located anywhere along the sense and antisense strands. As another example, an antisense strand can have perfect complementarity to a sense strand and include mismatches with respect to the target mRNA. An antisense strand can have 1 mismatch, 2 mismatches, 3 mismatches, 4 mismatches, 5 mismatches, or more mismatches with respect to the target mRNA. Mismatches between a sense strand and an antisense strand or an antisense strand and a target mRNA can result in a bulge. One or more mismatches between an antisense strand and a target mRNA can result in translational repression rather than degradation of the target mRNA. In some embodiments, the target mRNA of compounds provided herein is Lbp mRNA.

The sense and antisense strands of compounds provided herein can each independently include 19-31 nucleotides or monomers. As used herein, the term “monomer” includes unmodified nucleotides, modified nucleotides, and UNA monomers, unless specified otherwise and/or unless context clearly indicates otherwise. In one aspect, the sense and antisense strands of compounds provided herein each independently include 19 monomers, 20 monomers, 21 monomers, 22 monomers, 23 monomers, 24 monomers, 25 monomers, 26 monomers, 27 monomers, 28 monomers, 29 monomers, 30 monomers, or 31 monomers. In another aspect, the sense and antisense strands of compounds provided herein include 19 monomers.

In some embodiments, provided herein are compounds for RNA interference comprising (A)(i) a sense strand comprising a sequence having at least 80% identity to a sequence of 5′-GUUUAAGGGUGAAAUUUUU-3′ (SEQ ID NO:13), and (ii) an antisense strand comprising a sequence having at least 80% identity to a sequence of 5′-AAAAAUUUCACCCUUAAAC-3′ (SEQ ID NO:14); (B)(i) a sense strand comprising a sequence having at least 80% identity to a sequence of 5′-GCCUACCUGAGGACAGUAA-3′ (SEQ ID NO:15), and (ii) an antisense strand comprising a sequence having at least 80% identity to a sequence of 5′-UUACUGUCCUCAGGUAGGC-3′ (SEQ ID NO:16); or (C)(i) a sense strand comprising a sequence having at least 80% identity to a sequence of 5′-GAUGUGCACAUAUCAGGAA-3′ (SEQ ID NO:17), and (ii) an antisense strand comprising a sequence having at least 80% identity to a sequence of 5′-UUCCUGAUAUGUGCACAUC-3′ (SEQ ID NO:18); wherein the compound comprises at least one UNA monomer and at least one chemically modified monomer.

As used herein, the term “UNA monomer” or “unlocked nucleic acid monomer” refers to a small organic molecule based on a propane-1,2,3-tri-yl-trisoxy structure as shown below:

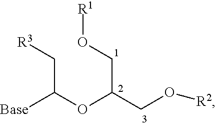

wherein R¹ and R² can be H or R¹ and R² can be phosphodiester linkages (the O to which R¹ or R² is attached would be part of the phosphodiester linkage), Base can be a natural or modified nucleobase, and R³ is a functional group selected from OR⁴, SR⁴, NR⁴R⁴, —NH(C═O)R⁴, morpholino, morpholin-1-yl, piperazin-1-yl, or 4-alkanoyl-piperazin-1-yl, where R⁴ is the same or different for each occurrence, and can be H, alkyl, a cholesterol, a lipid molecule, a polyamine, an amino acid, or a polypeptide. Examples of a nucleobase include uracil, thymine, cytosine, 5-methylcytosine, adenine, guanine, and inosine. Further examples include natural and non-natural nucleobase analogues and UNA monomers found in U.S. 2018/0362985, for example. The terms “UNA monomer,” “unlocked nucleic acid monomer,” and “unlocked nucleomonomer agent” can be used interchangeably, unless context clearly indicates otherwise.

In general, because the UNA monomers are not nucleotides, they can exhibit at least four forms in an oligomer. For example, an UNA monomer can be an internal monomer in an oligomer, where the UNA monomer is flanked by other monomers on both sides. In this form, the UNA monomer can participate in base pairing when the oligomer is a duplex, for example, and there are other monomers with nucleobases in the duplex.

As another example, an UNA monomer can be a monomer in an overhang of an oligomer duplex, where the UNA monomer is flanked by other monomers on both sides. In this form, the UNA monomer does not participate in base pairing. Because the UNA monomers are flexible organic structures, unlike nucleotides, the overhang containing an UNA monomer can be a flexible terminator for the oligomer.

As yet another example, an UNA monomer can be a terminal monomer in an overhang of an oligomer, where the UNA monomer is attached to only one monomer at either the propane-1-yl position or the propane-3-yl position. In this form, the UNA monomer does not participate in base pairing.

As a further example, because the UNA monomers are flexible organic structures, unlike nucleotides, the overhang containing a UNA monomer can be a flexible terminator for the oligomer and assume different conformations. Thus, UNA oligomers having a terminal UNA monomer are significantly different in structure from conventional nucleic acid agents, such as siRNAs that do not include UNA monomers. For example, siRNAs that do not include UNA monomers may require that terminal monomers or overhangs in a duplex be stabilized. In contrast, the conformability of a terminal UNA monomer can provide UNA oligomers with different properties.

Oligomeric compounds comprising one or more UNA monomers (UNA oligomers, such as modified siRNAs, for example) can be prepared by automated oligonucleotide synthesis as known to a person skilled in the art. The incorporation of the UNA monomers into the oligonucleotides such as compounds provided herein can follow standard methods for oligonucleotide synthesis, work-up, purification and isolation (F. Eckstein, Oligonucleotides and Analogues, IRL Press, Oxford University Press, 1991) with modifications as published (Johannsen, M. W. et al., Org. Biomol. Chem., 2011, 9, 243).

As used herein, “UNA-N” refers to an UNA monomer, where “N” is any base. Exemplary bases include adenine (A), guanine (G), cytosine (C), uracil (U; generally present in RNA), and thymine (T; generally present in DNA). Accordingly, UNA monomers present in RNA include UNA-A, UNA-U, UNA-C, UNA-G, or any combination thereof. As used herein, “UNA-N” generally refers to an UNA monomer in an RNA molecule, unless context clearly indicates otherwise.

One or more UNA monomers can be included at any position of the sense strand, the antisense strand, or both the sense and antisense strands of compounds provided herein. In one aspect, G at a first position from the 5′ end of the sense strand of compounds provided herein is replaced by an UNA monomer. Accordingly, instead of G at the 5′ end of the sense strand of compounds provided herein (e.g., SEQ ID NOs:13, 15, or 17), an UNA monomer is present. In another aspect, the UNA monomer at the first position from the 5′ end of the sense strand is UNA-G, UNA-U, UNA-A, or UNA-C. In yet another aspect, the UNA monomer at the first position from the 5′ end of the sense strand is UNA-G. UNA monomers at the first position from the 5′ end of the sense strand can be linked to a 5′ unmodified monomer or a 5′ chemically modified monomer by any linkage. In one aspect, the UNA monomer at the first position from the 5′ end of the sense strand is connected to a 5′ unmodified monomer or a 5′ chemically modified monomer by a phosphorothioate linkage, a chiral phosphorothioate linkage, or a phosphorodithioate linkage.

In another aspect, the sense strand, the antisense strand, or both the sense strand and the antisense strand of compounds provided herein include an UNA monomer added at the 3′end. The UNA monomer added at the 3′ end of the sense strand, the antisense strand, or both the sense strand and the antisense strand of compounds provided herein can be UNA-U, UNA-G, UNA-A, UNA-C, or any combination thereof. In an aspect, the UNA monomer added at the 3′ end is UNA-U. UNA monomers can be added to the 3′ end by any linkage. In one aspect, the UNA monomer added at the 3′ end is connected to a 3′ unmodified monomer or a 3′ chemically modified monomer by a phosphorothioate linkage, a chiral phosphorothioate linkage, or a phosphorodithioate linkage. As used herein, the symbol “/*/” refers to the presence of a phosphorothioate (PS) bond, unless context clearly indicates otherwise.

Compounds provided herein can include at least one modified or chemically modified monomer. Modified or chemically modified monomers can include modified or chemically modified ribonucleotides or deoxyribonucleotides. As used herein, “rN” refers to ribonucleotides, “mN” refers to 2′-O-methyl modified ribonucleotides, “dN” refers to deoxyribonucleotides, where N can be any base, such as adenine (A), guanine (G), cytosine (C), uracil (U; generally present in RNA), and thymine (T; generally present in DNA), for example. Alternatively, upper case letters may be used herein to designate ribonucleotides, and lower case letters may be used to designate deoxyribonucleotides.

In one aspect, the sense strand, the antisense strand, or both the sense strand and the antisense strand of compounds provided herein include at least one chemically modified monomer. In another aspect, the sense strand, the antisense strand, or both the sense strand and the antisense strand include 1 to 19 chemically modified monomers. In yet another aspect, the sense strand and the antisense strand each independently include 1 chemically modified monomer. 2 chemically modified monomers, 3 chemically modified monomers, 4 chemically modified monomers, 5 chemically modified monomers, 6 chemically modified monomers, 7 chemically modified monomers, 8 chemically modified monomers, 9 chemically modified monomers, 10 chemically modified monomers, 11 chemically modified monomers, 12 chemically modified monomers, 13 chemically modified monomers, 14 chemically modified monomers, 15 chemically modified monomers, 16 chemically modified monomers, 17 chemically modified monomers, 18 chemically modified monomers, 19 chemically modified monomers, or more chemically modified monomers.

Any non-natural, modified, or chemically modified monomers can be included in compounds provided herein. Exemplary non-natural, modified, and chemically modified monomers include 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, inverted deoxyabasic monomer residues, 3′-end stabilized nucleotides, 3′-glyceryl nucleotides, 3′-inverted abasic nucleotides, 3′-inverted thymidine, L-thyrmidine, locked nucleic acid nucleotides, 2′-0,4′-C-methylene-(D-ribofuranosyl) nucleotides, 2′-methoxyethoxy (MOE) nucleotides, constrained methoxyethyl (cMOE) nucleotides or nucleoside analogs, constrained ethyl (cEt) nucleotides or nucleoside analogs, 2′-methyl-thio-ethyl nucleotides, 2′-deoxy-2′-fluoro nucleotides, 2′-O-methyl nucleotides, 2′-amino nucleotides, 2′-O-amino nucleotides, 2′-C-allyl nucleotides, 2′-O-allyl nucleotides, N6-methyladenosine nucleotides, nucleotide monomers with modified bases, such as 5-(3-amino)propyluridine, 5-(2-mercapto)ethyluridine, 5-bromouridine, 8-bromoguanosine, or 7-deazaadenosine, 2′-O-aminopropyl substituted nucleotides, 2′-O-guanidinopropyl substituted nucleotides, pseudouridines, 1-methylpseudouridine, 5-methylcytosine, 5-methoxyuridine, nucleotides prepared by replacing the 2′—OH group of a nucleotide with a 2′-R, a 2′-OR, a 2′-halogen, a 2′-SR, or a 2′-amino, 2′-azido, where R can be H, alkyl, fluorine-substituted alkyl, alkenyl, or alkynyl, nucleotides prepared by replacing the 2′-OH group of a nucleotide with a 2′-R or 2′-OR, where R can be CN, CF₃, alkylamino, or aralkyl, nucleotides with a modified sugar such as an F-HNA, an HNA, a CeNA, a bicyclic sugar, or an LNA, nucleotides such as 2′-oxa-3′ aza-4′a-carbanucleoside monomers, 3-hydroxymethyl-5-(1H-1,2,3-triazol)-isoxazolidine monomers, and 5′-triazolyl-2′-oxa-3′-aza-4′a-carbanucleoside monomers, and others.

In one aspect, chemically modified monomers included in compounds provided herein are methylated monomers. In another aspect, chemically modified monomers included in compounds provided herein include 2′-O-methyl modified monomers. In yet another aspect, chemically modified monomers included in compounds provided herein include 2′-O-methyl modified monomers, inverted thymidine monomers, L-thymidine monomers, glyceryl nucleotides, 2′-methoxyethoxy nucleotides, 2′-deoxy-2′-fluoro ribonucleotides, or any combination thereof.

Compounds provided herein can include one or more deoxy T monomer. In one aspect, a deoxy T monomer is connected to the UNA monomer added at the 3′end of the sense strand, the antisense strand, or both the sense strand and the antisense strand of compounds provided herein. In another aspect, a deoxy T monomer is connected to the UNA monomer added at the 3′end of the sense strand, the antisense strand, or both the sense strand and the antisense strand of compounds provided herein by a phosphorothioate linkage, a chiral phosphorothioate linkage, or a phosphorodithioate linkage. Accordingly, deoxyribonucleotides such as dT can be included in RNA compounds.

Provided herein, in some embodiments, are compounds for RNA interference comprising: (A)(i) a sense strand comprising a sequence having at least 80% identity to a sequence of SEQ ID NO:1, and (ii) an antisense strand comprising a sequence having at least 80% identity to a sequence of SEQ ID NO:2; (B)(i) a sense strand comprising a sequence having at least 80% identity to a sequence of SEQ ID NO:3, and (ii) an antisense strand comprising a sequence having at least 80% identity to a sequence of SEQ ID NO:4; or (C)(i) a sense strand comprising a sequence having at least 80% identity to a sequence of SEQ ID NO:5, and (ii) an antisense strand comprising a sequence having at least 80% identity to a sequence of SEQ ID NO:6. In one aspect, compounds provided herein further include a deoxy T monomer at the 3′ end of the sense strand, the antisense strand, or both the sense and antisense strands. The deoxy T monomer can be linked to the 3′ end by a phosphorothioate linkage, a chiral phosphorothioate linkage, or a phosphorodithioate linkage. In one aspect, a deoxy T monomer is linked to an UNA monomer added at the 3′end of the sense strand, the antisense strand, or both the sense strand and the antisense strand of compounds provided herein. In another aspect, a deoxy T monomer is linked to an UNA monomer added at the 3′end of the sense strand, the antisense strand, or both the sense strand and the antisense strand of compounds provided herein by a phosphorothioate linkage, a chiral phosphorothioate linkage, or a phosphorodithioate linkage.

Provided herein, in some embodiments, are compounds for RNA interference comprising: (A)(i) a sense strand comprising a sequence having at least 80% identity to a sequence of SEQ ID NO:7, and (ii) an antisense strand comprising a sequence having at least 80% identity to a sequence of SEQ ID NO:8; (B)(i) a sense strand comprising a sequence having at least 80% identity to a sequence of SEQ ID NO:9, and (ii) an antisense strand comprising a sequence having at least 80% identity to a sequence of SEQ ID NO:10; or (C)(i) a sense strand comprising a sequence having at least 80% identity to a sequence of SEQ ID NO:11, and (ii) an antisense strand comprising a sequence having at least 80% identity to a sequence of SEQ ID NO:12.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence. As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

In some aspects, sense and/or antisense strands of compounds provided herein have at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, and any number or range in between, identity to a reference sequence. In one aspect, sense and/or antisense strands of compounds provided herein have 100% identity to a reference sequence.

In some aspects, compounds provided herein selectively inhibit lipopolysaccharide binding protein (LBP) expression. In one aspect, LBP expression is selectively inhibited in the liver, in plasma, or in both the liver and in plasma. LBP expression can be reduced by about 0.1%, about 0.5%, about 1%, about 2.5%, about 5%, about 10%, about 20%, about 30%, about 40%, about 50%, about 60%, about 70%, about 80%, about 90%, about 95%, about 96%, about 97%, about 98%, about 99%, about 99.5%, about 100%, and any number or range in between, as compared to expression in the absence of a compound provided herein or as compared to the presence of a control compound. LBP expression can also be reduced about one-fold, about two-fold, about three-fold, about four-fold, about five-fold, about six-fold, about seven-fold, about eight-fold, about nine-fold, about ten-fold, about 20-fold, about 30-fold, about 40-fold, about 50-fold, about 60-fold, about 70-fold, about 80-fold, about 90-fold, about 100-fold, or more, and any number or range in between, as compared to expression in the absence of a compound provided herein or as compared to the presence of a control compound. In an aspect, there is no LBP expression. In another aspect, there is no detectable LBP expression.

### Pharmaceutical Compositions

Provided herein, in some embodiments, are pharmaceutical compositions comprising any of the compounds provided herein. Pharmaceutical compositions provided herein can further include a pharmaceutically acceptable carrier. For example, pharmaceutical compositions provided herein can include as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. Pharmaceutical compositions provided herein can include lipids as pharmaceutically acceptable carriers, such as lipids that form lipid nanoparticles, liposomes, micelles, and microspheres, for example. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., siRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane, release at the cytoplasm (for RNAs), and so on. Successful nucleic acid-based therapy thus depends upon the ability of the vector to deliver the nucleic acids to the target sites inside of the cells in order to obtain sufficient levels of a desired activity such as expression of a gene or interference of translation.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including siRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int. J. Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed hereinbelow.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation.

### Lipid-siRNA (UNA Oligomer) Formulations

An siRNA or UNA oligomer as disclosed herein or a pharmaceutically acceptable salt thereof can be incorporated into a lipid formulation (i.e., a lipid-based delivery vehicle).

In the context of the present disclosure, a lipid-based delivery vehicle typically serves to transport a desired UNA oligomer to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing a UNA oligomer of the present disclosure. In some embodiments, the lipid-based delivery vehicle comprises a nanoparticle or a bilayer of lipid molecules and a UNA oligomer of the present disclosure. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic UNA oligomer molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the UNA oligomer is fully encapsulated within the lipid portion of the lipid formulation such that the UNA oligomer in the lipid formulation is resistant in aqueous solution to nuclease degradation. In other embodiments, the lipid formulations described herein are substantially non-toxic to mammals such as humans.

The lipid formulations of the disclosure also typically have a total lipid:UNA oligomer ratio (mass/mass ratio) of from about 1:1 to about 100:1, from about 1:1 to about 50:1, from about 2:1 to about 45:1, from about 3:1 to about 40:1, from about 5:1 to about 38:1, or from about 10:1 to about 40:1, or from about 15:1 to about 35:1, or from about 20:1 to about 40:1; or from about 25:1 to about 35:1; or from about 27:1 to about 32:1; or from about 28:1 to about 32:1; or from about 29:1 to about 31:1. In some preferred embodiments, the total lipid:UNA oligomer ratio (mass/mass ratio) is from about 25:1 to about 35:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise a UNA oligomer, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol.

In the nucleic acid-lipid formulations, the UNA oligomer may be fully encapsulated within the lipid portion of the formulation, thereby protecting the nucleic acid from nuclease degradation. In preferred embodiments, a lipid formulation comprising a UNA oligomer is fully encapsulated within the lipid portion of the lipid formulation, thereby protecting the nucleic acid from nuclease degradation. In certain instances, the UNA oligomer in the lipid formulation is not substantially degraded after exposure of the particle to a nuclease at 37° C. for at least 20, 30, 45, or 60 minutes. In certain other instances, the UNA oligomer in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the UNA oligomer is complexed with the lipid portion of the formulation. One of the benefits of the formulations of the present disclosure is that the nucleic acid-lipid compositions are substantially non-toxic to mammals such as humans.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where/and I₀ refers to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of UNA oligomer-liposomes. In some embodiments, the nucleic acid-lipid composition comprises a plurality of UNA oligomer-cationic liposomes. In some embodiments, the nucleic acid-lipid composition comprises a plurality of UNA oligomer-lipid nanoparticles.

In some embodiments, the lipid formulations comprise UNA oligomer that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the UNA oligomer encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the UNA oligomers described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) a UNA oligomer of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In one some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. Exemplary cationic lipids (including ionizable cationic lipids), helper lipids (e.g., neutral lipids), sterols, and ligand-containing lipids (e.g., PEG-lipids) are described hereinbelow.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA). Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In some embodiments, the lipid formulation comprises the cationic lipid with Formula I as described in WO 2018/078053. In this context, the disclosure of WO 2018/078053 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula (V)

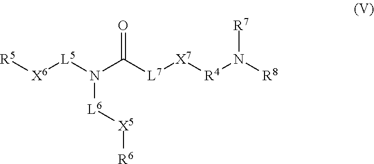

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of —CH((CH₂)ₚCH₃)((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is —C(O)O—, whereby —C(O)O—R⁶ is formed, X⁶ is —C(O)O— whereby —C(O)O—R⁵ is formed, L⁵ and L⁶ are each independently a linear C₃-C₇ alkyl, L⁷ is absent, R⁵ is —CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group consisting of

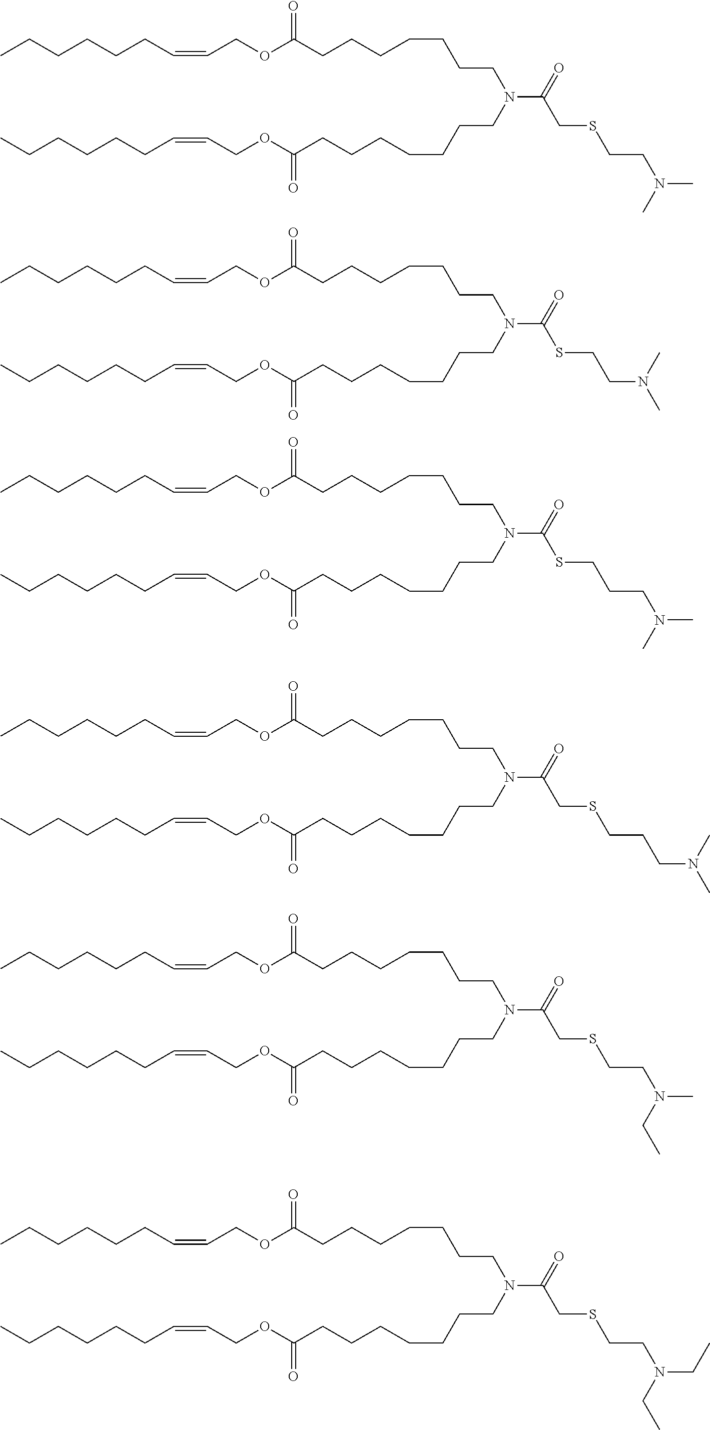

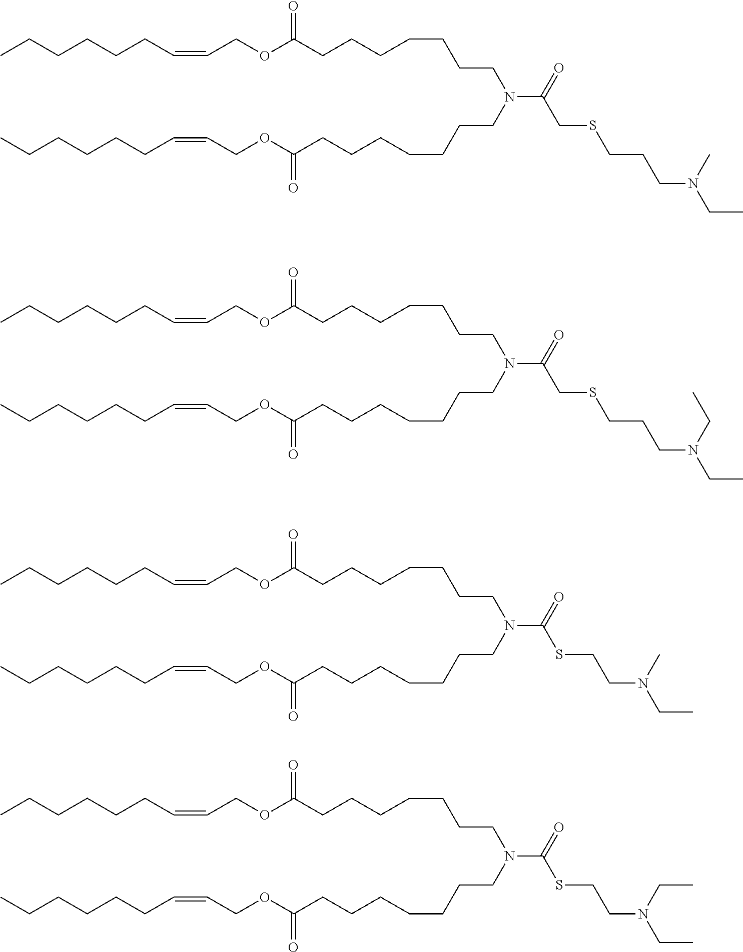

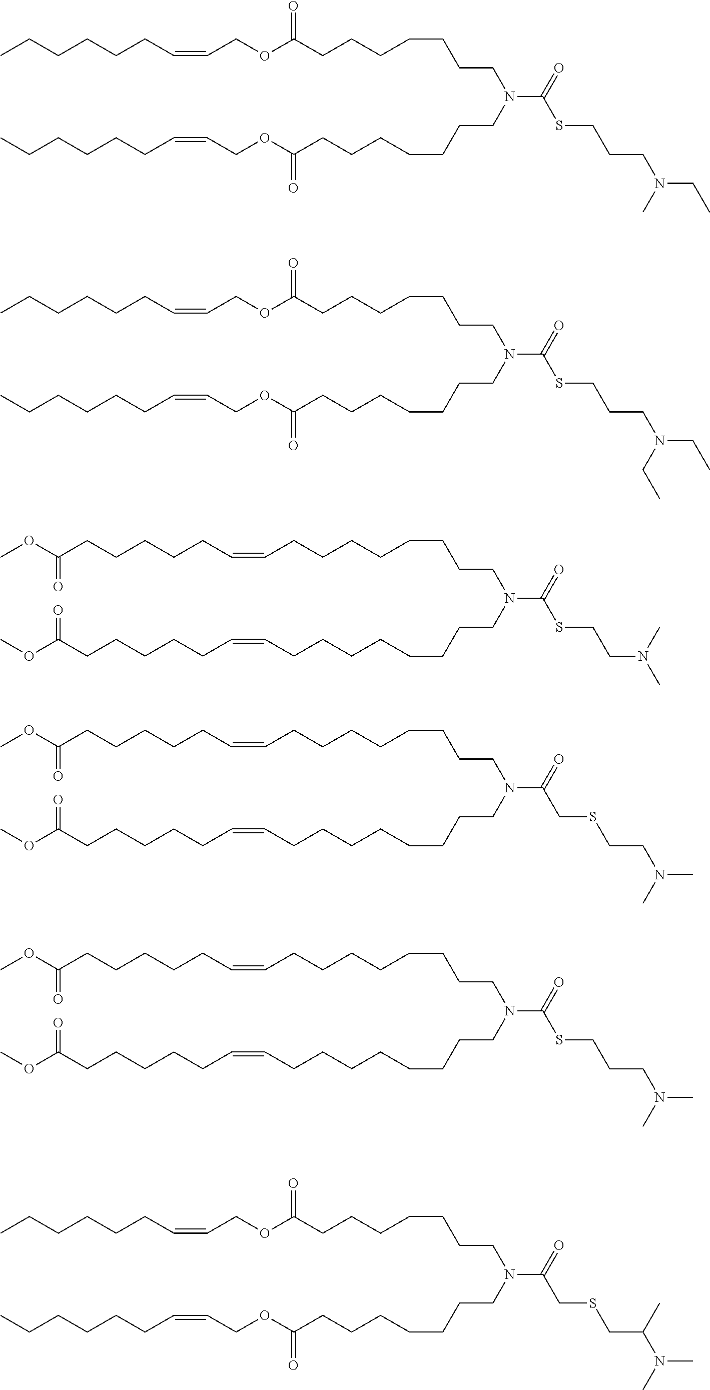

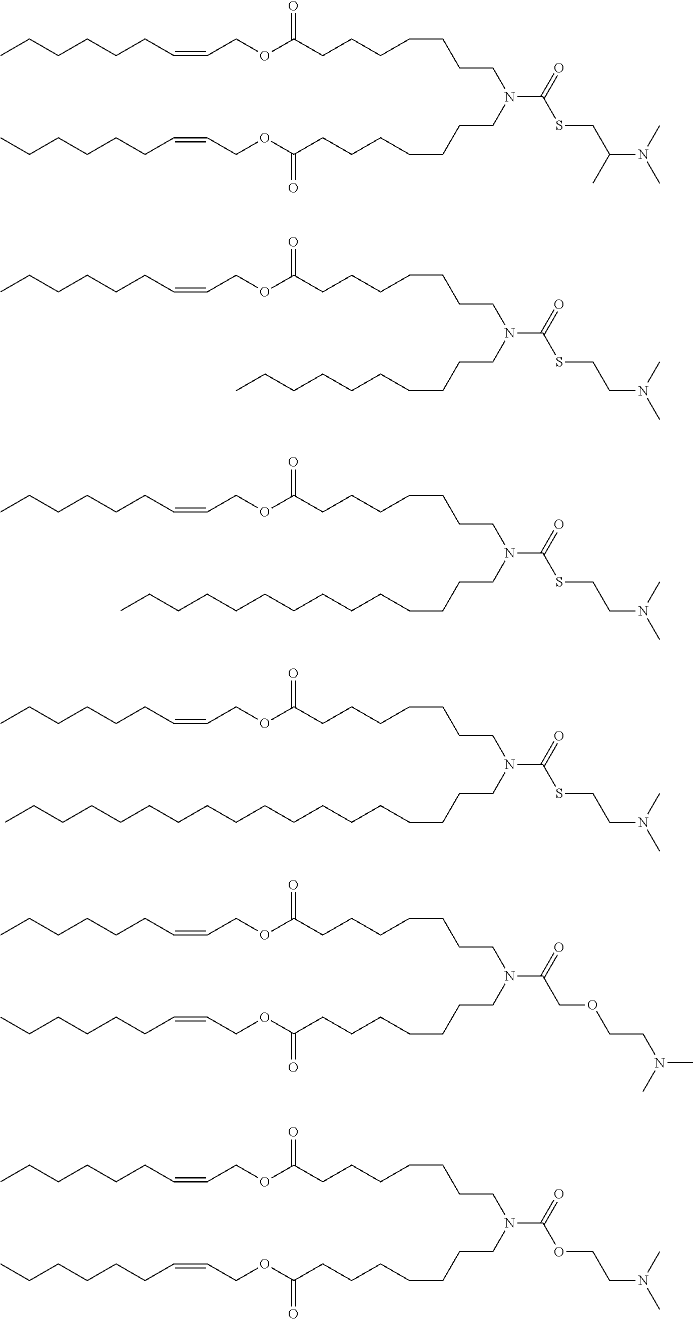

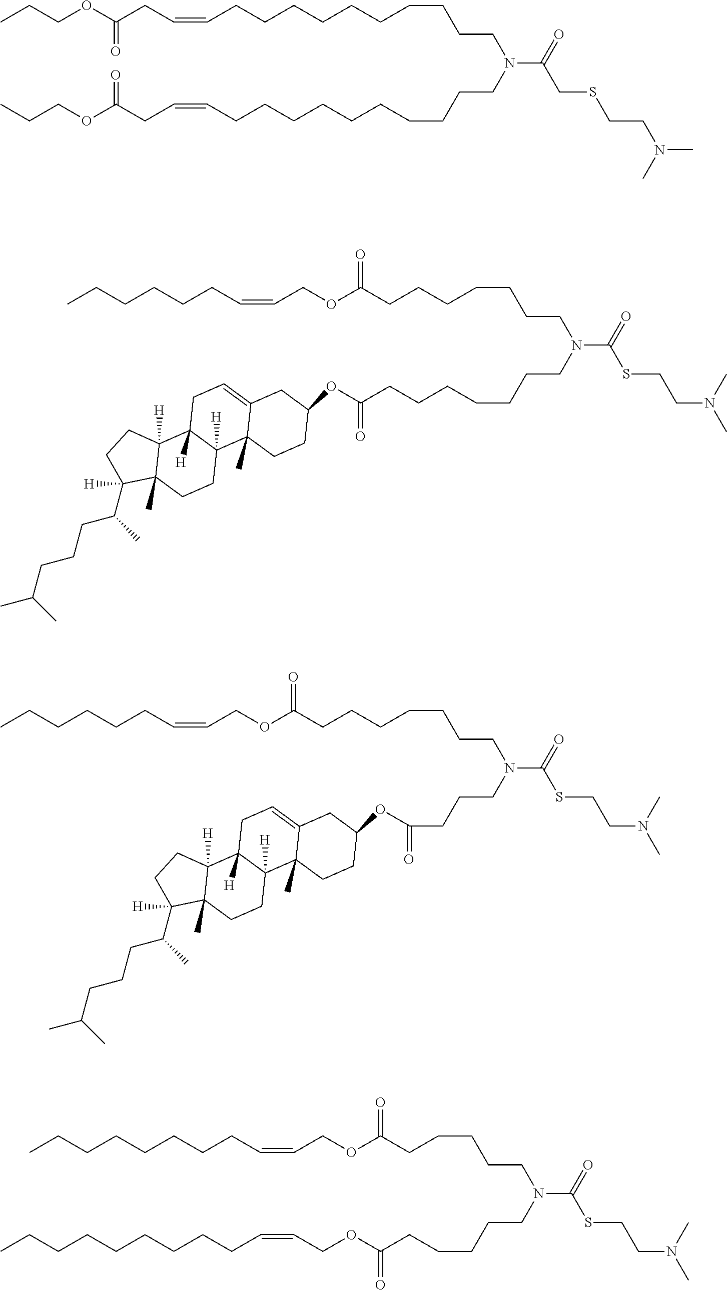

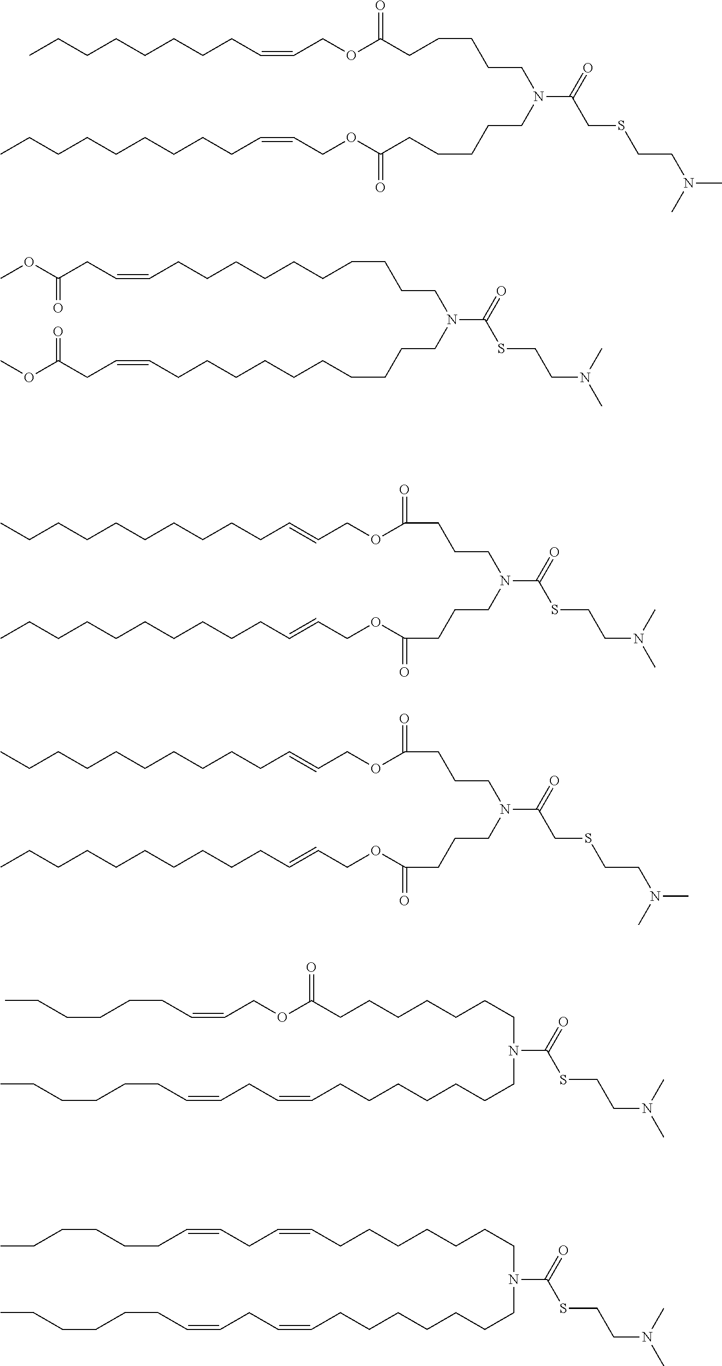

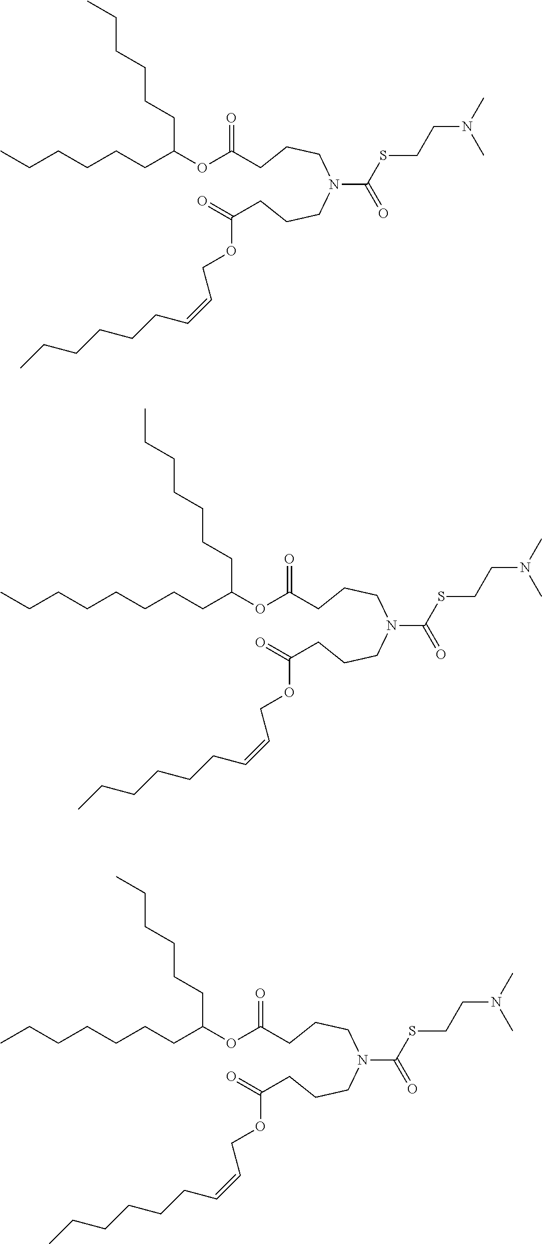

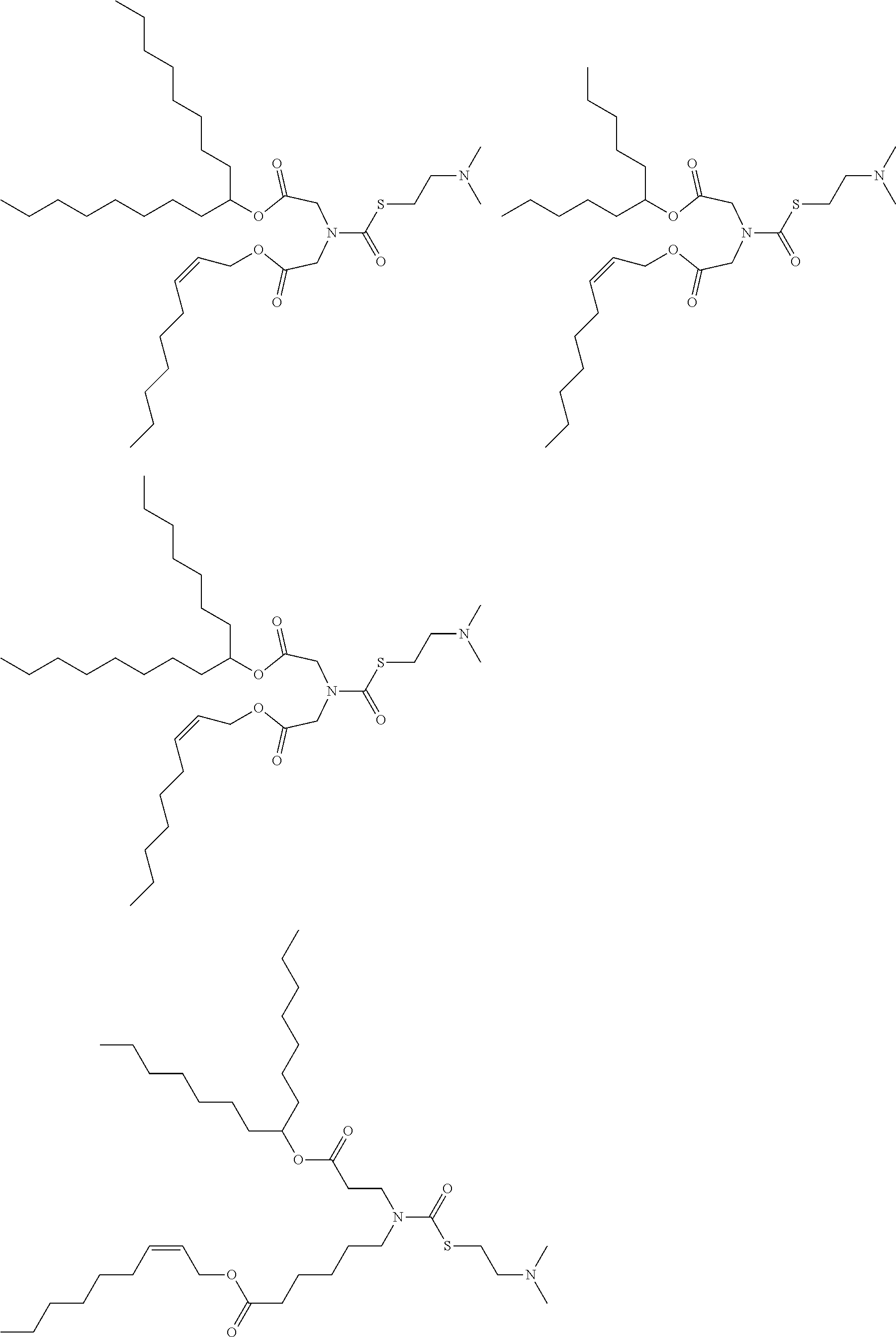

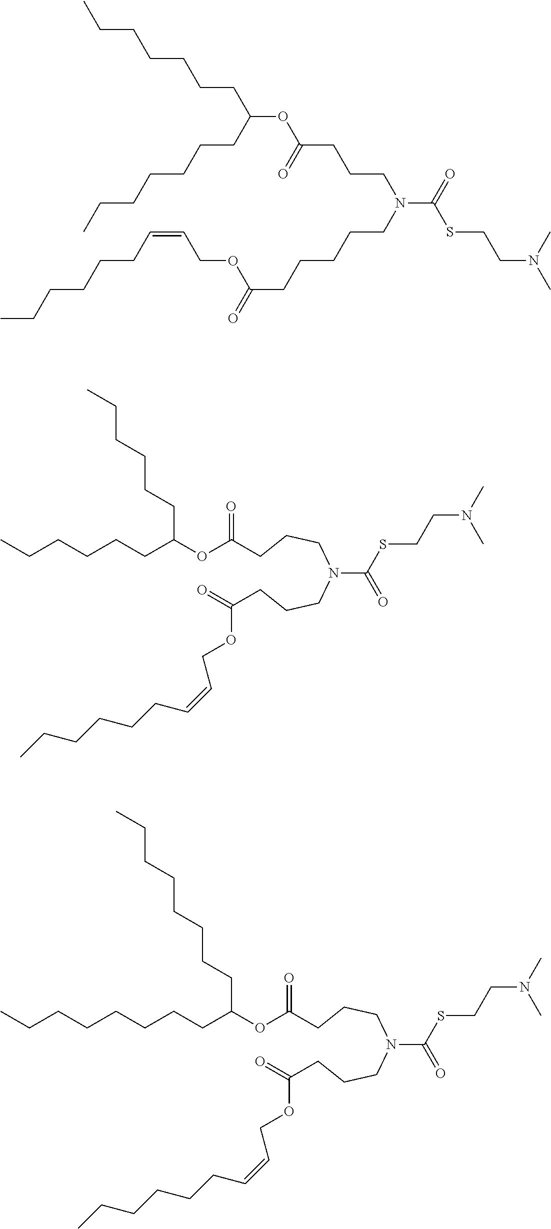

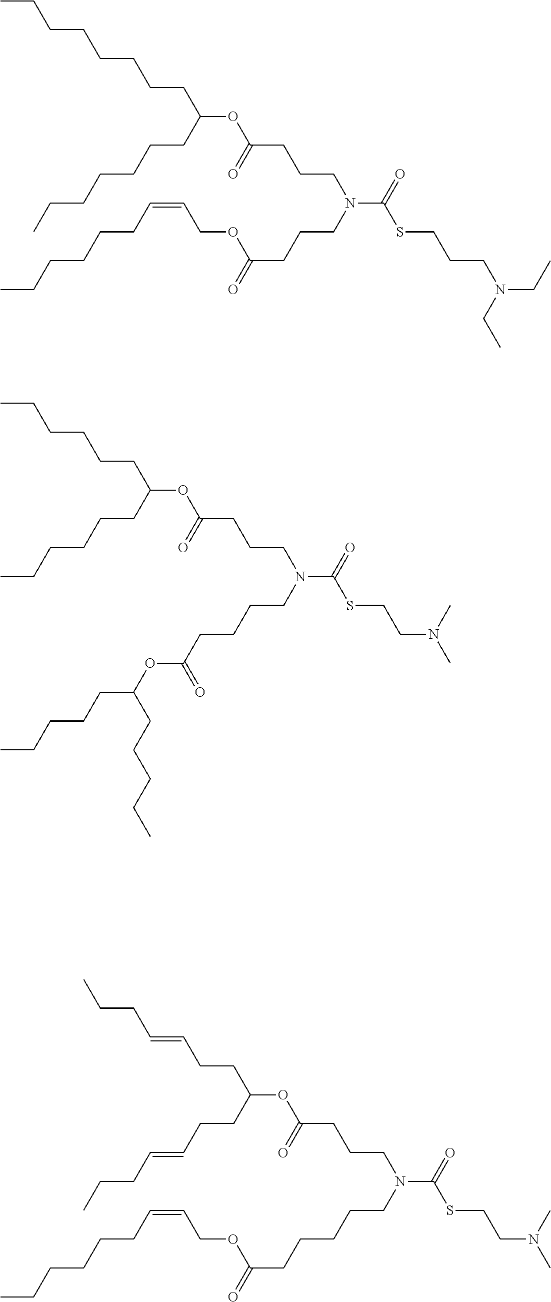

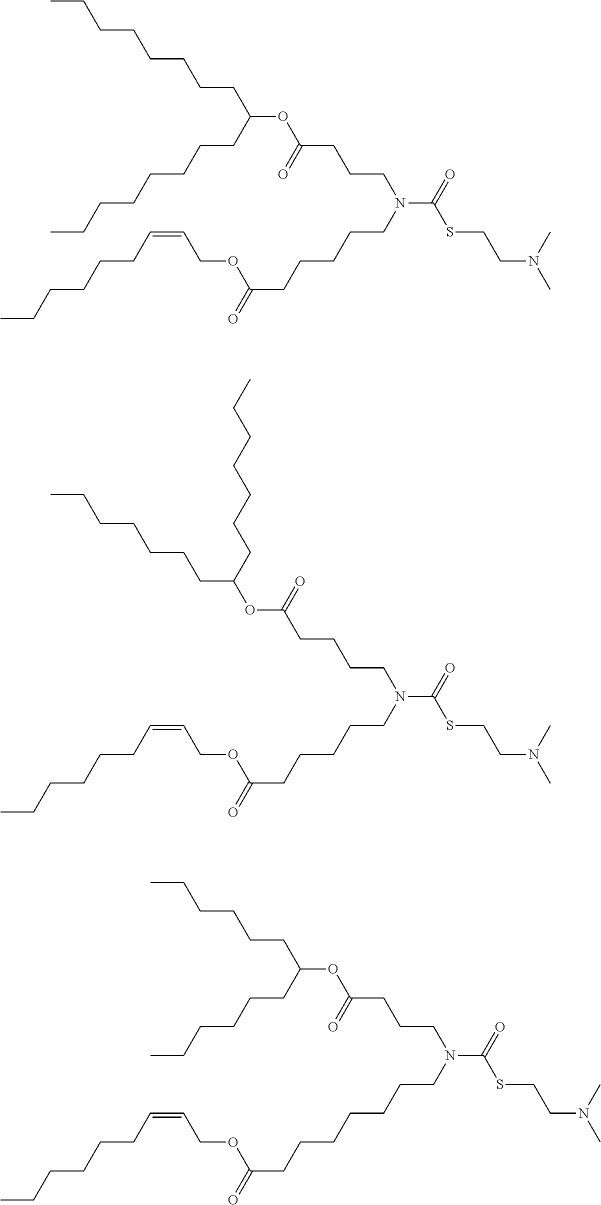

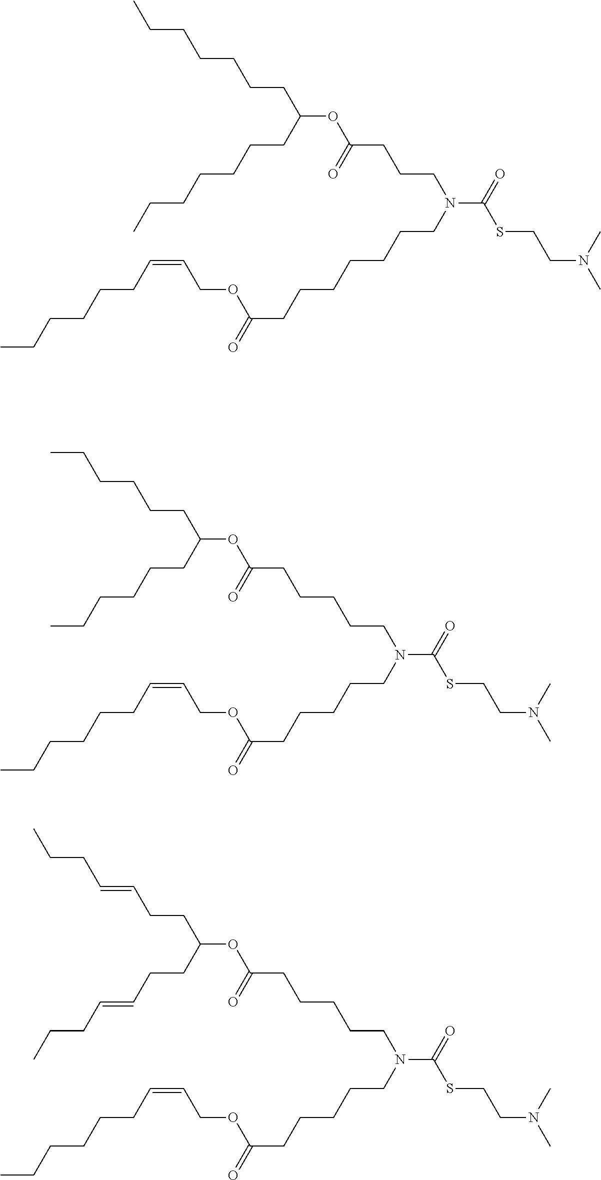

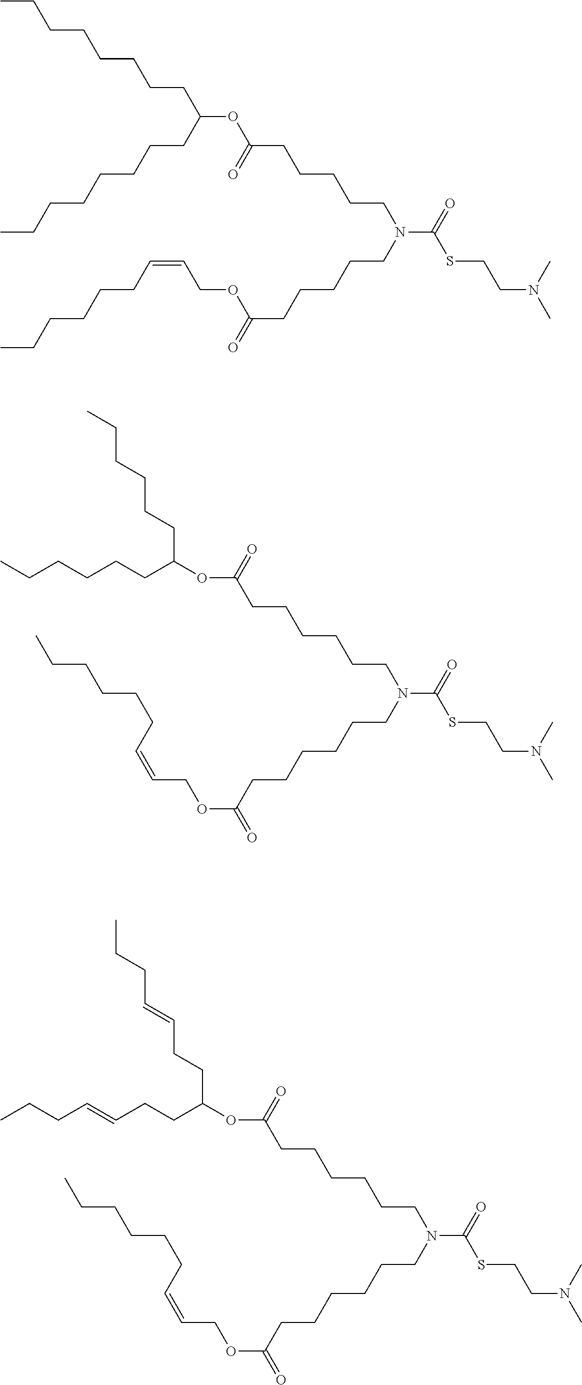

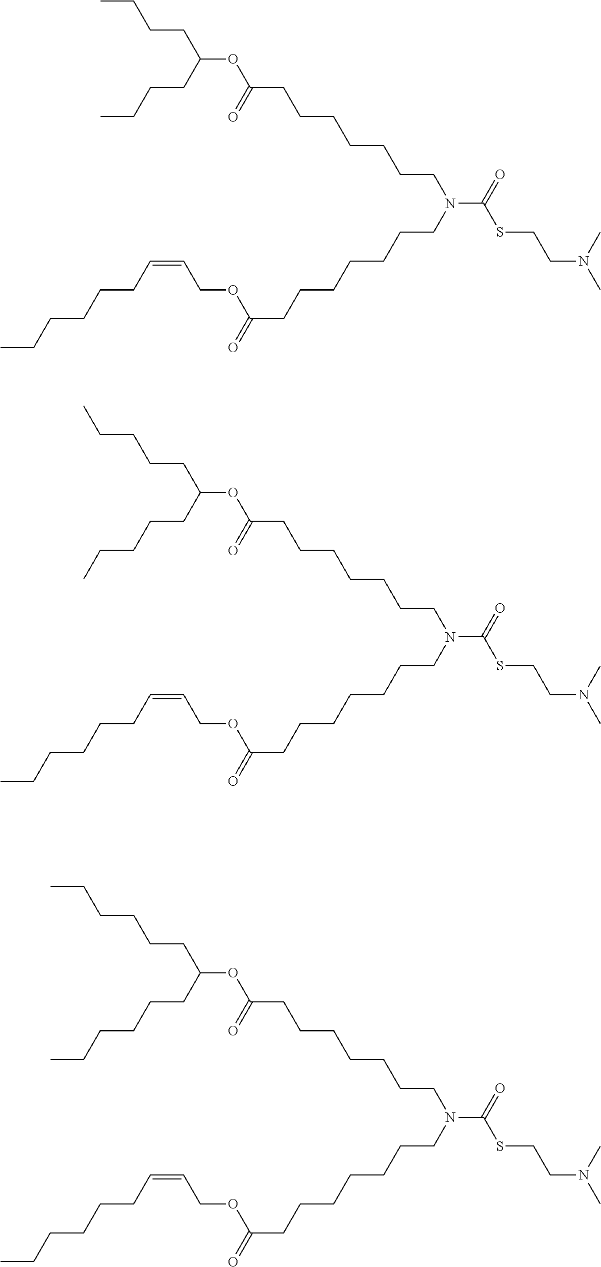

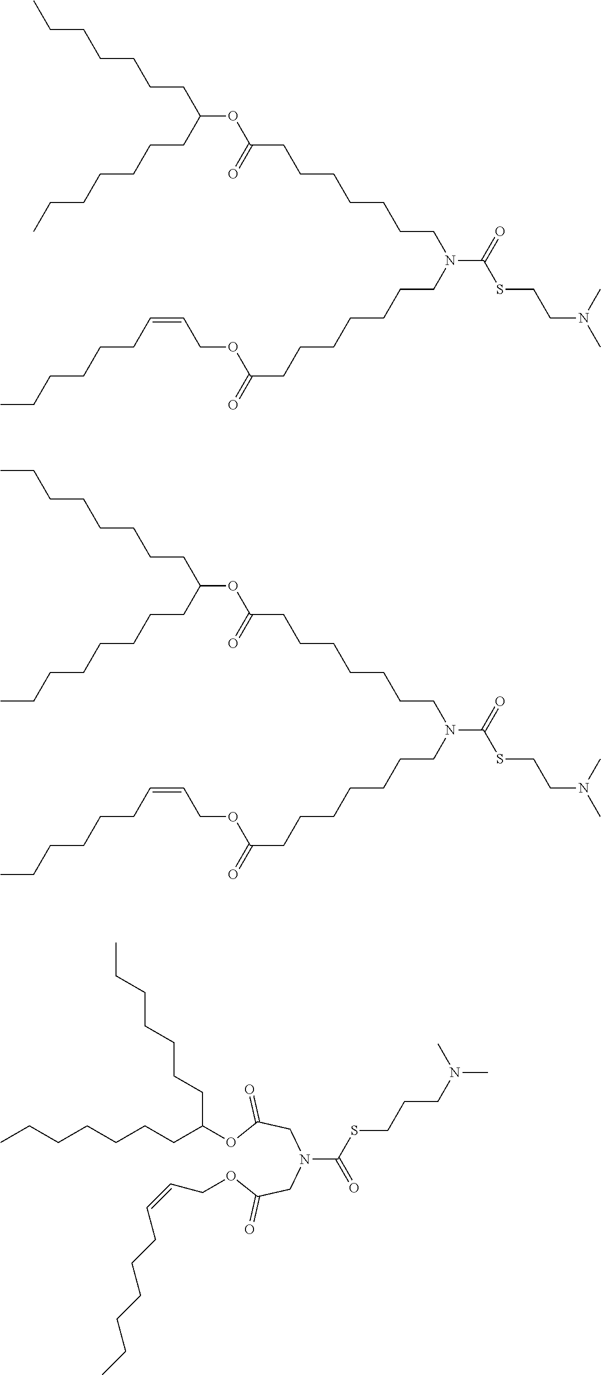

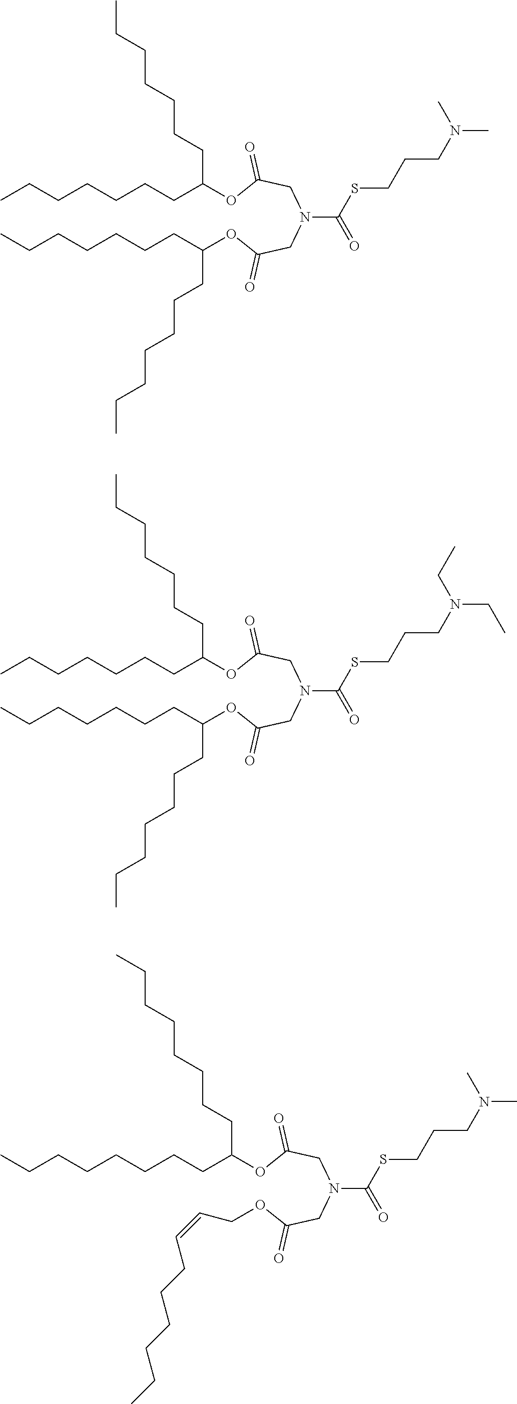

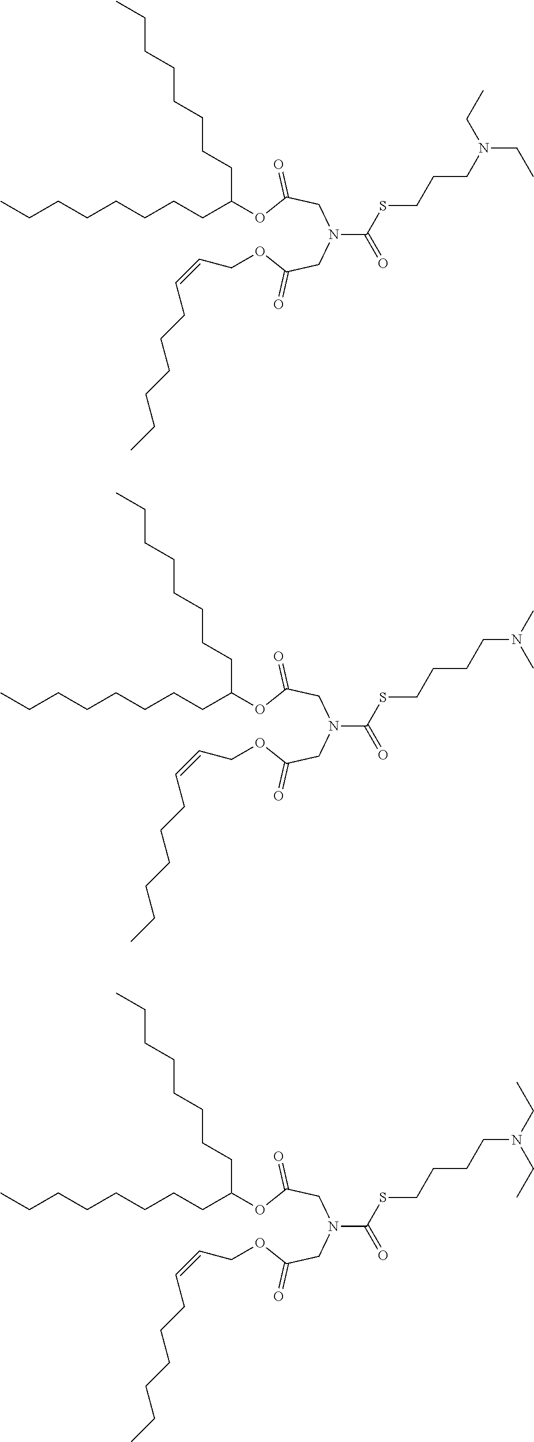

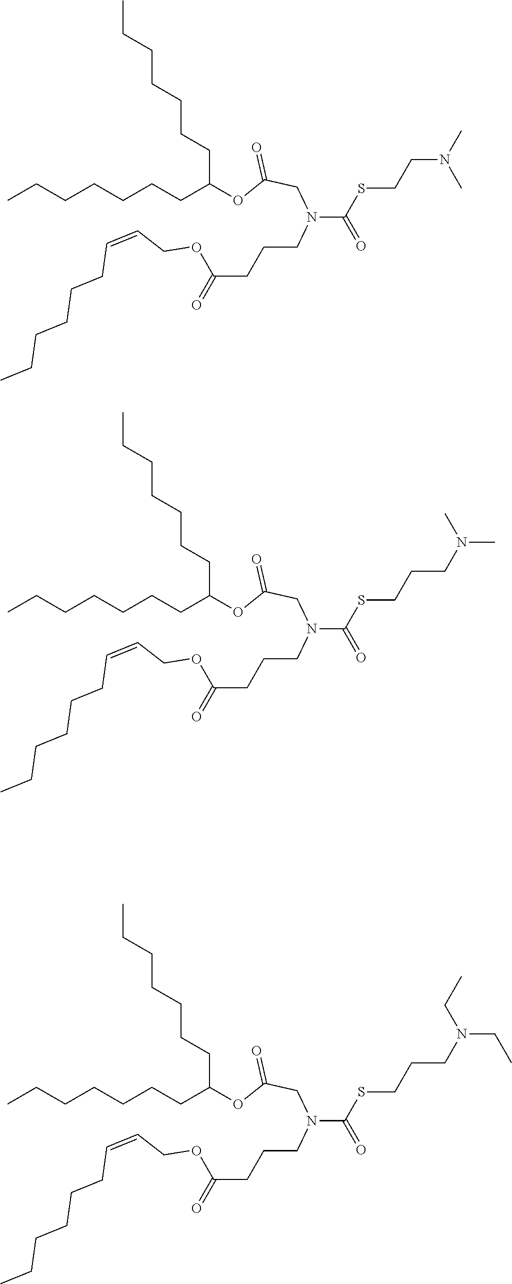

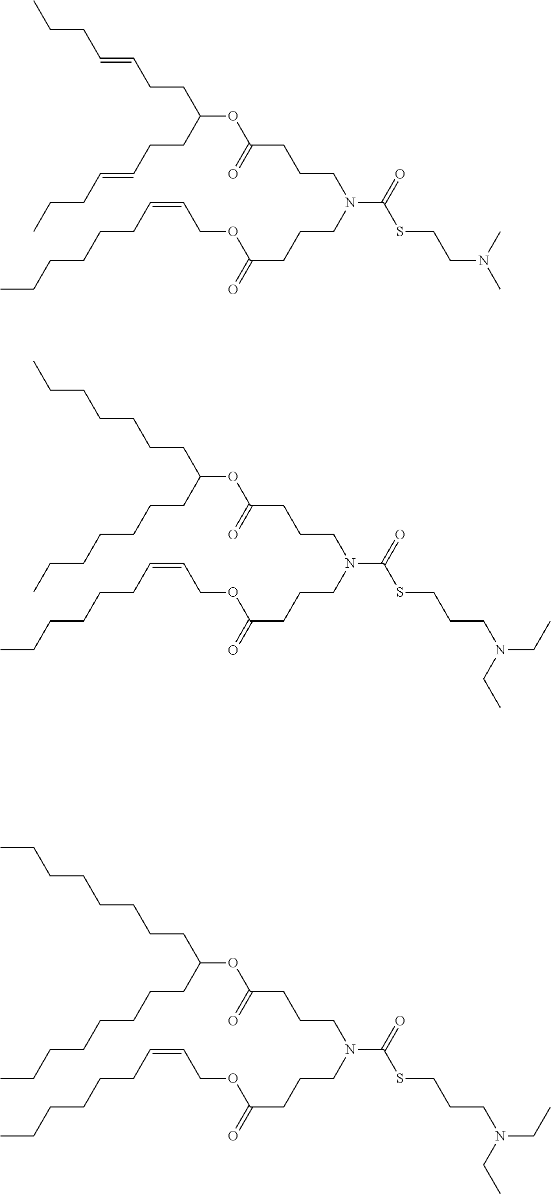

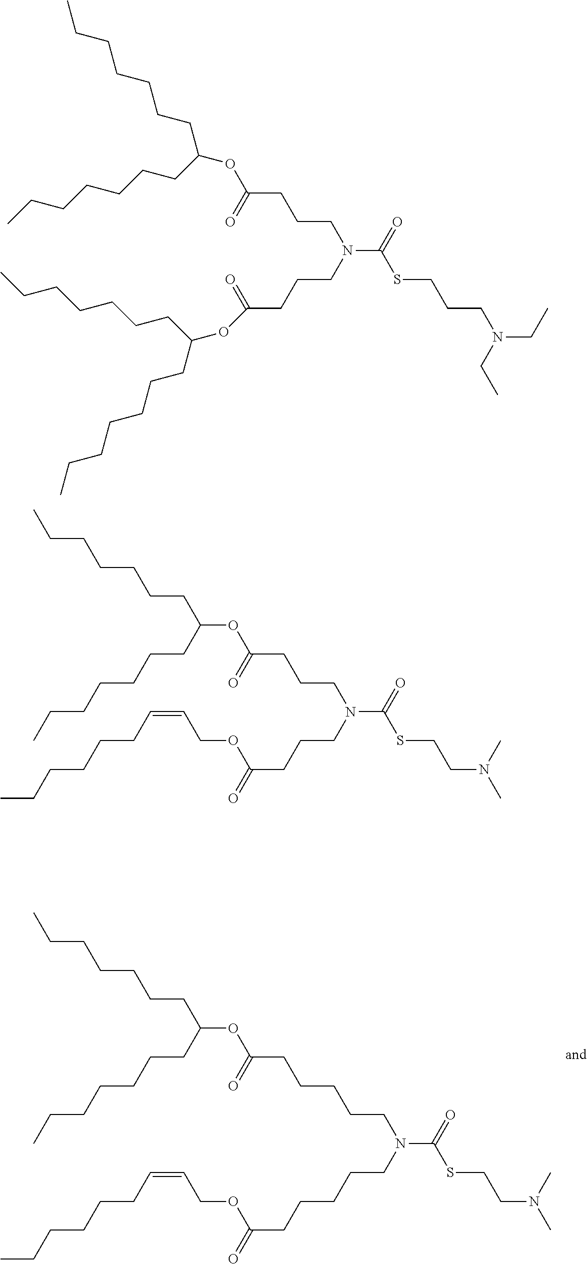

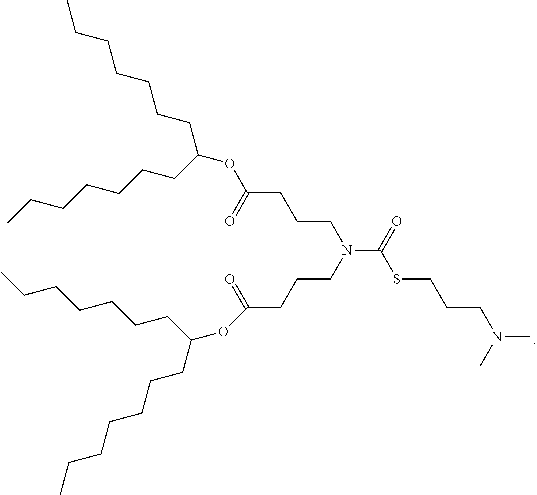 [SMILES: CCCCCCCC(CCCCCCC)OC(=O)CCCN(CCCC(=O)OC(CCCCCCC)CCCCCCC)C(=O)SCCCN(C)C]

In embodiments, any one or more lipids recited herein may be expressly excluded.

### Helper Lipids and Sterols

The UNA oligomer-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the neutral lipid present in the lipid formulation comprises or consists of one or more phospholipids, e.g., a cholesterol-free lipid formulation. In yet other embodiments, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

The percentage of helper lipid present in the lipid formulation is a target amount, and the actual amount of helper lipid present in the formulation may vary, for example, by +5 mol %.

A lipid formulation containing a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some embodiments, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps to protects nanoparticles from the immune system and their escape from RES uptake (Nanomedicine (Lond). 2011 June; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). It has been shown that increased PEGylation leads to a significant increase in the circulation half-life of lipid formulations (Annu. Rev. Biomed. Eng. 2011 Aug. 15; 13( ):507-30; J. Control Release. 2010 Aug. 3; 145(3):178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate can comprise a mixture of a compound of Formula II, III, and or IV as described herein in combination with a PEG-lipid. In some embodiments, the lipid conjugate can comprise a lipid having one or more GalNAc moieties conjugated thereto.

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a trinucleotide expansion interfering UNA oligomer-lipid formulation described herein, the UNA oligomer-lipid formulation enters cells through receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For lipid formulations comprising a GalNAc moiety, Tris-GalNAc binds to the Asialoglycoprotein receptor that is highly expressed on hepatocytes resulting in rapid endocytosis. While the exact mechanism of escape across the endosomal lipid bilayer membrane remains unknown, sufficient amounts of siRNAs enter the cytoplasm to induce robust, target selective RNAi responses in vivo.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Methods for Treating or Preventing Metabolic Disorders

Provided herein, in some embodiments, are methods of treating or preventing a metabolic disorder or condition in a subject. Methods of treating or preventing metabolic disorders or conditions provided herein include administering to a subject an amount of a compound or pharmaceutical composition provided herein effective for treating the metabolic disorder or condition.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. As used herein, the term “effective amount” or “therapeutically effective amount” refers to that amount of a nucleic acid molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell or target organ. The specific dose will vary depending on the particular nucleic acid molecule, composition, or pharmaceutical composition chosen, the dosing regimen to be followed, whether it is administered in combination with other compounds, timing of administration, the tissue to which it is administered, and the physical delivery system in which it is carried.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a disease, disorder, or medical condition, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Exemplary doses of compositions and pharmaceutical compositions that can be administered in the methods provided herein include at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight, and any number or range in between. In some aspects, compositions and pharmaceutical compositions are administered to a subject at a dose of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 200 mg, at least about 250 mg, at least about 300 mg, at least about 350 mg, at least about 400, at least about 450 mg, at least about 500 mg, or more, and any number or range in between.

Compositions and pharmaceutical compositions can be administered in a single dose or as part of a single treatment cycle. Compositions and pharmaceutical compositions provided herein can also be administered multiple times. For example, compositions and pharmaceutical compositions provided herein can be administered once, twice, three times, four times, five times, six times, seven times, eight times, nine times, or more. Timing between two or more administrations can be one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between.

Any metabolic disorder or condition can be treated or prevented by administering a composition or pharmaceutical composition provided herein. Exemplary metabolic disorders and conditions include obesity, liver steatosis, obesity-associated liver steatosis, liver inflammation, liver fibrosis, cardiovascular disease, atherosclerosis, coronary artery disease, diabetes, prediabetes, obesity-associated fat accretion, obesity-related adipose tissue dysfunction, and others. In one aspect, administering a compound or pharmaceutical composition provided herein decreases liver lipid accumulation, decreases liver triglyceride levels, decreases lipogenic and oxidative stress gene expression, decreases insulin resistance, reduces glycemia, or any combination thereof, as compared with administering a control compound or vehicle, or as compared with not administering the compound or pharmaceutical composition. In another aspect, administering a compound or pharmaceutical composition provided herein increases food consumption, reduces fat mass gain, increases circulating adiponectin levels, increases expression of adipogenic and thermogenic genes in adipose tissue, or any combination thereof, as compared with administering a control compound or vehicle, or as compared with not administering the compound or pharmaceutical composition. In one aspect, administering a compound or pharmaceutical composition provided herein improves a cardiovascular condition. As an example, increased adiponectin levels can improve cardiovascular outcomes. Accordingly, increased adiponectin levels seen upon administration of compounds and pharmaceutical compositions provided herein can improve cardiovascular conditions and cardiovascular outcomes. In yet another aspect, administering a compound or pharmaceutical composition provided herein increases food consumption, reduces fat mass gain, increases expression of adipogenic and thermogenic genes in adipose tissue, or any combination thereof, as compared with administering a control compound or vehicle, or as compared with not administering the compound or pharmaceutical composition. In one aspect, increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In a further aspect, adipose tissue with increased expression of adipogenic and thermogenic genes upon administration of a compound or pharmaceutical composition provided herein is white adipose tissue.

Compounds and pharmaceutical compositions of methods provided herein can be administered in any suitable manner. Exemplary methods of administration include intravenous, oral, sublingual, intramuscular, subcutaneous, intradermal, transdermal, intranasal, intraperitoneal, topical, or pulmonary administration. In some aspects, compounds provided herein include an siRNA conjugated to N-acetylgalactosamine (GalNAc; e.g., siLBP-GalNAc). As used herein, an siRNA conjugated to GalNAc is referred to as a “naked siRNA-GalNAc-conjugated” compound or as “siRNA-GalNAc.” Any GalNAc conjugate can be used. Exemplary siRNA conjugates include a GalNAc trimer. Any suitable method can be used to administer siLBP-GalNAc compounds provided herein, including intravenous, oral, sublingual, intramuscular, subcutaneous, intradermal, transdermal, intranasal, intraperitoneal, topical, or pulmonary administration. In one aspect, siLBP-GalNAc compounds provided herein are administered intradermally or transdermally.

Compositions and pharmaceutical compositions can be administered to any subject in the methods provided herein. In one aspect, the subject suffers from the metabolic disorder or condition that is treated or prevented using the methods provided herein. In another aspect, the subject is a healthy subject. As used herein, the term “healthy subject” refers to a subject that does not suffer from the metabolic disease or condition that is treated or prevented using the methods provided herein. Accordingly, a healthy subject may not suffer from any disease or condition or may suffer from a disease or condition other than the metabolic disorder or condition being treated or prevented using the methods provided herein. In other aspects, the subject is an obese subject or a non-obese subject. In one aspect, the subject consumes a high fat diet.

### Methods for Treating or Preventing LBP-Mediated Disorders

Provided herein, in some embodiments, are methods of treating an LBP-mediated disorder or condition in a subject. Methods of treating or preventing LBP-mediated disorders or conditions provided herein include administering to a subject an amount of a compound or pharmaceutical composition provided herein effective for treating the metabolic disorder or condition.

Exemplary doses of compositions and pharmaceutical compositions that can be administered in the methods provided herein include at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight, and any number or range in between. In some aspects, compositions and pharmaceutical compositions are administered to a subject at a dose of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 200 mg, at least about 250 mg, at least about 300 mg, at least about 350 mg, at least about 400, at least about 450 mg, at least about 500 mg, or more, and any number or range in between.

Compositions and pharmaceutical compositions can be administered in a single dose or as part of a single treatment cycle. Compositions and pharmaceutical compositions provided herein can also be administered multiple times. For example, compositions and pharmaceutical compositions provided herein can be administered once, twice, three times, four times, five times, six times, seven times, eight times, nine times, or more. Timing between two or more administrations can be one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between.

Any LBP-mediated disorder or condition can be treated or prevented by administering a composition or pharmaceutical composition provided herein. Exemplary LBP-mediated disorders and conditions include obesity, liver steatosis, obesity-associated liver steatosis, liver inflammation, liver fibrosis, cardiovascular disease, atherosclerosis, coronary artery disease, diabetes, prediabetes, obesity-associated fat accretion, obesity-related adipose tissue dysfunction, and others. In one aspect, administering a compound or pharmaceutical composition provided herein decreases liver lipid accumulation, decreases liver triglyceride levels, decreases lipogenic and oxidative stress gene expression, decreases insulin resistance, reduces glycemia, or any combination thereof, as compared with administering a control compound or vehicle, or as compared with not administering the compound or pharmaceutical composition. In another aspect, administering a compound or pharmaceutical composition provided herein increases food consumption, reduces fat mass gain, increases circulating adiponectin levels, increases expression of adipogenic and thermogenic genes in adipose tissue, or any combination thereof, as compared with administering a control compound or vehicle, or as compared with not administering the compound or pharmaceutical composition. In one aspect, administering a compound or pharmaceutical composition provided herein improves a cardiovascular condition. In another aspect, administering a compound or pharmaceutical composition provided herein improves cardiovascular outcomes. In yet another aspect, administering a compound or pharmaceutical composition provided herein increases food consumption, reduces fat mass gain, increases expression of adipogenic and thermogenic genes in adipose tissue, or any combination thereof, as compared with administering a control compound or vehicle, or as compared with not administering the compound or pharmaceutical composition. In one aspect, increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In a further aspect, adipose tissue with increased expression of adipogenic and thermogenic genes upon administration of a compound or pharmaceutical composition provided herein is white adipose tissue.

Compounds and pharmaceutical compositions of methods provided herein can be administered by any suitable manner. Exemplary methods of administration include intravenous, oral, sublingual, intramuscular, subcutaneous, intradermal, transdermal, intranasal, intraperitoneal, topical, or pulmonary administration. In some aspects, compounds provided herein include an siRNA conjugated to N-acetylgalactosamine (GalNAc; e.g., siLBP-GalNAc). As used herein, an siRNA conjugated to GalNAc is referred to as a “naked siRNA-GalNAc-conjugated” compound or as “siRNA-GalNAc.” Any GalNAc conjugate can be used. Exemplary siRNA conjugates include a GalNAc trimer. Any suitable method can be used to administer siLBP-GalNAc compounds provided herein, including intravenous, oral, sublingual, intramuscular, subcutaneous, intradermal, transdermal, intranasal, intraperitoneal, topical, or pulmonary administration. In one aspect, siLBP-GalNAc compounds provided herein are administered intradermally or transdermally.

Compositions and pharmaceutical compositions can be administered to any subject in the methods provided herein. In one aspect, the subject suffers from the LBP-mediated disorder or condition that is treated or prevented using the methods provided herein. In another aspect, the subject is a healthy subject. In yet another aspect, the subject is an obese subject or a non-obese subject. In one aspect, the subject consumes a high fat diet.

### Methods of Inhibiting LBP Expression

Provided herein, in some embodiments, are methods of inhibiting expression of LBP in a subject. Methods of inhibiting expression of LBP provided herein include administering to a subject an amount of a compound or pharmaceutical composition provided herein effective for inhibiting LBP expression. In another aspect, methods of inhibiting expression of LBP provided herein include administering to a subject an amount of any compound or pharmaceutical composition provided herein effective for inhibiting LBP expression, thereby treating a metabolic disorder or condition in the subject.

In one aspect, LBP expression is selectively inhibited in the liver, in plasma, or in both the liver and in plasma. LBP expression can be reduced by about 0.1%, about 0.5%, about 1%, about 2.5%, about 5%, about 10%, about 20%, about 30%, about 40%, about 50%, about 60%, about 70%, about 80%, about 90%, about 95%, about 96%, about 97%, about 98%, about 99%, about 99.5%, about 100%, and any number or range in between, as compared to expression in the absence of a compound or pharmaceutical composition provided herein or as compared to the presence of a control compound. LBP expression can also be reduced about one-fold, about two-fold, about three-fold, about four-fold, about five-fold, about six-fold, about seven-fold, about eight-fold, about nine-fold, about ten-fold, about 20-fold, about 30-fold, about 40-fold, about 50-fold, about 60-fold, about 70-fold, about 80-fold, about 90-fold, about 100-fold, or more, and any number or range in between, as compared to expression in the absence of a compound or pharmaceutical composition provided herein or as compared to the presence of a control compound. In an aspect, there is no LBP expression. In another aspect, there is no detectable LBP expression. In one aspect, liver LBP expression is inhibited. In another aspect, plasma LBP expression is inhibited. In yet another aspect, both liver LBP and plasma LBP expression are inhibited.

Exemplary doses of compositions and pharmaceutical compositions that can be administered in the methods provided herein include at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight, and any number or range in between. In some aspects, compositions and pharmaceutical compositions are administered to a subject at a dose of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 200 mg, at least about 250 mg, at least about 300 mg, at least about 350 mg, at least about 400 mg, at least about 450 mg, at least about 500 mg, or more, and any number or range in between.

Compositions and pharmaceutical compositions can be administered in a single dose or as part of a single treatment cycle. Compositions and pharmaceutical compositions provided herein can also be administered multiple times. For example, compositions and pharmaceutical compositions provided herein can be administered once, twice, three times, four times, five times, six times, seven times, eight times, nine times, or more. Timing between two or more administrations can be one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between.

Methods of inhibiting LBP expression may be used to treat a metabolic disorder or condition. In some aspects, the metabolic disorder or condition is obesity, liver steatosis, obesity-associated liver steatosis, liver inflammation, liver fibrosis, cardiovascular disease, atherosclerosis, coronary artery disease, diabetes, prediabetes, obesity-associated fat accretion, or obesity-related adipose tissue dysfunction. In one aspect, administering a compound or pharmaceutical composition provided herein decreases liver lipid accumulation, decreases liver triglyceride levels, decreases lipogenic and oxidative stress gene expression, decreases insulin resistance, reduces glycemia, or any combination thereof, as compared with administering a control compound or vehicle, or as compared with not administering the compound or pharmaceutical composition. In another aspect, administering a compound or pharmaceutical composition provided herein increases food consumption, reduces fat mass gain, increases circulating adiponectin levels, increases expression of adipogenic and thermogenic genes in adipose tissue, or any combination thereof, as compared with administering a control compound or vehicle, or as compared with not administering the compound or pharmaceutical composition. In one aspect, administering a compound or pharmaceutical composition provided herein improves a cardiovascular condition. In another aspect, administering a compound or pharmaceutical composition provided herein improves cardiovascular outcomes. In yet another aspect, administering a compound or pharmaceutical composition provided herein increases food consumption, reduces fat mass gain, increases expression of adipogenic and thermogenic genes in adipose tissue, or any combination thereof, as compared with administering a control compound or vehicle, or as compared with not administering the compound or pharmaceutical composition. In one aspect, increased food consumption, reduced fat mass gain, and increased expression of adipogenic and thermogenic genes in adipose tissue is sex-specific. In another aspect, increased food consumption, reduced fat mass gain, and increased expression of adipogenic and thermogenic genes in adipose tissue is female-specific. In a further aspect, adipose tissue with increased expression of adipogenic and thermogenic genes upon administration of a compound or pharmaceutical composition provided herein is white adipose tissue.

Compounds and pharmaceutical compositions of methods provided herein can be administered by any suitable manner. Exemplary methods of administration include intravenous, oral, sublingual, intramuscular, subcutaneous, intradermal, transdermal, intranasal, intraperitoneal, topical, or pulmonary administration. In some aspects, compounds provided herein include an siRNA conjugated to N-acetylgalactosamine (GalNAc; e.g., siLBP-GalNAc). As used herein, an siRNA conjugated to GalNAc is referred to as a “naked siRNA-GalNAc-conjugated” compound or as “siRNA-GalNAc.” Any GalNAc conjugate can be used. Exemplary siRNA conjugates include a GalNAc trimer. Any suitable method can be used to administer siLBP-GalNAc compounds provided herein, including intravenous, oral, sublingual, intramuscular, subcutaneous, intradermal, transdermal, intranasal, intraperitoneal, topical, or pulmonary administration. In one aspect, siLBP-GalNAc compounds provided herein are administered intradermally or transdermally.

Compositions and pharmaceutical compositions can be administered to any subject in the methods provided herein. In one aspect, the subject suffers from the LBP-mediated disorder or condition that is treated or prevented using the methods provided herein. In another aspect, the subject is a healthy subject. In yet another aspect, the subject is an obese subject or a non-obese subject. In one aspect, the subject consumes a high fat diet.

### Materials and Methods for Examples 1-8

### Human Study

Three hundred and sixty-three subjects were recruited from the ongoing multicenter FLORINASH Project, with a body mass index (BMI) >35 kg/m². Participants were recruited at the Endocrinology Service of the Hospital Universitari Dr. Josep Trueta (Girona, Spain). Anthropometric and clinical parameters are described in Table 1. Inclusion criteria were age from 30 to 65 years and ability to understand study procedures. Exclusion criteria were systemic diseases, infection in the previous month, serious chronic illness, >20 g ethanol intake/day, or use of medications that might interfere with insulin action. In a subgroup of 58 participants (Table 2), liver and subcutaneous (SAT) and visceral (VAT) adipose tissue samples were snap frozen in liquid nitrogen for gene expression analysis. Liver samples were also fixed in formalin for histological assessment. Fixed samples were stained with hematoxylin-eosin and Masson's trichrome stain. All samples were evaluated by the same pathologist according to degree of steatosis. Then, participants were stratified as subjects without significant steatosis (<5%), “borderline” (5-33%), and subjects with significant steatosis (>33% of fat). Exclusion criteria included cirrhosis or bridging fibrosis, a liver biopsy less than 2 cm long, and use of statins. This study was carried out in accordance with the recommendations of the ethical committee of the Hospital of Girona “Dr Josep Trueta.” The protocol was approved by the ethical committee of the Hospital of Girona “Dr Josep Trueta.” All subjects gave written informed consent in accordance with the Declaration of Helsinki, after the purpose of the study was explained.

TABLE 1
Anthropometric and clinical parameters in whole cohort

| | | | | | Moderate | | | | |
|---|---|---|---|---|---|---|---|---|---|
| | No steatosis | | Slight steatosis | | steatosis | | Severe steatosis | | p value |
| N | 147 | | 94 | | 81 | | 41 | | |
| Gender (men/women) | 23/124 | | 26/68 | | 33/48 | | 23/18 | | |
| Age (years) | 42.8 ± 9.5 | | 45.7 ± 9 | | 47.5 ± 8.3^{a} | | 46.2 ± 9.1 | | 0.002 |
| BMI (Kg/m2) | 43.7 ± 5.7 | | 44.4 ± 5.7 | | 44.9 ± 5.9 | | 46.5 ± 5.3^{a} | | 0.04 |
| Waist circumference (cm) | 120.3 ± 13.3 | | 124.2 ± 14.8 | | 128.4 ± 13.1^{a} | | 133.6 ± 9.4^{ab} | | <0.0001 |
| HOMA_{IR}* | 2.4 | (1.5-3.6) | 3.5 | (2.2-4.8) | 4.4 | (2.9-6.2)^{a} | 4.6 | (3.1-7.1) | 0.01 |
| Fasting glucose (mg/dl) | 90.5 ± 12.2 | | 98.7 ± 27.1^{a} | | 101.1 ± 23.4^{a} | | 103.4 ± 18.8^{a} | | <0.0001 |
| HbA1c (%) | 5.5 ± 0.4 | | 5.8 ± 0.7^{a} | | 5.8 ± 0.8^{a} | | 6.1 ± 0.6^{a} | | <0.0001 |
| Total cholesterol (mg/dl) | 192.4 ± 36.9 | | 192.6 ± 31.5 | | 199.4 ± 35.2 | | 199.3 ± 36.6 | | 0.3 |
| HDL-cholesterol (mg/dl) | 49.1 ± 11.5 | | 47.9 ± 9.9 | | 44.7 ± 12.7^{a} | | 42.3 ± 6.5ab | | 0.001 |
| LDL-cholesterol (mg/dl) | 120 ± 30.3 | | 119.9 ± 29.2 | | 125.7 ± 28.4 | | 124.5 ± 31.7 | | 0.4 |
| Fasting triglycerides (mg/dl)* | 102 | (78-142) | 112.5 | (83.5-156) | 123 | (98-170.5)^{a} | 160 | (106.5-213.5)^{ab} | <0.0001 |
| hsCRP (mg/dl)* | 0.63 | (0.34-1.01) | 0.85 | (0.43-1.25) | 0.65 | (0.42-1.20) | 0.98 | (0.42-1.63)^{a} | 0.03 |

| | | | | | |
|---|---|---|---|---|---|
| Plasma LBP (μg/ml) | 27.7 ± 10.8 | 28.1 ± 11.1 | 30.9 ± 12.1 | 33.6 ± 11.9ab | 0.009 |

HOMA_{IR}, homeostasis model assessment − insulin resistance index.
*median and interquartile range.
^{a}p < 0.05 compared to morbidly obese participants without liver steatosis after performing Bonferroni post hoc test.
^{b}p < 0.05 compared to morbidly obese participants with slight liver steatosis after performing Bonferroni post hoc test.

TABLE 2
Anthropometric and clinical parameters in morbidly
obese participants with hepatic pathology analysis.

| | No NAFLD | | Borderline | | NAFLD | | p value |
|---|---|---|---|---|---|---|---|
| N | 18 | | 24 | | 16 | | |
| Gender (men/women) | 2/16 | | 5/19 | | 4/12 | | |
| Age (years) | 45.1 ± 9.3 | | 47.3 ± 8.9 | | 49.9 ± 6.4 | | 0.2 |
| BMI (Kg/m2) | 44.02 ± 7.8 | | 44.81 ± 5.4 | | 44.46 ± 6.8 | | 0.6 |
| Waist circumference (cm) | 120.8 ± 10.4 | | 128.8 ± 11.9 | | 124.8 ± 11.5 | | 0.1 |
| HOMA_{IR}* | 2.2 | (1.5-3.2) | 3.9 | (2.6-8.2) | 4.7 | (3.2-6.3) | 0.1 |
| Fasting glucose (mg/dl)* | 96 | (87-106) | 96 | (89-106) | 100 | (92-129.5)^{a} | 0.04 |
| HbA1c (%) | 5.57 ± 0.5 | | 6.01 ± 1.2 | | 6.49 ± 1.4^{a} | | 0.05 |
| Total cholesterol (mg/dl) | 180.8 ± 29.9 | | 203.5 ± 35.9 | | 186.3 ± 27.8 | | 0.05 |
| HDL-cholesterol (mg/dl) | 47.9 ± 10.9 | | 46 ± 9.1 | | 48.1 ± 15.1 | | 0.8 |
| LDL-cholesterol (mg/dl) | 114.3 ± 25.4 | | 130.9 ± 33.5 | | 108.9 ± 22.7^{b} | | 0.03 |
| Fasting triglycerides (mg/dl)* | 82 | (68-120) | 132 | (99-177)^{a} | 136 | (117.5-163)^{a} | 0.003 |
| hsCRP (mg/dl)* | 0.54 | (0.30-1.01) | 0.74 | (0.59-1.14) | 0.92 | (0.34-1.35) | 0.3 |

*median and interquartile range.
^{a}p < 0.05 compared to morbidly obese participants without NAFLD after performing Bonferroni post hoc test.
^{b}p < 0.05 compared to borderline morbidly obese participants after performing Bonferroni post hoc test.

In Vitro Experiments for Lbp siRNA Selection

Silencer Select Pre-designed siRNA assays si69107, si69108 and si69109 targeting mouse Lbp mRNA were purchased from Ambion, Life Technologies. siRNA sequences were chemically modified by introducing 2′-O-methyl RNA bases, phosphorothioate linkages, and UNA to generate UNA siRNA (Integrated DNA Technologies, USA). Hepa1-6 cells were seeded on 96 well plates the day before transfection at a density of 10⁴ cells per well. Following the vendor recommended protocol, Lipofectamine RNAiMax was used to transfect siRNAs at different concentrations. After 48 hours, cells were washed with PBS and EZCt Cells2Ct Direct Lysis Buffer (Lifeome, USA) was added following the manufacturer's suggested protocol.

### Preparation of Lipid Formulated-Lbp UNA-siRNA

Using lipid nanoparticle (LNP) technology, LNPs encapsulating Lbp-UNA siRNA were produced as described previously¹⁸. Briefly, UNA siRNA was dissolved in 2 mM citrate buffer, pH 3.5. Lipids at the desired molar ratio were dissolved in ethanol. The molar ratio of the constituent lipids was 58% ionizable lipid (Arcturus Therapeutics), 7% DSPC (1,2-distearoyl-sn-glycero-3-phosphocholine) (Avanti Polar Lipids, Alabaster, AL, USA), 33.5% cholesterol (Avanti Polar Lipids, Alabaster, AL, USA), and 1.5% DMG-PEG (1,2-Dimyristoyl-sn-glycerol, methoxypolyethylene glycol, PEG chain Molecular weight: 2000) (NOF America Corporation, White Plains, NY). Lipid solution was then combined with UNA siRNA solution using a Nanoassemblr® microfluidic device (Precision NanoSystems Inc., Vancouver, Canada) at a flow rate ratio of 1:3 ethanol:aqueous phases. The mixed material was then diluted with 3× volume of 10 mM Tris buffer, pH 7.4 containing 9% sucrose, reducing the ethanol content to 6.25%. The diluted formulation was then concentrated by tangential flow filtration using hollow fiber membranes (mPES Kros membranes, 100 Kd MWCO, Spectrum Laboratories, Inc., Rancho Dominguez, California), followed by diafiltration against 10 volumes of 10 mM Tris buffer, pH 7.4 containing 9% sucrose. Post diafiltration, formulations were concentrated to the desired UNA-siRNA concentration followed by filling into vials and freezing. Formulations were characterized for particle size, UNA-siRNA content and encapsulation efficiency. Particle size was determined by dynamic light scattering (ZEN3600, Malvern Instruments). Encapsulation efficiency was calculated by determining unencapsulated UNA-siRNA content by measuring the fluorescence upon the addition of RiboGreen (Molecular Probes) to the particles (Fi) and comparing this value to the total RNA content that is obtained upon lysis of the particles by 1% Triton X-100 (Ft), where % encapsulation=(Ft−Fi)/Ft×100.

### Mouse Experiments

Pilot experiment. Eight week-old male C57/BL6J mice (N=25) were fed with high sucrose and fat diet (HFHS, TD.08811, 4.7 Kcal/g, ENVIGO) for 2 weeks under standard conditions of light (12-hour light/12-hour dark cycle) and temperature (22±1° C.). Next, one tail-intravenous injection of the following treatments were performed: i) vehicle (phosphate buffered saline); ii-iii) chemically unmodified (1 and 3 mg/kg) and iv-v) UNA-containing chemically modified (1 and 3 mg/kg) Lbp siRNA complexed with the LNP delivery platform. The effect of this injection was evaluated at day 3, 6, 10 and 12, and blood was collected in citrate tubes for preparation of plasma each day. The weight of mice was measured at 8 (day −14 of injection) and 10 (day 0 of injections) weeks, and 12 days after injections. Finally, the same mice were injected a second time at week 12, and 3 days later mice were killed by suffocation under sedation, and liver was removed, immediately frozen in liquid-nitrogen, and stored at −80° C. until processing for RNA extraction.

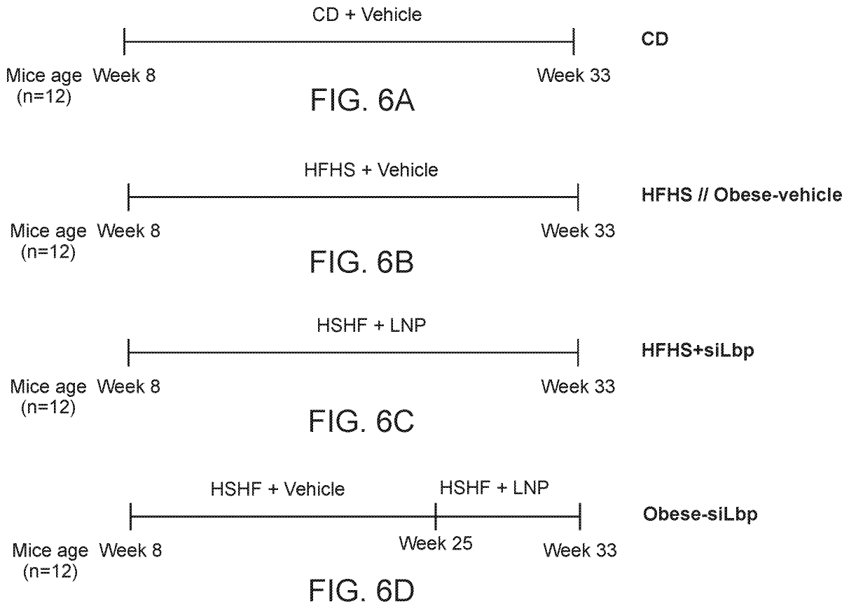

Metabolic experiments. Eight week-old male and female C57/BL6J mice (N=48) were housed for 25 weeks under standard conditions of light (12-hour light/12-hour dark cycle) and temperature (22±1° C.) and under the following experimental conditions: i) Non-treated control diet (CD)-fed mice (TD.120455, 3.3 Kcal/g, ENVIGO) for 25 weeks; ii) Non-treated high sucrose and fat diet (HFHS)-fed mice for 25 weeks; iii) Weekly lipid formulated-Lbp UNA-siRNA (3 mg/kg) treated HFHS-fed mice for 25 weeks (HFHS+L); iv) Weekly lipid formulated-Lbp UNA-siRNA (3 mg/kg) treated HFHS-fed mice only in the last 8 weeks (HFHS+L2) (FIG. 6 A-D). Body weight was reported weekly. Serum and plasma were collected at 4 and 8 weeks. Insulin tolerance test (ITT) was performed at week 20. Briefly, insulin (Actrapid; Novo Nordisk Pharma A/S, Bagsvaerd, Denmark) in saline solution was administered to mice intraperitoneally (0.75 UI/kg), and glycaemia in blood obtained from the tail was measured 15, 30, 45, 60 and 90 minutes after glucose injection. At week 25, after overnight fasting, mice were sacrificed by suffocation under sedation. Then, blood serum and plasma, and liver were collected, immediately frozen in liquid-nitrogen, and stored at −80° C. until processing for RNA or protein analysis. A piece of liver was also fixed with 4% formalin for 24 hours and then stored in 70% ethanol at 4° C. for histological analysis.

In all mouse experiments, the research was conducted in accordance with the European Guidelines for the Care and Use of Laboratory Animals (directive 2010/63/EU) and was approved by the Ethical Committee for Animal Experimentation of Barcelona Science Park (PCB) and University of Barcelona.

### Gene Expression Analysis

RNA purification and gene expression procedures and analyses were carried out as previously described¹⁷. Briefly, RNA purification (isolation) was performed using the RNeasy Lipid Tissue Mini Kit (QIAgen, Izasa SA, Barcelona, Spain) and the integrity was checked by the Agilent Bioanalyzer (Agilent Technologies, Palo Alto, CA). Gene expression was assessed by real-time PCR using a LightCycler® 480 Real-Time PCR System (Roche Diagnostics SL, Barcelona, Spain), using TaqMan® technology suitable for relative gene expression quantification. The commercially available and pre-validated TaqMan® primer/probe sets used were as follows: Endogenous control 18S, and target gene mouse lipopolysaccharide binding protein (Lbp, Mm00493139_m1); fatty acid synthase (Fasn, Mm00662319_m1); stearoyl-Coenzyme A desaturase 1 (Scd1, Mm00772290_m1); acetyl-Coenzyme A carboxylase alpha (Acaca, Mm01304257_m1); sterol regulatory element binding transcription factor 1 (Srebf1, Mm00550338_m1); pyruvate dehydrogenase kinase, isoenzyme 1 (Pdk1, Mm00554300_m1); collagen, type III, alpha 1 (Col3a1, Mm00802300_m1); collagen, type IV, alpha 1 (Col4a1, Mm01210125_m1); transforming growth factor, beta 1 (Tgfb1, Mm01178820_m1); interleukin 6, (Il6, Mm00446190_m1); tumor necrosis factor (Tnf, Mm00443258_m1); integrin alpha X (Itgax, Mm00498701_m1); chemokine (C-C motif) ligand 2 (Ccl2, Mm00441242_m1); interleukin 10 (I110, Mm01288386_m1); glutathione S-transferase, alpha 3 (Gsta3, Mm00494798_m1); glutathione peroxidase 4 (Gpx4, Mm00515041_m1); superoxide dismutase 2, mitochondrial (Sod2, Mm01313000_m1).

### Masson's Trichrome Staining

Tissue processing and a standard Masson's trichrome staining was performed at Allele Biotechnologies (San Diego, California, USA). After staining, slides were examined by a certified pathologist at HistoTox Labs (Bulder, Colorado, USA) who evaluated and quantified the degree of fibrosis. LD area and number were assessed using Fiji (NIH)¹⁹. A minimum of four random regions of interest (ROI) of 151.719 μm² were created from each image and the area and number of the LDs contained in the ROI was retrieved and analyzed.

### Quantification of Triglycerides in Liver.

20-30 mg of liver were homogenized using a TissueLyser LT in 400 μl of distilled water containing 5% Igepal CA-630, boiled twice for 5 minutes, centrifuged at 13,000×g for 5 minutes. Triglycerides were measured in the supernatant using the Serum Triglyceride Determination Kit (TR0100; Sigma-Aldrich). Values were normalized by tissue weights used for the homogenizations.

Lentiviral shRNA-Lbp Particles Production

Four different short-hairpin-Lbp (clone set against mouse Lbp, NM 008489.2) primer sequences and random negative control (NC) sequences that did not have targets for any gene were synthesized by Tebu-bio (Tebu-bio, Spain, SL). Lentivirus-targeted Lbp was obtained by cotransfection of shRNA plasmids against Lbp and a combination of packaging and envelope plasmids from Addgene (pCMV-VSV-G and pCMV-dR8.2 dvpr) into HEK293T using LipoD293 transfection reagent following manufacturers' instructions. Lentiviral particles obtained were used to treat Hepa1-6 cells. Lentiviral effectiveness was confirmed in Hepa1-6 cells.

In Vitro Experiments with Lentiviral shRNA-Lbp Particles

Mouse hepatoma Hepa1-6 cells were purchased from the American Type Culture Collection (ATCC, Virginia, EUA) and cultured in Dulbecco's Modified Eagle's Medium (DMEM) supplemented with 4500 mg/L glucose, 10% fetal bovine serum (Gibco), 100 units/ml penicillin and streptomycin, 1% glutamine and 1% sodium pyruvate, at 37° C. and 5% CO₂ atmosphere. Gene silencing was achieved using Lbp-targeted and control shRNA lentiviral particles. Stable clones expressing the shRNA were selected by puromycin dihydrochloride. Treatments were performed in Hepa1-6 cells for 24 h after seeding. Fatty acid accumulation was induced by palmitate exposure as follows: 27.84 mg of palmitate (Sigma, San Luis, MO) were dissolved in 1 ml sterile water to make a 100 mM stock solution. An aliquot of 5% bovine serum albumin (BSA) was prepared in serum-free DMEM. 100 mM palmitate stock solution and 5% BSA were mixed for at least 1 hour at 40° C. to obtain a 5 mM solution. Hepa1-6 cells were treated with 0.2 mM palmitate for 24 h. BSA supplemented medium was used as vehicle. Four biological replicates were collected from 2 independent experiments.

### Mitochondrial Respiratory Function

Mitochondrial respiratory function was assessed using Seahorse XFp Extracellular Flux Analyzer (Seahorse Bioscience, Agilent Technologies) using a Seahorse XFp Cell Mito Stress Test Kit according to the manufacturer's instructions. This assay determines basal respiration, ATP production, H⁺ (proton) leak, and spare respiratory capacity. Basal respiration shows energetic demand of the cell under baseline conditions. ATP production shows ATP produced by the mitochondria that contributes to meeting the energetic needs of the cell. Proton leak is the remaining basal respiration not coupled to ATP production and can be a sign of mitochondrial damage. Maximal respiration shows the maximum rate of respiration that the cell can achieve. Spare respiratory capacity indicates the capability of the cell to respond to an energetic demand and can be an indicator of cell fitness or flexibility.

### Western Blot Analysis

Liver (30 mg) and Hepa1-6 cells were homogenized in lysis cell buffer (50 mmol/l Tris-HCl pH 7.4, 150 mmol/l NaCl, 1.5 mmol/l MgCl₂, 1 mmol/l EDTA, 1 mmol/l EGTA, 40 mmol/l β-glycerophosphate, 2 mmol/l Na₃VO₄, 1 mmol/l PMSF, 1 mmol/l DTT) containing complete protease inhibitor cocktail (Roche Applied Science). Tissue and cell lysates were centrifuged at 1,500×g for 5 minutes at 4° C. to remove intact cells, and protein content was quantified using the Bradford method. For Western blotting, proteins (40 μg) were resolved by SDS-PAGE and transferred to a PVDF membrane (Immobilon; Millipore). Membranes were exposed overnight at 4° C. to primary antibodies anti-phospho-AMPKα (Thr172) (#2535) and anti-AMPKα (#2532) at 1/1000 dilution (Cell Signaling, Massachusetts, USA), and anti-β-actin at 1/1000 (sc-47778, Santa Cruz Biotechnology, Inc., Texas, USA) both diluted in 1×PBS containing 0.1% Tween-20, following the recommendations of the manufacturer. After secondary antibody incubation (Anti mouse/Rabbit IRP), signals were detected using enhanced chemiluminescence HRP substrate (Millipore) and analyzed with a luminescent image analyzer ChemiDoc MP Imaging System (BIO-RAD Laboratories, California, USA).

### Serum/Plasma Measurements

Human study. Plasma LBP was measured by human LBP enzyme-linked immunosorbent assay (ELISA) kit (HK315-02, HyCult Biotechnology, Huden, the Netherlands) with intra- and interassay coefficients of variation <8%. Serum glucose levels were measured in duplicate by the glucose oxidase method with a Beckman Glucose Analyzer 2 (Beckman Instruments, Brea, CA). The coefficient of variation (CV) was 1.9%. Total serum cholesterol was measured through the reaction of cholesterol esterase/oxidase/peroxidase, using a BM/Hitachi 747. HDL cholesterol was quantified after precipitation with polyethylene glycol at room temperature. Total serum triglycerides were measured through the reaction of glycerol-phosphate-oxidase and peroxidase by routine laboratory tests on a Hitachi 917 instrument (Roche, Mannheim, Germany). Glycosylated hemoglobin (HbA_{1c}) was measured by the high-performance liquid chromatography method (Bio-Rad, Muenchen, Germany, and autoanalyzer Jokoh HS-10, respectively). Intra- and interassay coefficients of variation were <4% for all these tests. C-reactive protein (ultrasensitive assay; 110 Beckman, Fullerton, CA, United States) was determined by a routine laboratory test.

Mouse experiments. Plasma LBP (HK205-02, LBP mouse ELISA kit, Hycult Biotech Inc., PA, USA), insulin (90080, Crystal Chem, Zaandam, Netherlands), glucose (Accutrend; Roche Diagnostics, Mannheim, Germany) and alanine transaminase (ALT) activity (Alfa Wasserman Vet Axcel Reagent SA1046) were measured using commercial kits according to manufacturer's instructions. For cytokine analysis in mouse serum samples the ProcartaPlex multiplex immunoassay (ThermoFisher) was used with a customized ProcataPlex panel [(Interleukin-6 (IL-6), Interleukin-10 (IL10), Interleukin 12p70 (IL-12p70), Interferon gamma (IFNg), Tumor Necrosis Factor (TNFa), and Monocyte chemoattractant protein 1 (MCP-1)). Samples were diluted and processed following the manufacturer's protocol. Standard curves for each cytokine were generated by using the reference cytokine concentrations supplied by the kit. Data was collected with a LuminexMAGPIX instrument system (Thermofisher) and analyzed with the Xponent (v4.2) software. Statistical analysis was done using Prism GraphPad software.

### Statistical Analysis

Statistical analyses were performed using the SPSS 12.0 software. For mouse experiments, all results are expressed as means±SEM, and differences were tested for statistical significance using Student's unpaired and paired t-tests, and non-parametric tests (Mann-Whitney U test). Levels of statistical significance were set at p<0.05.

### Example 1

This example describes the role of Lbp mRNA in the liver.

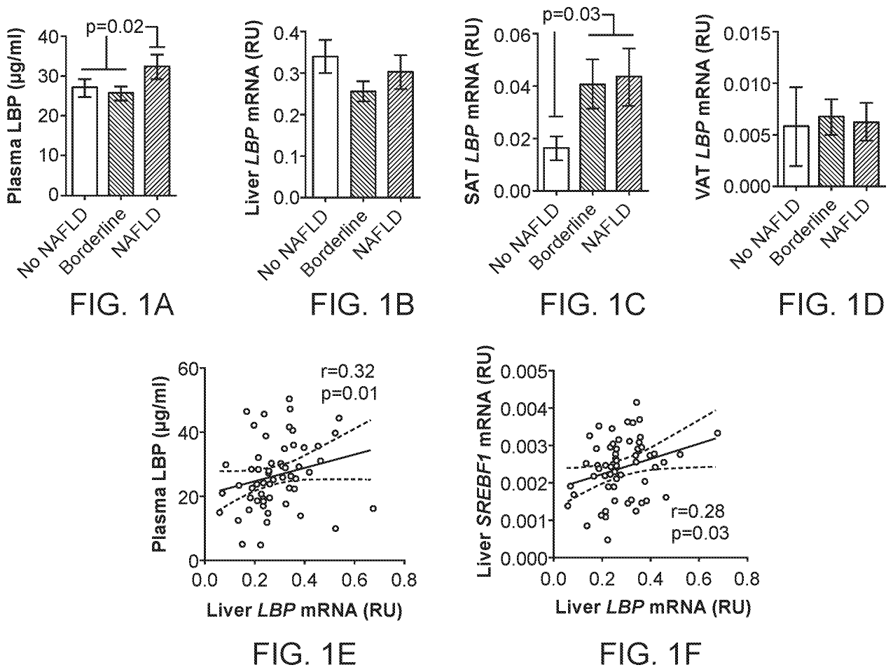

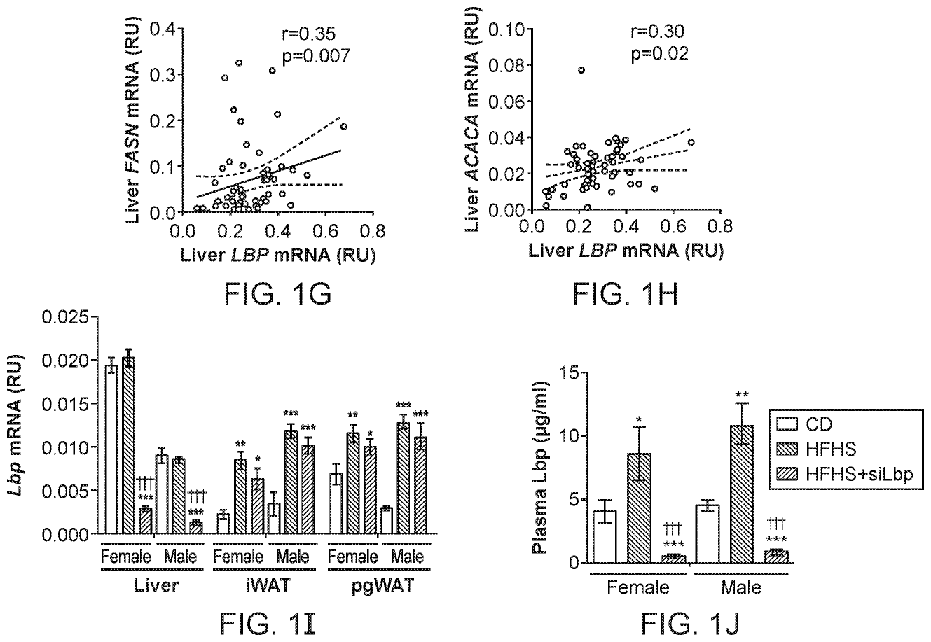

First, confirmation was obtained in morbidly obese subjects that circulating LBP was significantly increased in those subjects with hepatic steatosis evaluated through echography (FIG. 1A) (n=363, Table 1). Then, in a subgroup of morbidly obese participants (n=58, Table 2), in which hepatic pathology analysis was performed and liver, SAT and VAT RNA samples were available, LBP mRNA levels were analyzed. Again, circulating LBP was increased in morbidly obese patients with NAFLD (FIG. 1A). In contrast, liver LBP mRNA was not associated with NAFLD (FIG. 1). SAT, but not VAT, LBP mRNA levels were significantly increased in morbidly obese patients with hepatic steatosis (FIG. 1C-D), suggesting a possible contribution of SAT to hepatic steatosis-associated circulating LBP. However, LBP mRNA levels were significantly higher in liver than fat depots (p<0.0001 vs both SAT and VAT, respectively). In addition, circulating LBP concentrations were positively correlated with liver LBP (r=0.33, p=0.01, FIG. 1E), but not with SAT (r=0.09, p=0.6) or VAT (r=0.18, p=0.3) LBP mRNA levels. Liver LBP mRNA was positively correlated with expression of de novo lipogenesis-related genes (FASN, ACACA and SREBF1) (FIG. 1F-H).

These results show that circulating and liver-specific LBP protein levels, but not LBP mRNA, correlate with hepatic steatosis in humans. These results further show that liver Lbp mRNA is associated with de novo lipogenesis-related gene expression, but not hepatic steatosis in humans.

### Example 2

This example describes LNP-mediated delivery of Lbp siRNA.

Whether liver Lbp gene knockdown in high-fat high-sucrose-fed mice impacts plasma LBP levels and hepatic steatosis was tested next.

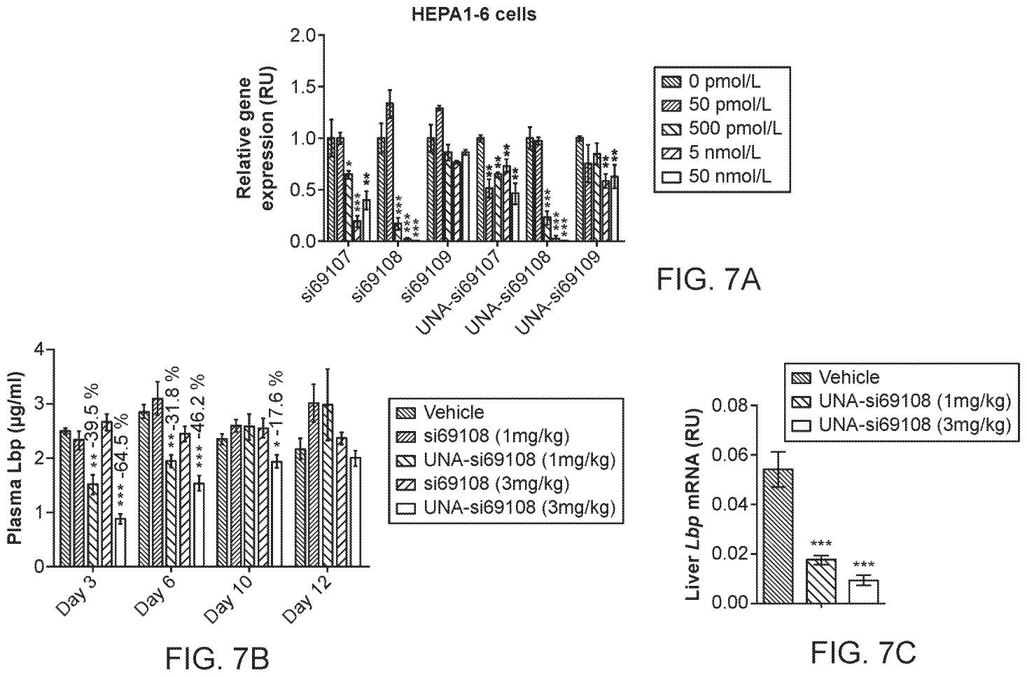

To test Lbp siRNA efficacy in vitro, three commercially available siRNAs (si69107, si69108, si69109) were screened at different doses in the Hepa1-6 cells. To increase siRNA stability and efficacy and to reduce the number of intravenous injections and potential immune responses in vivo, strategic introduction of chemical modifications in the three siRNAs was also tested. Both unmodified (si69108) and Unlocked nucleomonomer agent (UNA)-containing chemically modified (UNA-si69108) siRNA si69108 sequences resulted in the most significant Lbp gene knockdown (FIG. 7A) and were therefore selected to test in vivo, after having been formulated in lipid nanoparticles (LNPs; Arcturus Therapeutics). LNPs have been shown to deliver payloads specifically to mouse hepatocytes 18. Ten week-old male mice fed a high fat diet for two weeks were intravenously injected with either vehicle (phosphate buffer saline), LNPs with unmodified siRNA (LNP-si69108, 1 and 3 mg/kg) or LNPs with chemically modified siRNA (LNP-UNA-si69108, 1 and 3 mg/kg). Three days after injection, plasma LBP levels were significantly decreased in the groups treated with LNP-UNA-si69108 (FIG. 7B) and this effect was maintained until day 6. At day ten, serum LBP levels were significantly lower only at the highest dose, returning to baseline levels 12 days after injection (FIG. 7B). To confirm the dose-dependent inhibition of liver Lbp gene expression by LNP-UNA-si69108, a second intravenous injection was performed a week later in the same group of mice, at 1 and 3 mg/kg, and the mice were sacrificed after three days. Liver Lbp gene expression analysis confirmed the dose-dependent inhibition of liver Lbp mRNA levels by LNP-UNA-si69108 (FIG. 7C).

These results show that Lbp siRNA delivered through lipid nanoparticles reduced liver LBP gene expression and plasma LBP in vivo.

### Example 3

This example describes the effect of Lbp siRNA on high-fat-high-sucrose-induced liver lipid accumulation.

Whether treatment of mice with Lbp UNA-siRNA (UNA-si69108, from here on) could prevent lipid accumulation in the liver under obesogenic conditions was examined first. To set up this mouse model, mice were fed with a high fat and high sucrose diet (HFHS) known to promote liver steatosis²⁰ (FIG. 6A-C).

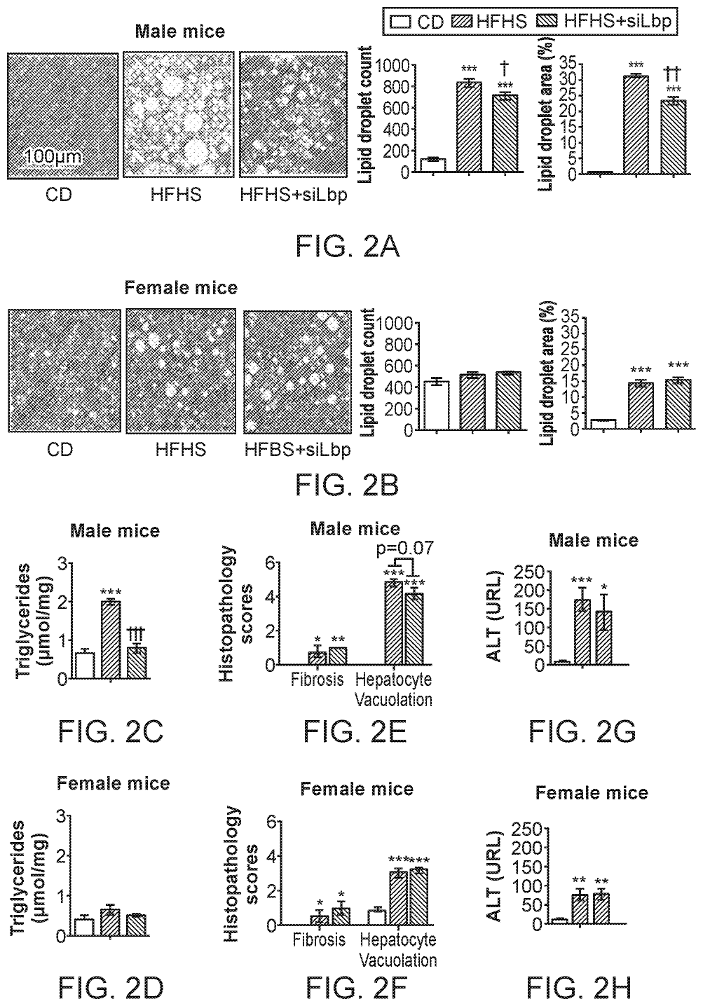

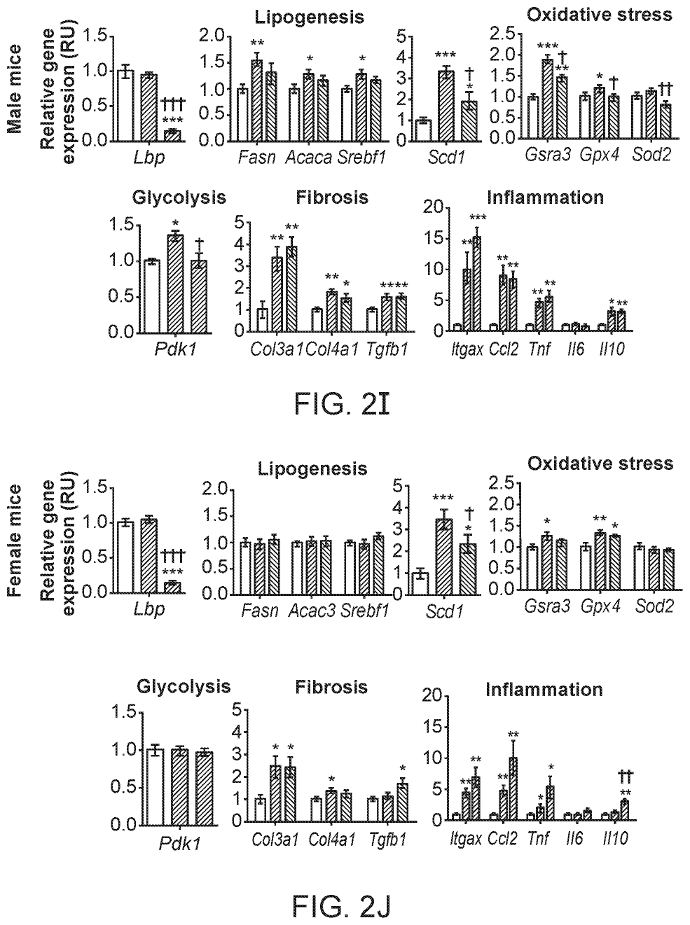

Diet effect. HFHS-induced obesity did not alter liver Lbp mRNA levels (FIG. 1I). Consistent with previous studies^{6,20,21}, both inguinal (iWAT) and perigonadal (pgWAT) white adipose tissue Lbp mRNA levels (FIG. 1I) and plasma LBP (FIG. 1J) were increased. HFHS resulted in a significantly increased liver lipid accumulation (FIG. 2A-F), including lipid droplet area (FIG. 2A-B), triglycerides (FIG. 2C-D), hepatocyte vacuolation, with a histopathology score ranging around 3 in females and between 4 and 5 in males and slightly but significantly increased fibrosis score (ranging between 0 and 1) in both males and females (FIG. 2E-F). Furthermore, increased serum ALT levels were also observed (FIG. 2G-H), supporting liver damage induced by HFHS. Importantly, some liver parameters showed sexual dimorphism. By contrast, compared to males, liver droplet count and area and hepatocyte vacuolation were higher in female mice fed a standard diet, but when fed HFHS, the increase in lipid accumulation and ALT levels was only observed in male mice (FIG. 2A-H). These data suggest that female mice are relatively protected from HFHS-induced liver lipid accumulation.

Treatment effect. HFHS-fed female and male mice were injected weekly with LNP-UNA-si69108 at the more effective dose (3 mg/kg) for the duration of the experiment (25 weeks), resulting in significantly decreased liver Lbp mRNA levels (85.7% in female and 84.6% in male mice compared with HFHS, p<0.0001) (FIG. 1I-J). Treated mice were compared with untreated under HFHS or control diet (CD).

In male mice, which were more affected than females by obesity-associated metabolic disturbances, LNP-Lbp UNA-si69108 administration decreased liver lipid droplet count and area and liver triglycerides, and tended to decreased hepatocyte vacuolation (FIG. 2A-F). Supporting these findings, increased expression of lipid metabolism (Fasn, Acaca, Scd1 and Srebf1)- and oxidative stress (Gsta3, Gpx4 and Sod2) genes in male HFHS-fed mice was significantly prevented by LNP-UNA-si69108 administration (FIG. 2I), indicating a role in the prevention of fatty liver disease. In contrast to male mice, and in line with histology analysis, HFHS did not increase the expression of lipid metabolism and oxidative-related genes (except for Scd1, Gsta3 and Gpx4) in females (FIG. 2J). Interestingly, in both males and females LNP-UNA-si69108 resulted in a significant decreased Scd1 gene expression compared to HFHS (FIG. 2I-J). Moreover, pyruvate dehydrogenase kinase 1 (PDK1), an inhibitor of pyruvate dehydrogenase activity, which promotes aerobic glycolysis²², was significantly increased in males, but not females after HFHS (FIG. 2I-J). Again, LNP-UNA-si69108 administration prevented the increase of Pdk1 gene expression (FIG. 2I).

In line with the slight, but significant, increase in fibrosis score in HFHS-fed mice, Col3a1 and Col4a1 gene expression levels were increased in both male and female mice while Tgfb1 only increased in males, all under HFHS (FIG. 2I-J). Inflammatory markers were also increased in both male and female HFHS-fed mice, including Itgax, Ccl2 and Tnf (FIG. 2I-J). Comparing untreated and treated with LNP-UNA-si69108 HFHS-fed mice, no significant differences between fibrosis and inflammatory markers (except for Il10 in female mice) were found (FIG. 2I-J). In addition, LNP-UNA-si69108 administration was not associated with a worsening in fibrosis and liver inflammation (FIG. 2E-H).

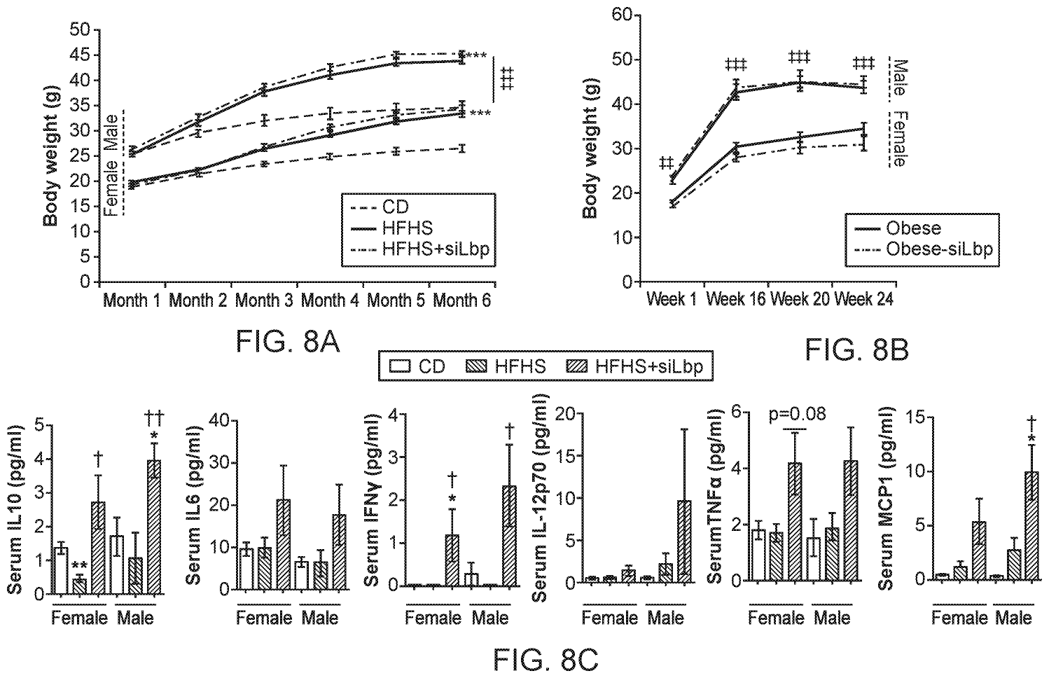

These data indicate that weekly LNP-UNA-si69108 treatment for 6 months is efficacious in reducing lipid accumulation, and safe with no additional liver damage or changes in body weight (FIG. 8A).

In summary, the above results show that Lbp siRNA delivered through lipid nanoparticles prevents high-fat-high-sucrose-induced liver lipid accumulation.

### Example 4

This example describes the effect of Lbp UNA-siRNA on liver steatosis in mice with established obesity.

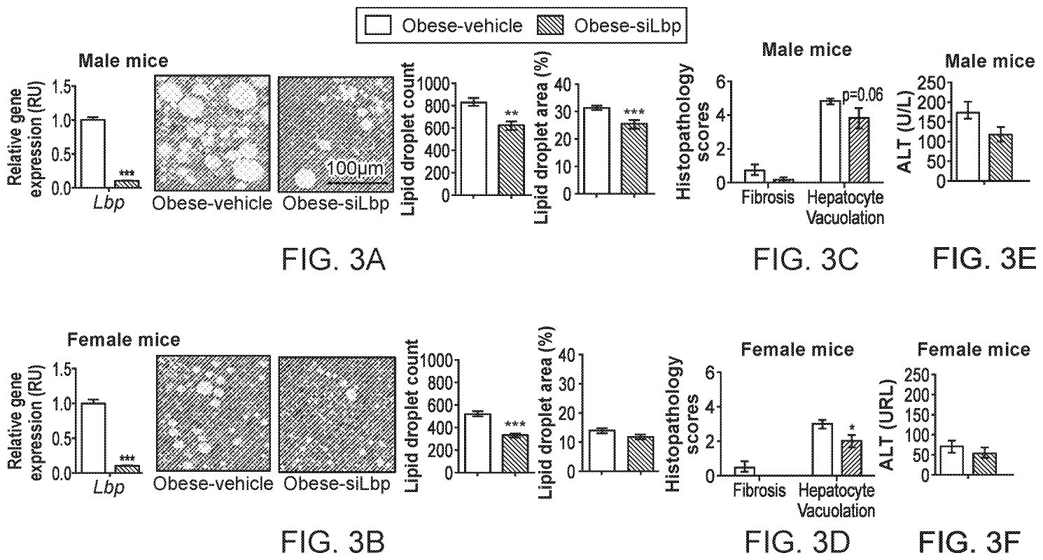

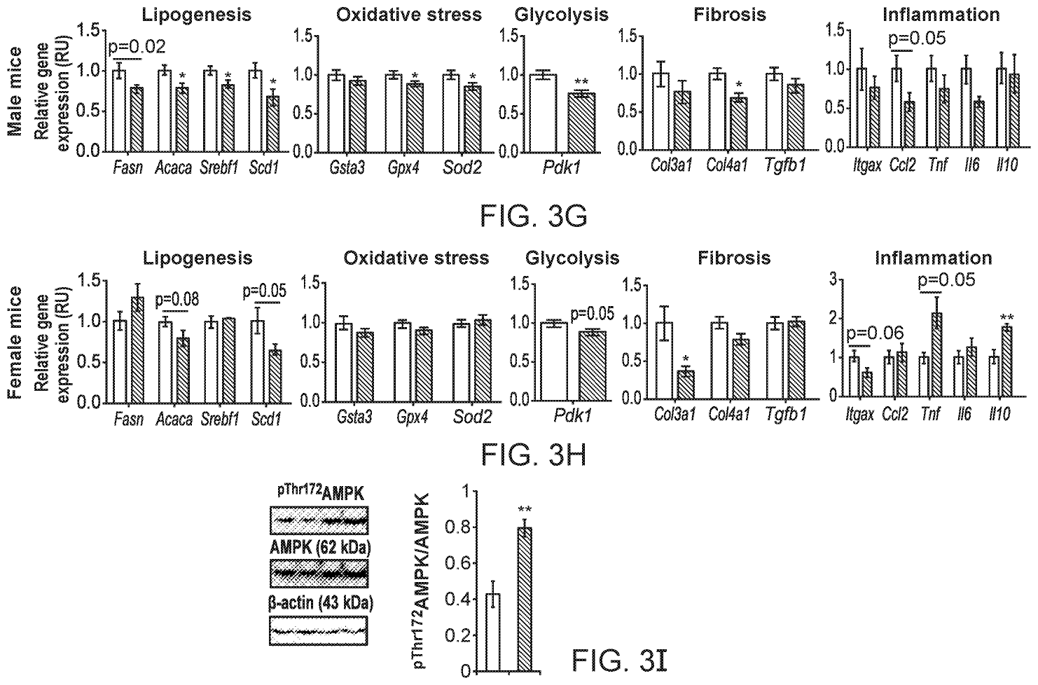

After observing that Lbp UNA-siRNA, UNA-si69108, delivered through lipid nanoparticles prevented lipid accumulation, whether this therapy could reverse established liver fat accumulation in mice with obesity was investigated next. Once mice reached a plateau of increased fat mass, weekly UNA-si69108 delivery through lipid nanoparticles was initiated and maintained for 8 weeks (FIG. 6D, FIG. 8B). This therapy led to reduced liver Lbp mRNA levels and in parallel decreased liver lipid accumulation (lipid droplet count and area and lipid vacuolation) both in males and females (FIG. 3A-D), with a non-significant decrease in fibrosis score and ALT levels (FIG. 3C-F). The intervention also resulted in decreased lipogenic and oxidative stress gene expression (Fasn, Acaca, Scd1, Srefb1, Gpx4 and Sod2 in males and Acaca and Scd1 in females) and in reduced Pdk1 mRNA levels in both males and females (FIG. 3G-H). Of note, Lbp UNA-siRNA administration led to increased liver^{pThr172} AMPK phosphorylation (FIG. 3I). Notably, the expression of some fibrosis and inflammation-related genes (Col4a1, Ccl2 and Il6 mRNA levels in males and Col3a1 in females) decreased after treatment. The anti-inflammatory cytokine Il-10 significantly increased in females (FIG. 3G-H).

This second experiment demonstrated that Lbp UNA-siRNA administration in obese mice also resulted in liver fat reduction.

In summary, these results show that Lbp UNA-siRNA delivered through lipid nanoparticles improved liver steatosis in mice with established obesity.

### Example 5

This example describes tolerability of Lbp UNA-siRNA delivered through lipid nanoparticles.

To evaluate the effects of weekly LNP-UNA-si69108 administration, inflammatory parameters that are part of the response to lipid nanoparticle treatment were evaluated. Several cytokines were measured in serum by Luminex assay, including IL10, IL6, IFNγ, IL12p70, TNFα and MCP1, after 25 weeks treatment in CD and HFHS (FIG. 8C). Compared to CD, no significant effects of HFHS on serum levels of these cytokines were observed in both males and females, except for a significant decrease in serum IL-10 levels in females (FIG. 8C). In both male and female mice, LNP-UNA-si69108 administration increased the anti-inflammatory cytokine IL-10, and only in males a slight increase in MCP1 levels was observed (FIG. 8C). Taken together, the cytokine profiles suggest that long-term administration of UNA-siRNA using lipid nanoparticles was safe even in obesity mouse models.

These results show that long-term therapy with Lbp UNA-siRNA delivered through lipid nanoparticles was well tolerated.

### Example 6

This example describes the effect of lipid formulated-LBP siRNA administration on insulin action.

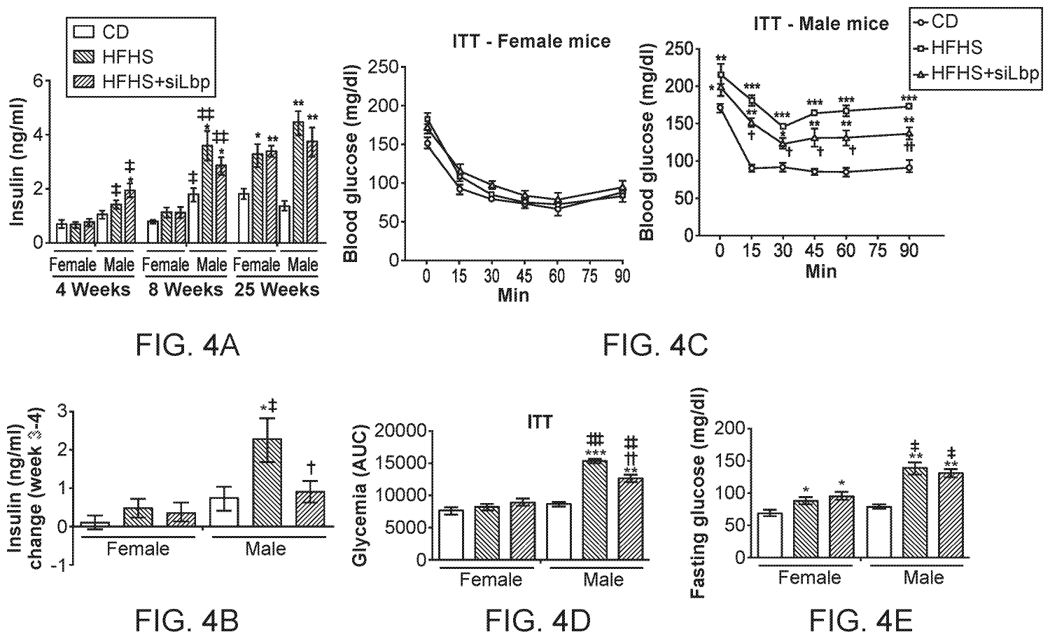

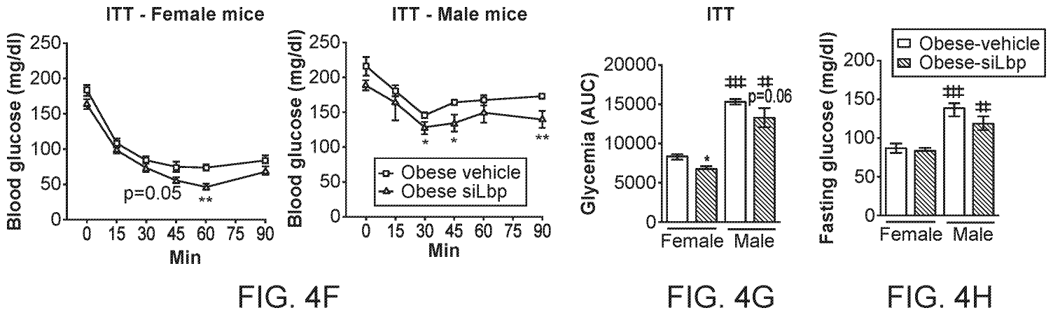

In an obesity progression experiment, time-course analysis of insulin levels at weeks 4 and 8 showed gender differences, with increased insulin levels in male compared to female mice (FIG. 4A) and increased insulin levels in HFHS compared to CD at week 8 only in male mice (FIG. 4A). Of note, LNP-UNA-si69108, treatment significantly reduced the rate of insulin increase from week 4 to 8 in male mice (FIG. 4B). To investigate if LNP-LBP siRNA administration exerted positive effects on insulin sensitivity, an insulin tolerance test (ITT) was performed at week 20. While female mice did not show HFHS-induced insulin resistance (FIG. 4C), increased insulin resistance in HFHS-fed male mice was significantly improved with liver and plasma LBP depletion (HFHS+siLBP) (FIG. 4C-F). Similar to ITT, and in line with the healthier metabolic phenotype observed in liver parameters in females (2A-H), normal fasting glucose levels in HFHS-fed female mice (FIG. 4E) were found. Other than that, comparing HFHS+L and HFHS groups, there were no significant differences in fasting insulin and glucose levels at week 25 (FIG. 4A and FIG. 4E). Taking together these data show that females are more resistant to metabolic changes induced by HFHS, and LNP-LBP siRNA is able to improve insulin action in male mice with increased insulin resistance.

Strengthening these findings, diet-induced obese mice treated with LNP-LBP siRNA for 8 weeks also showed reduced glycemia at 30, 45 and 90 min in males and at 60 min in females and improved glycemia AUC during ITT (FIG. 4F-G), without significant effects on fasting glucose (FIG. 4H). Similar to previous experiments, when male and female mice were compared, impaired ITT and increased fasting glucose in male mice were found (FIG. 4F-H).

These results show that lipid formulated-LBP siRNA administration improved insulin action in high-fat-high-sucrose fed mice.

### Example 7

This example describes the effect of Lbp gene knockdown on mitochondrial respiratory capacity.

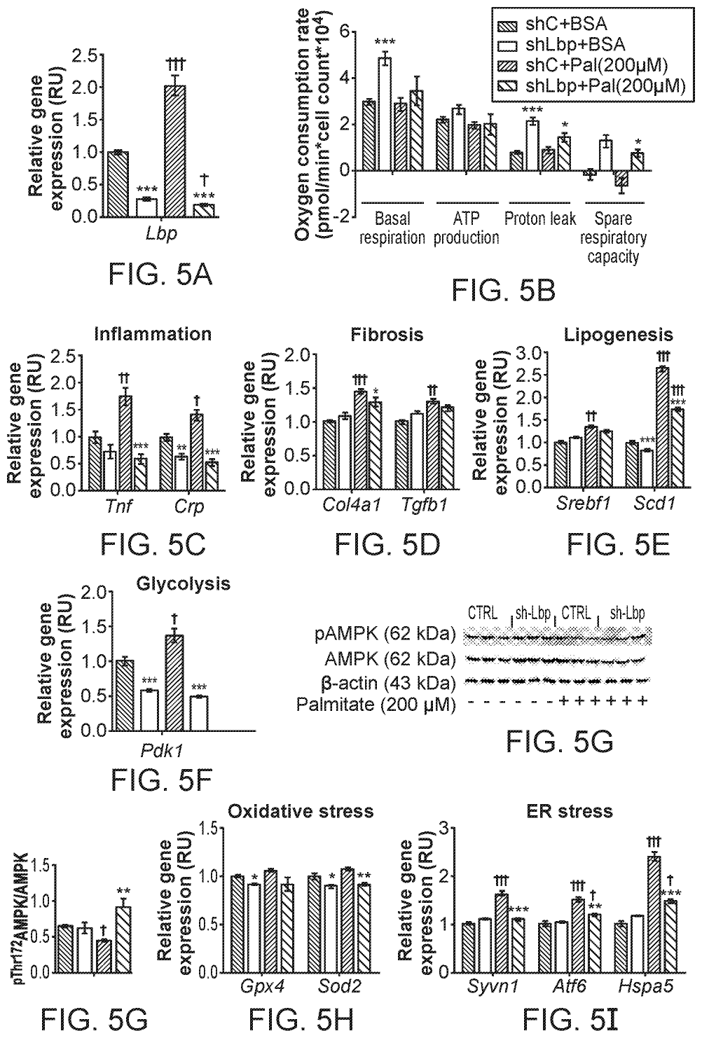

To gain insight in the potential mechanisms that underlie the positive effects of Lbp depletion on liver metabolism, in vitro experiments were performed in the murine Hepa1-6 cell line. Lbp gene knockdown (KD) using lentiviral particles with Lbp-specific shRNA (FIG. 5A) resulted in significant improvements in mitochondrial respiratory capacity (FIG. 5B), enhancing basal respiration, and uncoupled oxygen consumption (proton leak) and spare respiratory activity, even after palmitate treatment, but without altering its efficiency in ATP production (FIG. 5B).

These results show that Lbp gene knockdown improved mitochondrial respiratory capacity.

### Example 8

This example describes the effect of Lbp gene knockdown (KD) on palmitate.

Palmitate (200 μM, 24 h) administration in mouse Hepa1-6 cells resulted in increased expression of Lbp in parallel to inflammatory (Tnf Crp), fibrosis (Col4a1, Tgfb1), and metabolic (Srebf1, Scd1 and Pdk1)-related gene expression (FIG. 5C-F). Under these conditions of palmitate excess, Lbp gene KD resulted in decreased Tnf Crp, Col4a1, Scd1 and Pdk1 and prevented the increase in Tgfb1 and Srebf1 gene expression (FIG. 5C-F) and inhibition of AMPK activity, as shown by the increased ratio^{pThr172} AMPK/AMPK (FIG. 5G). Interestingly, under both normal and palmitate excess conditions, Lbp gene KD attenuated expression of oxidative stress markers (Gpx4 and Sod2) (FIG. 5H) and endoplasmic reticulum stress markers (Syvn1, Atf6 and Hspa5) upon induction by palmitate (FIG. 5I).

These results show that Lbp gene knockdown attenuated the detrimental effects of palmitate in vitro.

### Discussion of Examples 1-8

Metabolic-associated fatty liver disease (MAFLD) is one of the main consequences of the overall burden of obesity worldwide and contributes to aggravate other obesity-associated metabolic disturbances, such as insulin resistance, dyslipidemia and type 2 diabetes, sharing a common pathophysiology¹⁻⁴. Circulating lipopolysaccharide-binding protein (LBP) has been identified as a relevant component of innate immunity linked to obesity, insulin resistance and the metabolic syndrome⁵⁻⁹. Circulating LBP has also been described to be associated with liver fat accumulation and fibrosis in humans¹⁰⁻¹³. The liver is the main source of circulating LBP, although it is also present in the adipose tissue¹⁵⁻¹⁷.

To the best of available knowledge, the impact of Lbp on diet-induced liver steatosis has only been evaluated in one previous study¹⁴ that was based on a LBP KO mouse model. The current study was designed to specifically inhibit Lbp in liver in wild-type mice. Thus, this is the first study testing the therapeutic potential of liver Lbp for liver steatosis. Unlike studies that examined the protective effects of Lbp inhibition under conditions of liver injury²³⁻²⁶, the current study focused, for the first time, on the early stages of obesity-associated liver steatosis. The delivery of lipid nanoparticles containing Lbp UNA-siRNA had a preventive effect on liver steatosis after a high fat diet in mice with a prediabetes-like phenotype. Without being limited by theory, several points of evidence suggest that these beneficial effects of the treatment are mainly due to liver-specific LBP reduction.

Lbp gene knockdown by Lbp UNA-siRNA decreased expression of important genes in hepatocyte lipid metabolism, like Stearoyl-coenzyme A desaturase (Scd1), a gene involved in triglyceride biosynthesis and upregulated in liver steatosis²⁷. Without being limited by theory, the consistently attenuated Scd1 mRNA levels in all experiments suggest that part of the beneficial effects of the treatment could be through Scd1 suppression. Targeting of Scd1 has been shown to prevent and attenuate NASH progression through the inhibition of liver de novo lipogenesis and increased oxidative phosphorylation activity²⁸⁻³⁰.

Reduction of oxidative phosphorylation activity is a cellular adaptation/response to a high fat diet³¹. Since Pdk1 inhibits pyruvate dehydrogenase activity, enhancing aerobic glycolysis²², the downregulation of Pdk1 mRNA levels after liver Lbp KD could improve mitochondrial dysfunction and free fatty acid β-oxidation, and consequently prevent liver lipid accumulation and oxidative stress³²⁻³⁴. Considering that disturbances in lipid metabolism^{35,36} and oxidative stress^{32,33} play an important role in MAFLD pathogenesis, studies described herein point to a possible role of LBP in early stages of MAFLD progression, and not only in NASH-associated fibrosis and inflammation as previously reported¹⁴. In line with this, studies described herein showed that Lbp KD resulted in a significant reduction in oxidative stress markers. In addition, increased mitochondrial respiration rate detected in Lbp KD cells was due to increased proton leak (uncoupled respiration), an important mitochondrial process in the prevention of oxidative stress^{37,38}. A role of mitochondrial function in liver steatosis has been shown³⁹⁻⁴¹. Furthermore, Lbp KD in mouse liver and Hepa1-6 cells enhanced activity of the 5′ AMP-activated protein kinase (AMPK), an important enzyme promoting fat metabolism and mitochondrial respiration and inhibiting de novo lipogenesis⁴²⁻⁴⁴.

Mechanistically, it is important to note that the positive charges of LBP that allow its interaction with LPS also confer to LBP the capacity to bind to negatively charged lipids like phosphatidylinositol or phosphatidylserine⁴⁵, allowing its intercalation into phospholipid membranes⁴⁶. In addition, previous studies suggest that LBP acts as a lipid exchange protein, catalyzing an equilibration of amphiphilic, membrane-forming lipids between membranes^{47,48}. These studies also suggest a role of LBP in the modulation of intracellular phospholipids. Supporting this suggestion, adipose tissue LBP was negatively associated with some phospholipid species (including phosphatidylcholines and phosphatidylserines) and in adipocytes, Lbp gene KD led to sharp changes in the lipidome, reducing lipid species linked to inflammation and insulin resistance in parallel with improved mitochondrial respiratory capacity¹⁵. Of interest, phosphatidylserine transfer to mitochondria is required to prevent the progression of fatty liver disease⁴⁹.

A challenge for using LNPs as a therapeutic delivery system is the potential induction of liver damage and undesired stimulation of immune responses⁵⁰. However, the chemical properties of the LNP system used in the present studies have been shown to make it a successful, safe and potent siRNA delivery system¹⁸. The studies described herein demonstrated that long term treatment (6 months) with repeated weekly doses of LNP-Lbp UNA-siRNA was well tolerated by the mouse liver and did not have an impact on serum ALT levels, liver fibrosis score, and liver fibrosis- and inflammation-related gene expression, supporting the safety profile of LNPs used herein. The cytokine profile of mice treated with LNP-Lbp UNA-siRNA for 6 months was also found not to be adverse. A slight but significant increase in IL-10 levels in both males and females, and MCP-1 levels in males, was detected. Without being limited by theory, even though LNP-Lbp UNA-siRNA administration might stimulate tissue macrophage recruitment through the chemokine MCP-1, these cells would polarize into an IL-10-associated anti-inflammatory phenotype, which might explain in part the positive effects of this intervention attenuating liver steatosis⁵²⁻⁵⁴.

Since liver lipid accumulation is considered an important contributor to obesity-associated hepatic insulin resistance⁴, the potential therapeutic effects of LNP-Lbp UNA-siRNA might be extended to improve insulin action. LNP-UNA siRNA LBP also resulted in a significant decrease of glycemia in insulin tolerance tests, indicating positive effects improving insulin action, mainly in male mice. In agreement with these findings, a recent study demonstrated that the inhibition of circulating and liver LBP in non-obese mice fed with standard diet chow resulted in improved glucose levels and glucose tolerance⁵. The effects of a high fat diet or in obese mice were not evaluated in this latter study.

Interestingly, liver-specific LBP knockdown by LNP-LBP siRNA intervention revealed different effects on metabolism according to sex. Without being limited by theory, sex differences could be due to protective effects of sexual hormones, specifically estrogen, on obesity-associated metabolic disturbances, including liver steatosis³³ and insulin resistance⁵⁶. At the same age and diet (high sucrose and high fat diet), female mice displayed decreased fasting glucose and insulin levels and improved insulin and glucose tolerance. However, male mice were not resistant to HFHS-associated metabolic disturbances, showing increased insulin resistance (increased AUC ITT at week 20 and hyperinsulinemia at week 8). LNP-LBP siRNA administration resulted in significant metabolic benefits, reducing hyperinsulinemia, improving insulin action and decreasing liver lipid accumulation in mice with a prediabetes-like phenotype (males), but not in those with a metabolic healthier phenotype (females).

Another important finding of the current study was that the increase in plasma LBP linked to obesity and liver steatosis (reported in current and previous studies⁵⁻¹³) was associated with adipose tissue, but not liver Lbp gene expression. Without being limited by theory, these data indicate that even though the liver is the main source of plasma Lbp concentration, because specific liver Lbp gene knockdown produced a significant reduction (85%, p<0.0001) of plasma Lbp, fat depots could be a relevant contributor to obesity-associated increased plasma Lbp levels. Studies designed to evaluate the specific role of adipose tissue Lbp biosynthesis in obesity-associated metabolic disturbances are described below (Examples 9-13).

In summary, taken together, results described herein substantiated the importance of liver Lbp gene knockdown through lipid nanoparticles in the prevention and therapy of obesity-associated liver steatosis, constituting a potential target for MAFLD therapy.

### References for Examples 1-8

[1] Gluchowski N L, Becuwe M, Walther T C, Farese R V. Lipid droplets and liver disease: From basic biology to clinical implications. Nat Rev Gastroenterol Hepatol 2017; 14:343-55. https://doi.org/10.1038/nrgastro.2017.32.
[2] Taylor R, Al-Mrabeh A, Sattar N. Understanding the mechanisms of reversal of type 2 diabetes. Lancet Diabetes Endocrinol 2019; 7:726-36. https://doi.org/10.1016/S2213-8587(19)30076-2.
[3] Heymsfield S B, Wadden T A. Mechanisms, Pathophysiology, and Management of Obesity. N Engl J Med 2017; 376:254-66. https://doi.org/10.1056/NEJMra1514009.
[4] Al-Mrabeh A, Zhyzhneuskaya S V., Peters C, Barnes A C, Melhem S, Jesuthasan A, et al. Hepatic Lipoprotein Export and Remission of Human Type 2 Diabetes after Weight Loss. Cell Metab 2020; 31:233-249.e4. https://doi.org/10.1016/j.cmet.2019.11.018.
[5] Sun L, Yu Z, Ye X, Zou S, Li H, Yu D, et al. A Marker of Endotoxemia Is Associated With Obesity and Related Metabolic Disorders in Apparently Healthy Chinese. Diabetes Care 2010; 33:1925-32. https://doi.org/10.2337/dc10-0340.
[6] Moreno-Navarrete J M, Ortega F, Serino M, Luche E, Waget A, Pardo G, et al. Circulating lipopolysaccharide-binding protein (LBP) as a marker of obesity-related insulin resistance. Int J Obes 2012; 36:1442-9. https://doi.org/10.1038/ijo.2011.256.
[7] Kheirandish-Gozal L, Peris E, Wang Y, Kakazu M T, Khalyfa A, Carreras A, et al. Lipopolysaccharide-Binding protein plasma levels in children: Effects of obstructive sleep apnea and obesity. J Clin Endocrinol Metab 2014; 99:656-63. https://doi.org/10.1210/jc.2013-3327.
[8] Liu X, Lu L, Yao P, Ma Y, Wang F, Jin Q, et al. Lipopolysaccharide binding protein, obesity status and incidence of metabolic syndrome: A prospective study among middle-aged and older Chinese. Diabetologia 2014; 57:1834-41. https://doi.org/10.1007/s00125-014-3288-7.
[9] Tilves C M, Zmuda J M, Kuipers A L, Nestlerode C S, Evans R W, Bunker C H, et al. Association of lipopolysaccharide-binding protein with aging-related adiposity change and prediabetes among African ancestry men. Diabetes Care 2016; 39:385-91. https://doi.org/10.2337/dc15-1777.
[10] Nien H C, Sheu J C, Chi Y C, Chen C L, Kao J H, Yang W S. One-year weight management lowers lipopolysaccharide-binding protein and its implication in metainflammation and liver fibrosis. PLoS One 2018; 13:e0207882-e0207882. https://doi.org/10.1371/journal.pone.0207882.
[11] Pang J, Xu W, Zhang X, Wong G L H, Chan A W H, Chan H Y, et al. Significant positive association of endotoxemia with histological severity in 237 patients with non-alcoholic fatty liver disease. Aliment Pharmacol Ther 2017; 46:175-82. https://doi.org/10.1111/apt.14119.
[12] Kitabatake H, Tanaka N, Fujimori N, Komatsu M, Okubo A, Kakegawa K, et al. Association between endotoxemia and histological features of nonalcoholic fatty liver disease. World J Gastroenterol 2017; 23:712-22. https://doi.org/10.3748/wjg.v23.i4.712.
[13] Nier A, Engstler A J, Maier I B, Bergheim I. Markers of intestinal permeability are already altered in early stages of non-alcoholic fatty liver disease: Studies in children. PLoS One 2017; 12. https://doi.org/10.1371/journal.pone.0183282.
[14] Jin C J, Engstler A J, Ziegenhardt D, Bischoff S C, Trautwein C, Bergheim I. Loss of lipopolysaccharide-binding protein attenuates the development of diet-induced non-alcoholic fatty liver disease in mice. J Gastroenterol Hepatol 2017; 32:708-15. https://doi.org/10.1111/jgh.13488.
[15] Moreno-Navarrete J M, Jove M, Padró T, Boada J, Ortega F, Ricart W, et al. Adipocyte lipopolysaccharide binding protein (LBP) is linked to a specific lipidomic signature. Obesity 2017; 25:391-400. https://doi.org/10.1002/oby.21711.
[16] Martin T R, Mathison J C, Tobias P S, Leturcq D J, Moriarty A M, Maunder R J, et al. Lipopolysaccharide binding protein enhances the responsiveness of alveolar macrophages to bacterial lipopolysaccharide. Implications for cytokine production in normal and injured lungs. J Clin Invest 1992; 90:2209-19. https://doi.org/10.1172/JCI116106.
[17] Moreno-Navarrete J M, Escoté X, Ortega F, Serino M, Campbell M, Michalski M C, et al. A role for adipocyte-derived lipopolysaccharide-binding protein in inflammation- and obesity-associated adipose tissue dysfunction. Diabetologia 2013; 56:2524-37. https://doi.org/10.1007/s00125-013-3015-9.
[18] Ramaswamy S, Tonnu N, Tachikawa K, Limphong P, Vega J B, Karmali P P, et al. Systemic delivery of factor IX messenger RNA for protein replacement therapy. Proc Natl Acad Sci USA 2017; 114:E1941-50. https://doi.org/10.1073/pnas.1619653114.
[19] Seco C Z, Castells-Nobau A, Joo S H, Schraders M, Foo J N, Van Der Voet M, et al. A homozygous FITM2 mutation causes a deafness-dystonia syndrome with motor regression and signs of ichthyosis and sensory neuropathy. DMM Dis Model Mech 2017; 10:105-18. https://doi.org/10.1242/dmm.026476.
[20] Stein S, Lemos V, Xu P, Demagny H, Wang X, Ryu D, et al. Impaired SUMOylation of nuclear receptor LRH-1 promotes nonalcoholic fatty liver disease. J Clin Invest 2017; 127:583-92. https://doi.org/10.1172/JCI85499.
[21] Dewhurst-Trigg R, Wadley A J, Woods R M, Sherar L B, Bishop N C, Hulston C J, et al. Short-term High-fat Overfeeding Does Not Induce NF-κB Inflammatory Signaling in Subcutaneous White Adipose Tissue. J Clin Endocrinol Metab 2020; 105. https://doi.org/10.1210/clinem/dgaa158.
[22] Hong S M, Lee Y K, Park I, Kwon S M, Min S, Yoon G. Lactic acidosis caused by repressed lactate dehydrogenase subunit B expression down-regulates mitochondrial oxidative phosphorylation via the pyruvate dehydrogenase (PDH)-PDH kinase axis. J Biol Chem 2019; 294:7810-20. https://doi.org/10.1074/jbc.RA118.006095.
[23] Su G L, Gong K Q, Fan M H, Kelley W M, Hsieh J, Sun J M, et al. Lipopolysaccharide-binding protein modulates acetaminophen-induced liver injury in mice. Hepatology 2005; 41:187-95. https://doi.org/10.1002/hep.20533.
[24] Uesugi T, Froh M, Arteel G E, Bradford B U, Wheeler M D, Gabele E, et al. Role of Lipopolysaccharide-Binding Protein in Early Alcohol-Induced Liver Injury in Mice. J Immunol 2002; 168:2963-9. https://doi.org/10.4049/jimmunol.168.6.2963.
[25] Su G L, Hoesel L M, Bayliss J, Hemmila M R, Wang S C. Lipopolysaccharide binding protein inhibitory peptide protects against acetaminophen-induced hepatotoxicity. Am J Physiol Liver Physiol 2010; 299:G1319-25. https://doi.org/10.1152/ajpgi.00140.2010.
[26] Minter R M, Bi X, Ben-Josef G, Wang T, Hu B, Arbabi S, et al. LPS-binding protein mediates LPS-induced liver injury and mortality in the setting of biliary obstruction. Am J Physiol—Gastrointest Liver Physiol 2009; 296:G45-54. https://doi.org/10.1152/ajpgi.00041.2008.
[27] Benhammou J N, Ko A, Alvarez M, Kaikkonen M U, Rankin C, Garske K M, et al. Novel Lipid Long Intervening Noncoding RNA, Oligodendrocyte Maturation-Associated Long Intergenic Noncoding RNA, Regulates the Liver Steatosis Gene Stearoyl-Coenzyme A Desaturase As an Enhancer RNA. Hepatol Commun 2019; 3:1356-72. https://doi.org/10.1002/hep4.1413.
[28] Ntambi J M, Miyazaki M, Stoehr J P, Lan H, Kendziorski C M, Yandell B S, et al. Loss of stearoyl-CoA desaturase-1 function protects mice against adiposity. Proc Natl Acad Sci USA 2002; 99:11482-6. https://doi.org/10.1073/pnas.132384699.
[29] Iruarrizaga-Lejarreta M, Varela-Rey M, Fernindez-Ramos D, Martinez-Arranz I, Delgado T C, Simon J, et al. Role of aramchol in steatohepatitis and fibrosis in mice. Hepatol Commun 2017; 1:911-27. https://doi.org/10.1002/hep4.1107.
[30] Safadi R, Konikoff F M, Mahamid M, Zelber-Sagi S, Halpern M, Gilat T, et al. The Fatty Acid-Bile Acid Conjugate Aramchol Reduces Liver Fat Content in Patients With Nonalcoholic Fatty Liver Disease. Clin Gastroenterol Hepatol 2014; 12:2085-2091.el. https://doi.org/10.1016/j.cgh.2014.04.038.
[31] Kahle M, Schafer A, Seelig A, Schultheilß J, Wu M, Aichler M, et al. High fat diet-induced modifications in membrane lipid and mitochondrial-membrane protein signatures precede the development of hepatic insulin resistance in mice. Mol Metab 2015; 4:39-50. https://doi.org/10.1016/j.molmet.2014.11.004.
[32] Bessone F, Razori M V, Roma M G. Molecular pathways of nonalcoholic fatty liver disease development and progression. Cell Mol Life Sci 2019; 76:99-128. https://doi.org/10.1007/s00018-018-2947-0.
[33] Galmes-Pascual B M, Martinez-Cignoni M R, Morin-Costoya A, Bauza-Thorbrugge M, Sbert-Roig M, Valle A, et al. 17β-estradiol ameliorates lipotoxicity-induced hepatic mitochondrial oxidative stress and insulin resistance. Free Radic Biol Med 2020; 150:148-60. https://doi.org/10.1016/j.freeradbiomed.2020.02.016.
[34] Aoudjehane L, Gautheron J, Le Goff W, Goumard C, Gilaizeau J, Nget C S, et al. Novel defatting strategies reduce lipid accumulation in primary human culture models of liver steatosis. DMM Dis Model Mech 2020; 13. https://doi.org/10.1242/dmm.042663.
[35] Min H K, Kapoor A, Fuchs M, Mirshahi F, Zhou H, Maher J, et al. Increased hepatic synthesis and dysregulation of cholesterol metabolism is associated with the severity of nonalcoholic fatty liver disease. Cell Metab 2012; 15:665-74. https://doi.org/10.1016/j.cmet.2012.04.004.
[36] Chiappini F, Coilly A, Kadar H, Gual P, Tran A, Desterke C, et al. Metabolism dysregulation induces a specific lipid signature of nonalcoholic steatohepatitis in patients. Sci Rep 2017; 7. https://doi.org/10.1038/srep46658.
[37] Cadenas S. Mitochondrial uncoupling, ROS generation and cardioprotection. Biochim Biophys Acta—Bioenerg 2018; 1859:940-50. https://doi.org/10.1016/j.bbabio.2018.05.019.
[38] Brookes P S. Mitochondrial H+leak and ROS generation: An odd couple. Free Radic Biol Med 2005; 38:12-23. https://doi.org/10.1016/j.freeradbiomed.2004.10.016.
[39] Barbier-Torres L, Fortner K A, Iruzubieta P, Delgado T C, Giddings E, Chen Y, et al. Silencing hepatic MCJ attenuates non-alcoholic fatty liver disease (NAFLD) by increasing mitochondrial fatty acid oxidation. Nat Commun 2020; 11. https://doi.org/10.1038/s41467-020-16991-2.
[40] Dornas W, Schuppan D. Mitochondrial oxidative injury: A key player in nonalcoholic fatty liver disease. Am J Physiol Liver Physiol 2020; 319. https://doi.org/10.1152/ajpgi.00121.2020.
[41] Meex R C R, Blaak E E. Mitochondrial Dysfunction is a Key Pathway that Links Saturated Fat Intake to the Development and Progression of NAFLD. Mol Nutr Food Res 2020. https://doi.org/10.1002/mnfr.201900942.
[42] Fondevila M F, Fernandez U, Gonzalez-Rellan M J, Da Silva Lima N, Buque X, Gonzalez-Rodriguez A, et al. The L-α-lysophosphatidylinositol/GPR55 system induces the development of non-alcoholic steatosis and steatohepatitis. Hepatology 2020. https://doi.org/10.1002/hep.31290.
[43] Kim M H, Seong J B, Huh J W, Bae Y C, Lee H S, Lee D S. Peroxiredoxin 5 ameliorates obesity-induced non-alcoholic fatty liver disease through the regulation of oxidative stress and AMP-activated protein kinase signaling. Redox Biol 2020; 28. https://doi.org/10.1016/j.redox.2019.101315.
[44] Han Y, Hu Z, Cui A, Liu Z, Ma F, Xue Y, et al. Post-translational regulation of lipogenesis via AMPK-dependent phosphorylation of insulin-induced gene. Nat Commun 2019; 10. https://doi.org/10.1038/s41467-019-08585-4.
[45] Mueller M, Brandenburg K, Dedrick R, Schromm A B, Seydel U. Phospholipids Inhibit Lipopolysaccharide (LPS)-Induced Cell Activation: A Role for LPS-Binding Protein. J Immunol 2005; 174:1091-6. https://doi.org/10.4049/jimmunol.174.2.1091.
[46] Roes S, Mumm F, Seydel U, Gutsmann T. Localization of the lipopolysaccharide-binding protein in phospholipid membranes by atomic force microscopy. J Biol Chem 2006; 281:2757-63. https://doi.org/10.1074/jbc.M507634200.
[47] Yu B, Hailman E, Wright S D. Lipopolysaccharide binding protein and soluble Cd14 catalyze exchange of phospholipids. J Clin Invest 1997; 99:315-24. https://doi.org/10.1172/JCI119160.
[48] Schromm A B, Brandenburg K, Rietschel E T, Flad H D, Carroll S F, Seydel U. Lipopolysaccharide-binding protein mediates CD14-independent intercalation of lipopolysaccharide into phospholipid membranes. FEBS Lett 1996; 399:267-71. https://doi.org/10.1016/S0014-5793(96)01338-5.
[49] Hernández-Alvarez M I, Sebastán D, Vives S, Ivanova S, Bartoccioni P, Kakimoto P, et al. Deficient Endoplasmic Reticulum-Mitochondrial Phosphatidylserine Transfer Causes Liver Disease. Cell 2019; 177:881-895.e17. https://doi.org/10.1016/j.cell.2019.04.010.
[50] Cullis P R, Hope M J. Lipid Nanoparticle Systems for Enabling Gene Therapies. Mol Ther 2017; 25:1467-75. https://doi.org/10.1016/j.ymthe.2017.03.013.
[51] Adams D, Gonzalez-Duarte A, O'Riordan W D, Yang C C, Ueda M, Kristen A V., et al. Patisiran, an RNAi therapeutic, for hereditary transthyretin amyloidosis. N Engl J Med 2018; 379:11-21. https://doi.org/10.1056/NEJMoa1716153.
[52] Han Y H, Kim H J, Na H, Nam M W, Kim J Y, Kim J S, et al. RORα Induces KLF4-Mediated M2 Polarization in the Liver Macrophages that Protect against Nonalcoholic Steatohepatitis. Cell Rep 2017; 20:124-35. https://doi.org/10.1016/j.celrep.2017.06.017.
[53] Toita R, Kawano T, Murata M, Kang J H. Anti-obesity and anti-inflammatory effects of macrophage-targeted interleukin-10-conjugated liposomes in obese mice. Biomaterials 2016; 110:81-8. https://doi.org/10.1016/j.biomaterials.2016.09.018.
[54] Den Boer M A M, Voshol P J, Schroder-van Der Elst J P, Korsheninnikova E, Ouwens D M, Kuipers F, et al. Endogenous interleukin-10 protects against hepatic steatosis but does not improve insulin sensitivity during high-fat feeding in mice. Endocrinology 2006; 147:4553-8. https://doi.org/10.1210/en.2006-0417.
[55] Molinaro A, Koh A, Wu H, Schoeler M, Faggi I, Carreras A, et al. Hepatic expression of Lipopolysaccharide Binding Protein (Lbp) is induced by the gut microbiota through Myd88 and impairs glucose tolerance in mice independent of obesity. Mol Metab 2020:100997. https://doi.org/10.1016/j.molmet.2020.100997.
[56] Zhu L, Brown W C, Cai Q, Krust A, Chambon P, McGuinness O P, et al. Estrogen treatment after ovariectomy protects against fatty liver and may improve pathway-selective insulin resistance. Diabetes 2013; 62:424-34. https://doi.org/10.2337/db11-1718.

### Materials and Methods for Examples 9-13

### Lipid Formulated-siRNA LBP Biosynthesis

In vitro experiments for siRNA LBP selection. Silencer Select Pre-designed siRNA assays si69107, si69108 and si69109 targeting mouse LBP were purchased from Ambion, Life Technologies. siRNA sequences were chemically modified introducing 2′O-methyl RNA bases and UNA monomers to generate UNA oligomers (Integrated DNA Technologies, USA). Chemically modified (CM) siRNA sequences are designated UNA-siRNA throughout the disclosure. As an example, chemically modified si69108 is designated UNA-si69108. Hepa1.6 cells were seeded on 96 well plates the day before transfection at a density of 10⁴ cells per well. Following the vendor recommended protocol, Lipofectamine siRNAmax was used to transfect siRNAs at different concentrations. After 48 hours, cells were washed on PBS and EZCt Cells2Ct Direct Lysis Buffer (Lifeome, USA) was added following the manufacturer's suggested protocol.

Preparation of Lipid Nanoparticle (LNP)-UNA oligomer. Lipid nanoparticles (LNPs) encapsulating LBP UNA oligomers were prepared as described previously (Arcturus Therapeutics, San Diego, CA; 14). Briefly, UNA oligomer was dissolved in 2 mM citrate buffer, pH 3.5. Lipids at the desired molar ratio were dissolved in ethanol. The molar ratio of the constituent lipids was 58% ionizable amino lipid (Arcturus Therapeutics), 7% DSPC (1,2-distearoyl-sn-glycero-3-phosphocholine) (Avanti Polar Lipids, Alabaster, AL, USA), 33.5% cholesterol (Avanti Polar Lipids, Alabaster, AL, USA), and 1.5% DMG-PEG (1,2-Dimyristoyl-sn-glycerol, methoxypolyethylene glycol, PEG chain Molecular weight: 2000) (NOF America Corporation, White Plains, NY). Lipid solution was then combined with UNA oligomer solution using a Nanoassembler™ microfluidic device (Precision NanoSystems Inc., Vancouver, Canada) at a flow rate ratio of 1:3 ethanol:aqueous phases. The mixed material was then diluted with 3× volume of 10 mM Tris buffer, pH 7.4 containing 9% sucrose, reducing the ethanol content to 6.25%. The diluted formulation was then concentrated by tangential flow filtration using hollow fiber membranes (mPES Kros membranes, 100 Kd MWCO, Spectrum Laboratories, Inc., Rancho Dominguez, California), followed by diaflitration against 10 volumes of 10 mM Tris buffer, pH 7.4 containing 9% sucrose. Post diafiltration, formulations were concentrated to the desired siRNA concentration followed by filling into vials and freezing. Formulations were characterized for particle size, siRNA content and encapsulation efficiency. Particle size was determined by dynamic light scattering (ZEN3600, Malvern Instruments). Encapsulation efficiency was calculated by determining unencapsulated siRNA content by measuring the fluorescence upon the addition of RiboGreen (Molecular Probes) to the particles (Fi) and comparing this value to the total RNA content that obtained upon lysis of the particles using 1% Triton X-100 (Ft), where % encapsulation=(Ft−Fi)/Ft×100.

Lentiviral shRNA-Lbp Particle Production

Four different short-hairpin-Lbp (clone set against mouse Lbp, NM_008489.2) primer sequences and random negative control (NC) sequences that did not have targets for any gene were synthesized by Tebu-bio (Tebu-bio, Spain, SL). Lentivirus-targeted Lbp was obtained by cotransfection of shRNA plasmids against Lbp and a combination of packaging and envelope plasmids from Addgene (pCMV-VSV-G and pCMV-dR8.2 dvpr) into HEK293T using LipoD293 transfection reagent following manufacturers' instructions. Lentiviral particles obtained were used to treat Hepa1-6 cell line. Lentiviral effectiveness was confirmed in Hepa1-6 cells.

### Mouse Experiments

### Effect of LNP-siRNA LBP.

Experiment 1. Eight week-old male and female C57/BL6J mice (N=48) were housed during 25 weeks under standard conditions of light (12-hour light/12-hour dark cycle) and temperature (22±1° C.) under the following experimental conditions: i) Non-treated control diet (CD)-fed mice (TD.120455, 3.3 Kcal/g, ENVIGO) for 25 weeks; ii) Non-treated high-fat and high-sucrose diet (HFHS)-fed mice for 25 weeks; iii) Weekly LNP-UNA-si69108 (3 mg/kg) treated HFHS-fed mice for 25 weeks; iv) Weekly LNP-UNA-si69108 (3 mg/kg) treated HFHS-fed mice only in the last 8 weeks. Serum and plasma were collected at 4 and 8 weeks. Body weight and food consumption was reported weekly. Body composition analysis (fat and lean mass) was performed by EchoMRI™ at weeks 4, 14 and 22. At week 25, after overnight fasting, mice were sacrificed by suffocation under sedation. Then, blood serum and plasma, and inguinal (i) and perigonadal (pg) white adipose tissue were collected, immediately frozen in liquid-nitrogen, and stored at −80° C. until processing for RNA or protein analysis.

Experiment 2. Eight week-old female C57/BL6J mice (N=15) were housed during 12 weeks under standard conditions of light (12-hour light/12-hour dark cycle) and temperature (22±1° C.) in the following experimental conditions: i) Non-treated high-fat diet (HFD 60%)-fed mice (TD06414, 5.1 Kcal/g, ENVIGO) during 12 weeks; ii) Weekly LNP-UNA-si69108 (3 mg/kg) treated HFD-fed mice during 12 weeks. Body weight and food consumptions were weekly reported. At week 12, after overnight fasting, mice were sacrificed by suffocation under sedation. Then, liver, iWAT and pgWAT were collected, immediately frozen in liquid-nitrogen, and stored at −80° C. until processing for RNA analysis.

Effects of short-term specific perigonadal WAT and inguinal WAT Lbp gene knockdown using lentiviral shRNA-Lbp particles.

Eight-week-old male and female C57BL/6J mice (N=20) were fed with HFHS with water ad libitum for 4 weeks. Then, lentiviral injection into iWAT and pgWAT was performed at week 12. To inject lentiviral particles into both iWAT and pgWAT, mice were anesthetized by isoflurane before dissection of the skin and body wall. The lentiviral preparation (1×10⁷-8 plaque-forming units in a volume of 100 μl) was injected into the right and left pgWAT and iWAT depot, and was administered first in 6 injections of 10 μl for each pgWAT, and then 4 injections of 8 μl for each iWAT. Each mouse was injected with ˜185 μl of lentiviral preparation. Mice were randomly allocated to the treatment groups (shC group versus shLbp group, n=10 mice/group) and fed with HFHS. At week 17, mice were sacrificed by CO₂ inhalation, and pgWAT and iWAT were rapidly dissected out, frozen in liquid nitrogen, and stored at −80° C. until RNA extraction.

In all mouse experiments, the research was conducted in accordance with the European Guidelines for the Care and Use of Laboratory Animals (directive 2010/63/EU) and was approved by the Ethical Committee for Animal Experimentation of Barcelona Science Park (PCB).

### Human Study

From January 2016 to October 2017, a cross-sectional case-control study was undertaken in the Endocrinology Department of Josep Trueta University Hospital. 63 age- and sex-matched nonobese (BMI 18.5-<30 kg/m²) and 81 obese (BMI≥30 kg/m²) consecutive participants were included, with an age range of 27.2-66.6 years. Exclusion criteria were: previous type 2 diabetes mellitus, chronic inflammatory systemic diseases, acute or chronic infections in the previous month; use of antibiotic, antifungal, antiviral or treatment with proton-pump inhibitors; severe disorders of eating behavior or major psychiatric incidents; neurological diseases, history of trauma or injured brain, language disorders; and excessive alcohol intake (≥40 g OH/day in women or 80 g OH/day in men). Body fat composition was estimated using Bio-electrical impedance analysis (BC-418, Tanita Corporation of America, Illinois, USA). All subjects gave written informed consent, validated and approved by the ethical committee of the Hospital of Girona “Dr Josep Trueta”, after the purpose of the study was explained to them.

### Gene Expression

RNA purification (isolation) was performed using the RNeasy Lipid Tissue Mini Kit (QIAgen, Izasa SA, Barcelona, Spain) and the integrity was checked by the Agilent Bioanalyzer (Agilent Technologies, Palo Alto, CA). Gene expression was assessed by real-time PCR using a LightCycler® 480 Real-Time PCR System (Roche Diagnostics SL, Barcelona, Spain), using TaqMan® technology suitable for relative gene expression quantification. The commercially available and pre-validated TaqMan® primer/probe sets used were as follows: Endogenous control 18S, and target gene mouse lipopolysaccharide binding protein (Lbp, Mm00493139_m1); fatty acid synthase (Fasn, Mm00662319_m1); stearoyl-Coenzyme A desaturase 1 (Scd1, Mm00772290_m1); perilipin 1 (Plin1, Mm00558672_m1); Leptin (Lep, Mm00434759_m1); peroxisome proliferator activated receptor gamma (Ppary, Mm00440940_m1); adiponectin (Adipoq, Mm00456425_m1); fatty acid binding protein 4, adipocyte, (Fabp4, Mm00445880_m1); solute carrier family 2 (facilitated glucose transporter), member 4 (Slc2a4 or Glut4, Mm01245502_m1); lipase, hormone sensitive (Lipe, Mm00495359_m1); monoglyceride lipase (Mgll, Mm00449274_m1); acyl-CoA synthetase long-chain family member 1 (Acs1, Mm00484217_m1); uncoupling protein 1 (mitochondrial, proton carrier) (Ucp1, Mm01244861_m1); neuregulin 4 (Nrg4, Mm00446254_m1); adrenergic receptor, beta 3 (Adrb3, Mm02601819_g1); interleukin 6, (Il6, Mm00446190_m1); tumor necrosis factor (Tnf, Mm00443258_m1); chemokine (C-C motif) ligand 2 (Ccl2, Mm00441242_m1); CD14 antigen (Cd14, Mm01158466_g1); integrin alpha X (Itgax, Mm00498701_m1).

### Western Blot Analysis

Dissected perigonadal WAT was homogenized using an IKA T25 digital ULTRA-TURRAX homogenizer in lysis buffer (50 mmols/l Tris-HCl pH 7.4, 150 mmols/l NaCl, 1.5 mmols/l MgCl2, 1 mmols/l EDTA, 1 mmols/l EGTA, 40 mmols/l β-glycerophosphate, 2 mmols/l Na3VO4, 1 mmols/l PMSF, 1 mmols/l DTT) containing complete protease inhibitor cocktail (Roche Applied Science). Lysates were centrifuged at 1,500×g for 5 minutes at 4° C. to remove intact cells, and protein content was quantified using the Bradford method. For Western blotting, proteins (40 μg) were resolved by SDS-PAGE and transferred to a PVDF membrane (Immobilon; Millipore). Membranes were exposed overnight at 4° C. to primary antibodies anti-UCP1 at 1/1000 dilution (ab10983, Abcam Plc, Cambridge, UK) and anti-β-actin at 1/5000 (A5441, Sigma-Aldrich) both diluted in 1×PBS containing 0.1% Tween-20, following the recommendations of the manufacturer. After secondary antibody incubation (Anti mouse/Rabbit HRP), signals were detected using enhanced chemiluminescence HRP substrate (Millipore) and analyzed with a Luminescent Image Analyzer LAS-3000 (Fujifilm Life Science, Tokyo, Japan).

### Serum/Plasma Measurements

Mouse experiments. Plasma LBP (HK205-02, LBP mouse ELISA kit, Hycult Biotech Inc., PA, USA), leptin (90030) and adiponectin (80569, Crystal Chem, Zaandam, Netherlands).

Human study. Plasma LBP was measured as previously described (6). Serum glucose levels were measured in duplicate by the glucose oxidase method with a Beckman Glucose Analyzer 2 (Beckman Instruments, Brea, CA). The coefficient of variation (CV) was 1.9%. Total serum cholesterol was measured through the reaction of cholesterol esterase/oxidase/peroxidase, using a BM/Hitachi 747. HDL cholesterol was quantified after precipitation with polyethylene glycol at room temperature. Total serum triglycerides were measured through the reaction of glycerol-phosphate-oxidase and peroxidase by routine laboratory tests on a Hitachi 917 instrument (Roche, Mannheim, Germany).

### Statistical Analysis

Statistical analyses were performed using the SPSS 12.0 software. In mouse experiments, all results are expressed as means±SEM. In mouse experiments, differences were tested for statistical significance using Student's unpaired and paired t-tests, and non-parametric tests (Mann-Whitney U test). In human studies, unless otherwise stated, descriptive results of continuous variables are expressed as mean and SD for Gaussian variables or median and interquartile range for non-Gaussian variables. The relation between variables was analyzed by simple correlation (using Spearman's and Pearson's tests). One-factor ANOVA and Student's unpaired t-tests were used to compare clinical variables and plasma LBP concentration based on obesity. Levels of statistical significance were set at p<0.05.

### Example 9

This example describes the effect of plasma LBP depletion on food intake and adiposity.

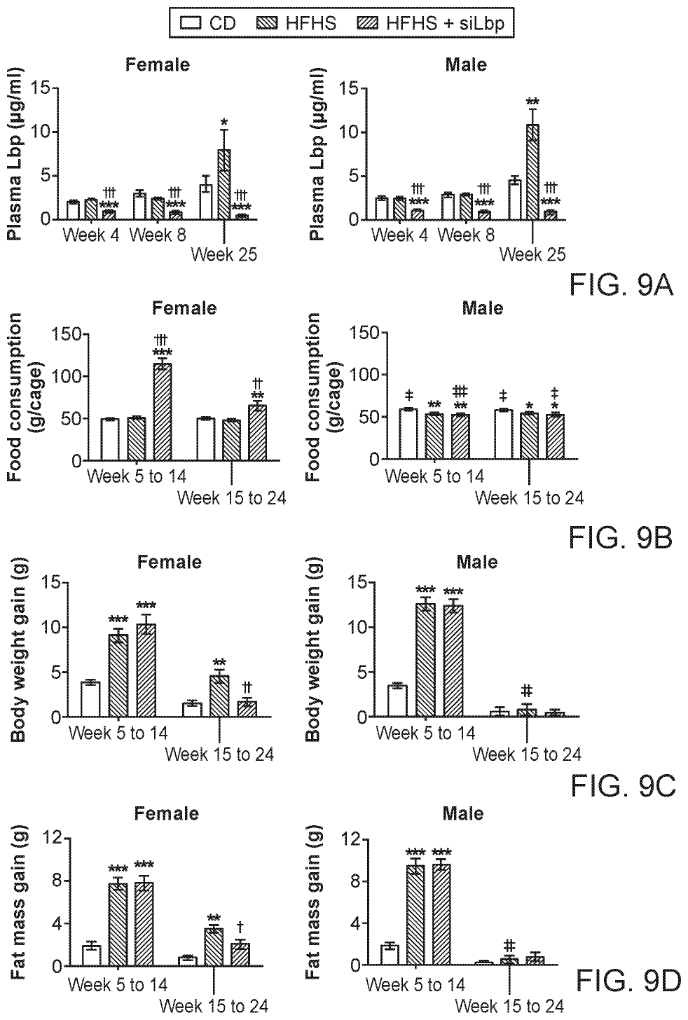

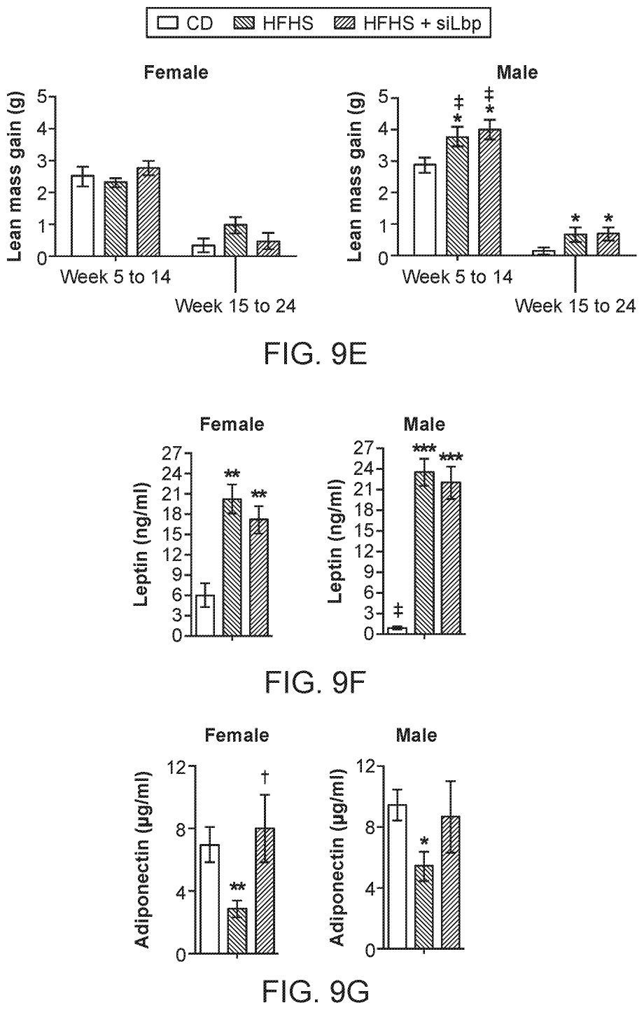

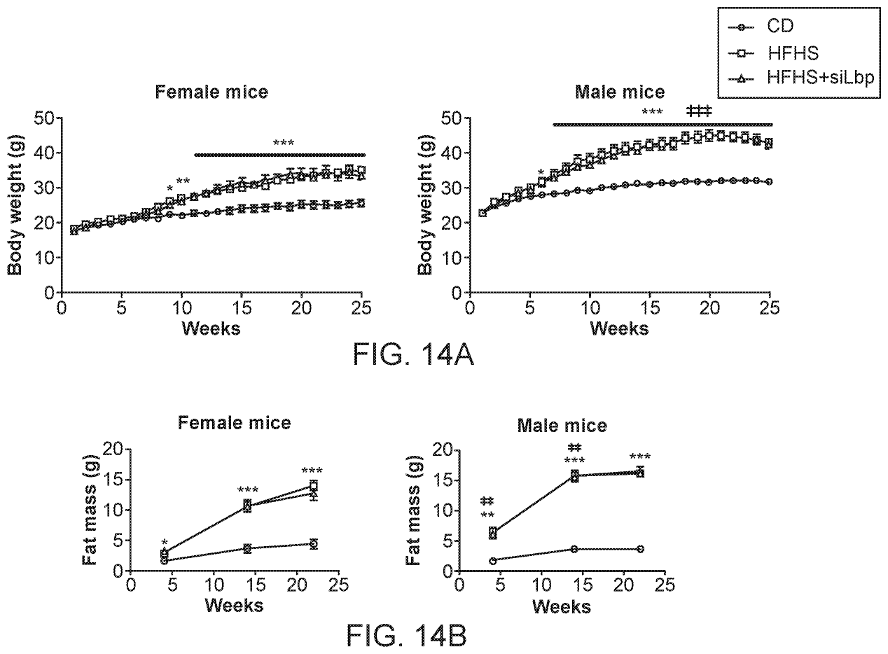

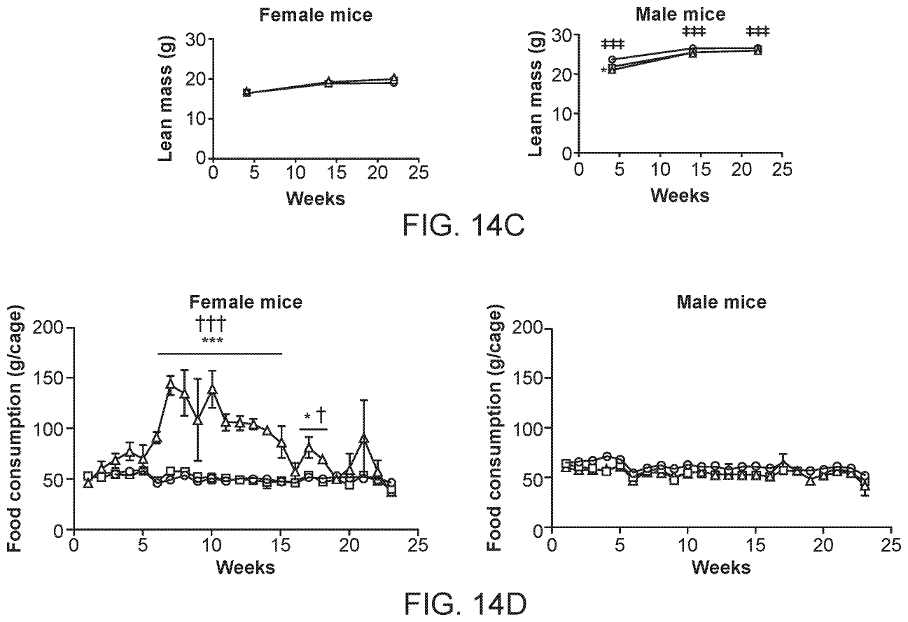

As described above, specific liver LBP gene knockdown (KD) using an LNP-siRNA delivery system resulted in a significant depletion in plasma LBP concentration. Using the same LNP-siRNA delivery system, here, the long-term effect of plasma LBP depletion in mice fed with high fat and high sucrose diet (HFHS) for 6 months was evaluated. LNP-UNA-si69108 injections resulted in a significantly decreased plasma LBP levels (FIG. 9A). Compared to mice fed CD, the weekly record showed a significant increase of body weight and fat mass in both male and female mice given a HFHS diet independent of treatment (FIG. 14A-B). No significant differences in body weight, fat and lean mass (FIG. 14A-C) were found between mice with (HFHS+L) or without (HFHS) LNP-UNA-si69108 treatment.

Differences in food consumption based on gender were detected during the weekly evaluations (FIG. 14D). Food consumption decreased in males but not in females on HFHS. However, when treated with LNP-UNA-si69108, food consumption increased in females while remaining decreased in males (FIG. 9B, FIG. 14D). Interestingly, the maximum increase in food consumption in females in the HFHS+L group was found in the period from week 5 to week 15 (2.3 fold-increase, p<0.0001) without significant effects on weight and fat mass gain (FIG. 9C-E). Although still significant, the increase of food consumption was attenuated during the period from weeks 15 to 25, while weight gain and fat mass gain were significantly reduced (FIG. 9C-E).

In contrast to females, in males, food consumption, body weight gain and fat and lean mass gain on a HFHS diet were not affected by LNP-UNA-si69108 (FIG. 9B-E) treatment. In summary, these results show that reduction of plasma LBP levels by LNP-UNA-si69108 increased food consumption and reduced weight gain and fat mass gain in female, but not in male, HFHS-fed mice.

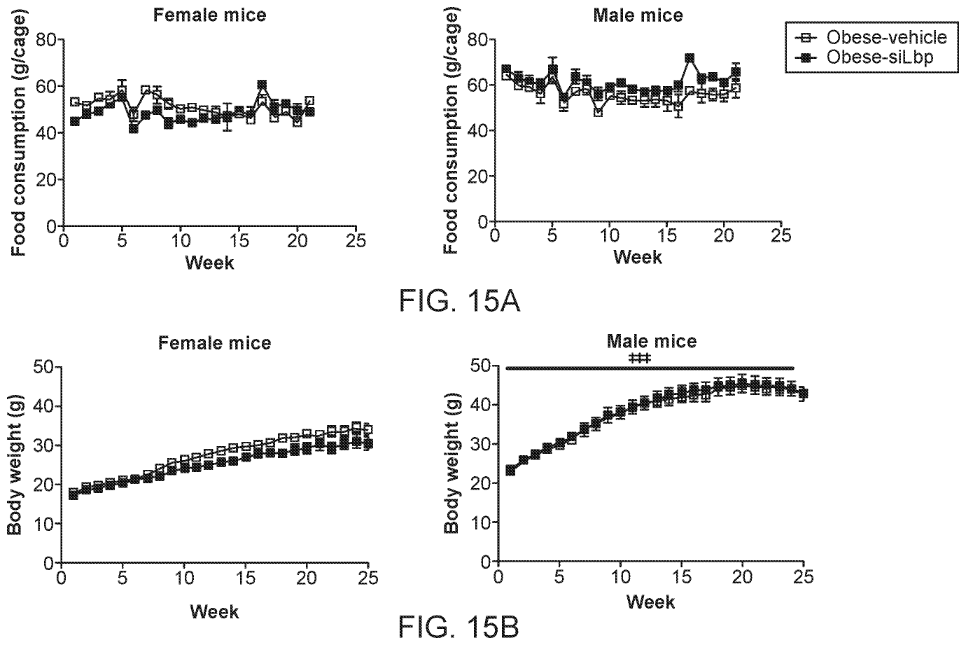

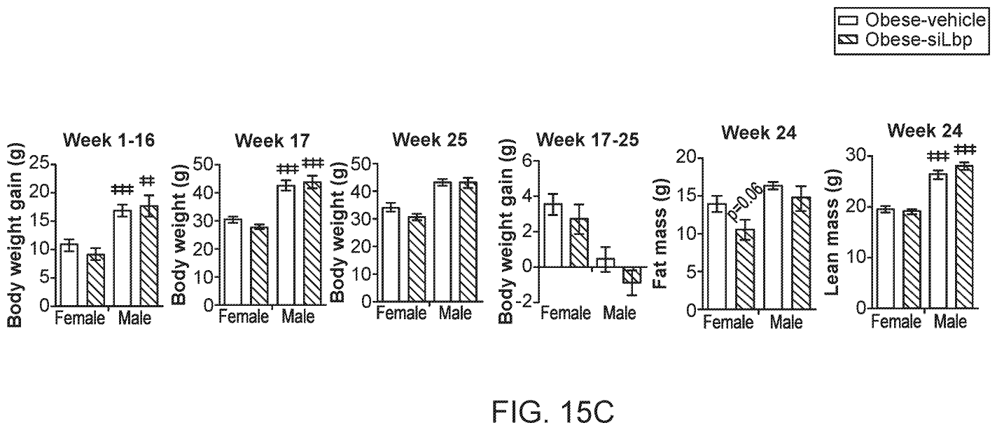

The impact of plasma LBP depletion on food consumption and adiposity in HFHS-induced obese mice treated with LNP-UNA-si69108 for 8 weeks was also examined. In male obese mice, this intervention resulted in increased food consumption without significant effects on body weight, fat and lean mass (FIG. 15A-C). In female obese mice, 8 week-LNP-UNA-si69108 administration did not have any apparent effects on food consumption, body weight and lean mass, but tended to reduce fat mass (p=0.08) (FIG. 15A-C). At week 24, body weight and lean but not fat mass were significantly increased in male as compared to female mice (FIG. 15C).

These results show that plasma LBP depletion impacted food intake and adiposity in female mice.

### Example 10

This example describes the effect of plasma LBP depletion on circulating adiponectin levels.

Together with fat mass gain, leptin is an important circulating fatness marker (15). Leptin increased progressively in the HFHS group without significant differences seen in the HFHS+L group (FIG. 9G). Similar to body weight and fat mass, leptin levels were significantly increased in male as compared to female mice at week 4 and 8 (FIG. 9F). In contrast, circulating adiponectin, which is negatively associated with obesity (15), was significantly decreased in the HFHS group at week 25 (FIG. 9G). Interestingly, in both female and male mice, LNP-UNA-si69108 treatment restored adiponectin levels under obesogenic conditions, with plasma adiponectin levels in the HFHS+L group similar to levels seen in the CD group at week 25 (FIG. 9G).

These results show that plasma LBP depletion improved circulating adiponectin levels.

### Example 11

This example describes the effect of plasma LBP depletion on adipogenesis and thermogenesis.

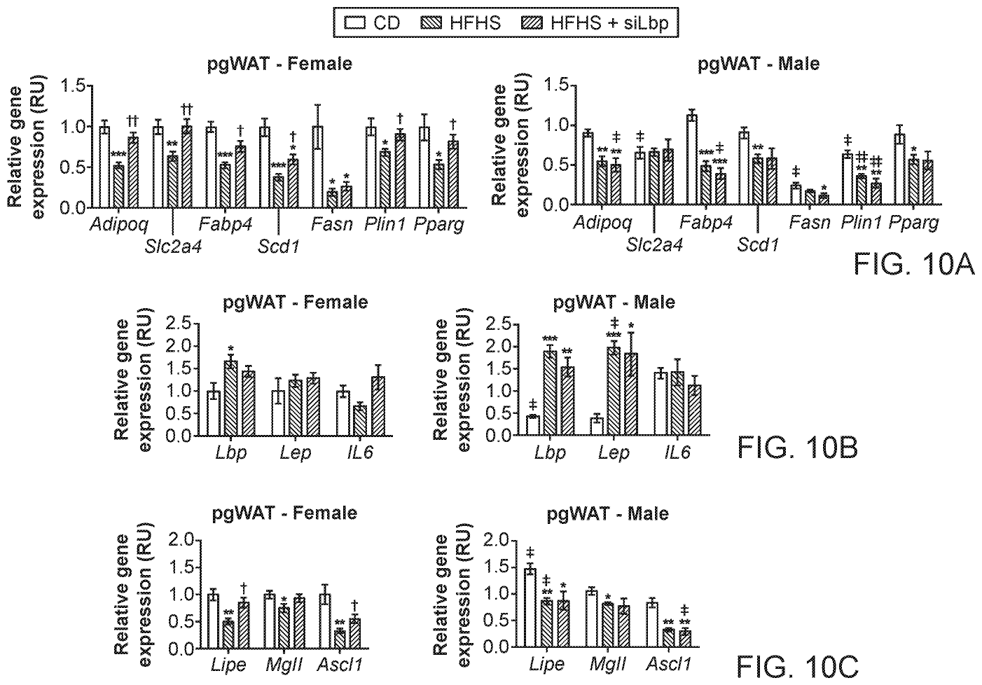

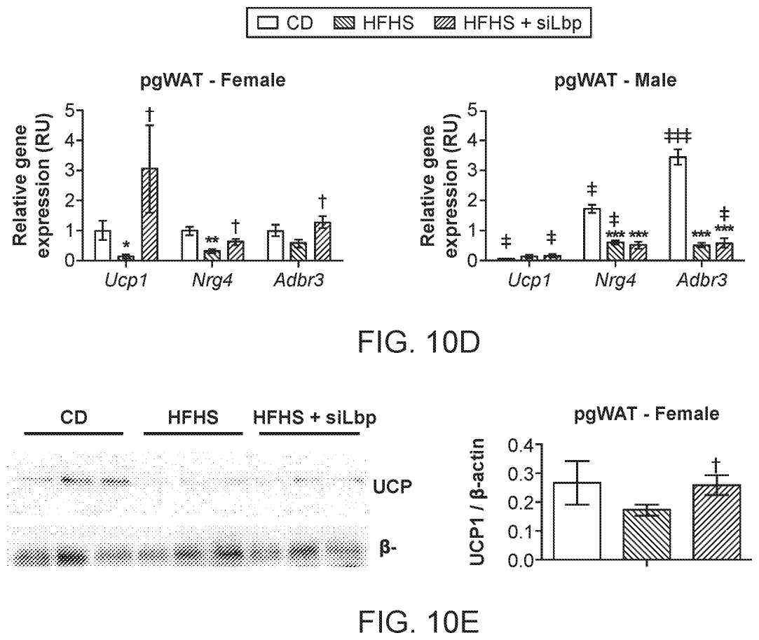

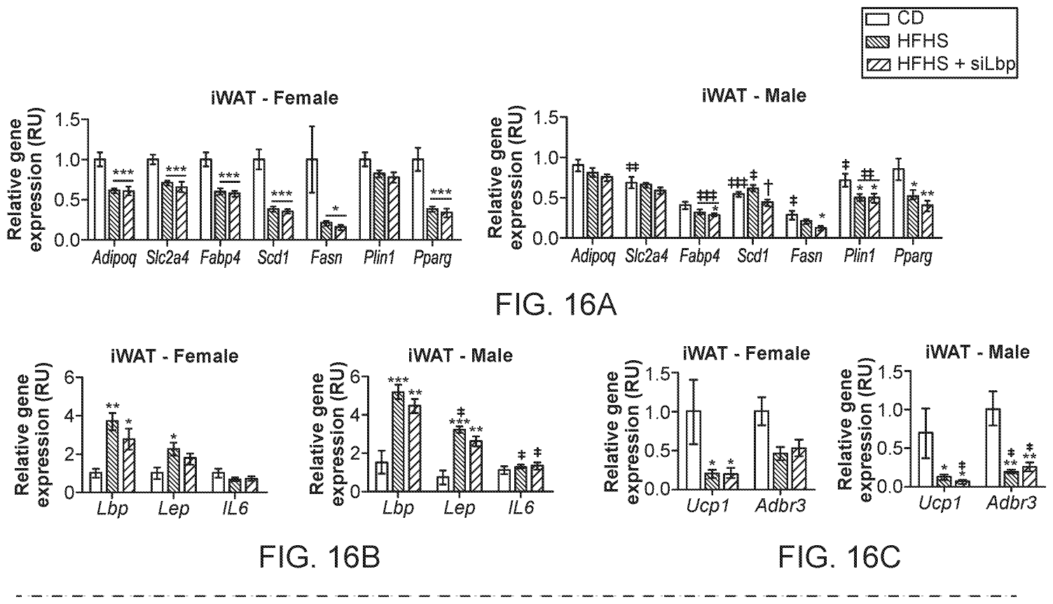

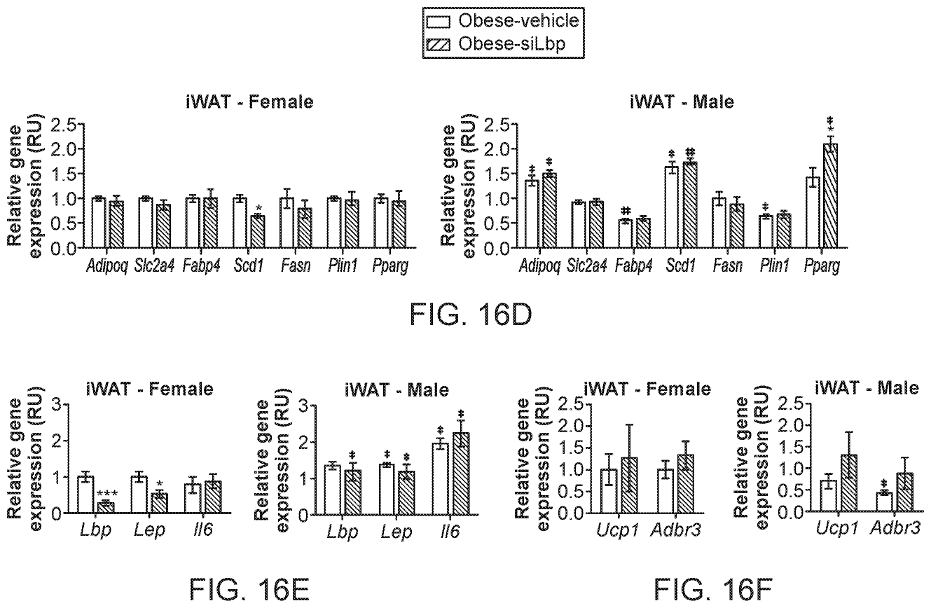

Signs of improved adipose tissue function were observed in female mice fed a standard diet along with increased lipid storage capacity-related gene expression (including Slc2a4, Fabp4, Scd1, Fasn and Plin1 in inguinal (i) WAT and Slc2a4, Fasn and Plin1 in perigonadal (pg) WAT) (FIG. 10A and FIG. 16 A, D). These observations were in concordance with the healthy metabolic phenotype found in females compared to males fed a standard diet (FIG. 10 and FIG. 16). However, signs of adipose tissue dysfunction were observed in females fed with HFHS, resulting in similar adipogenic gene expression as compared to male mice in both iWAT and pgWAT (FIG. 10 and FIG. 16). In addition, confirming the negative effects of HFHS on WAT physiology, increased Lbp and Lep mRNA levels (FIG. 10 B) and decreased browning-related genes (Ucp1 and Adbr3) were observed (FIG. 10 D).

In male and female iWAT and male pgWAT, 6 months of LNP-UNA-si69108 administration did not have any apparent effect on adipose tissue gene expression (FIG. 10A-E and FIG. 16A-C). By contrast in female pgWAT, treatment with LNP-siRNA resulted in significantly increased adipogenic (Adipoq, Slc2a4, Fabp4, Scd1, Plin1, Pparg), lipolitic (Lipe, Mgll, Acsl1), and browning (Ucp1, Nrg4, Adbr3) gene expression and UCP1 protein levels as compared to untreated mice fed a control diet (FIG. 10A-E).

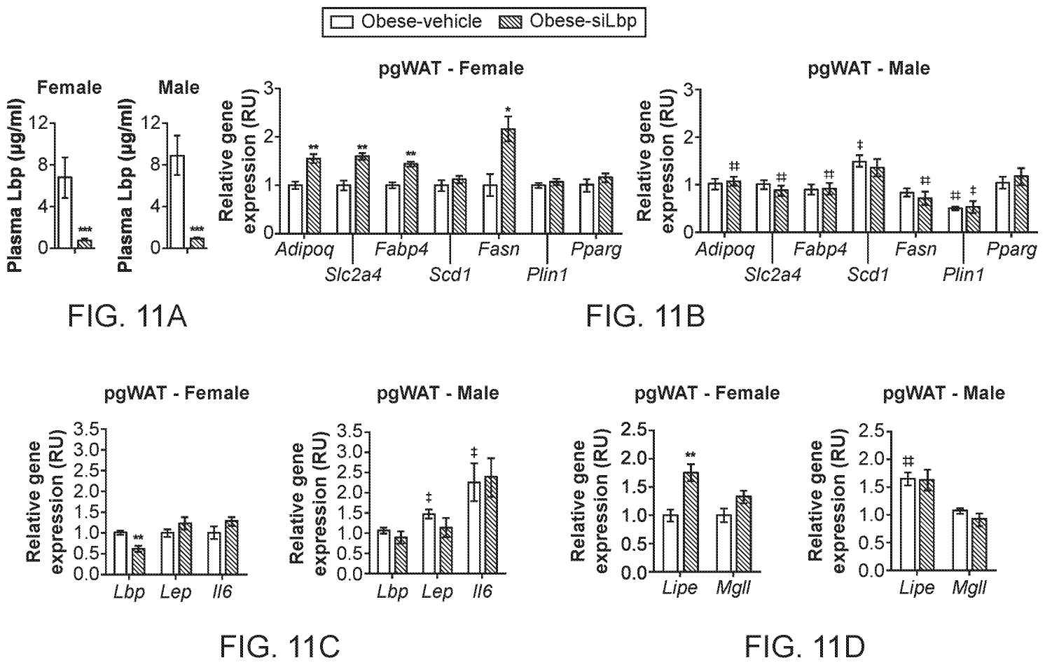

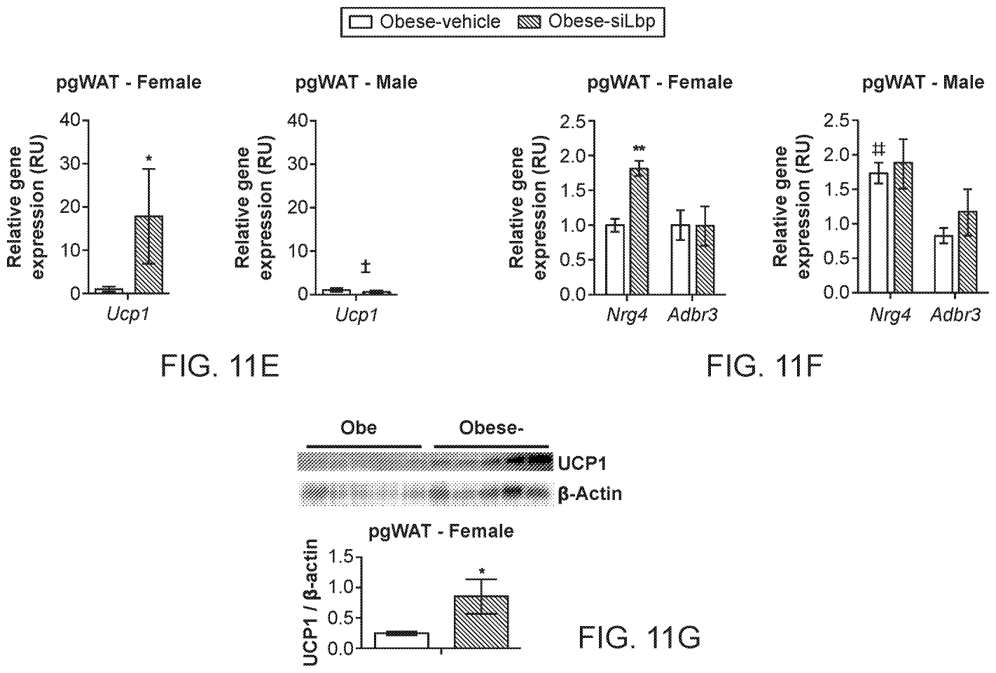

In 8 week-LNP-UNA-si69108 treated obese mice, similar results were seen, with Adipoq, Slc2a4, Fabp4, Fasn, Lipe, Mgll, Ucp1 and Nrg4 gene expression and UCP1 protein levels significantly increased only in female pgWAT (FIG. 11A-G and FIG. 16D-F). Together, these data showed that LNP-UNA-si69108 treatment can partially restore to normal levels a HFHS-induced gene expression profile in visceral (perigonadal) adipose tissue in female mice.

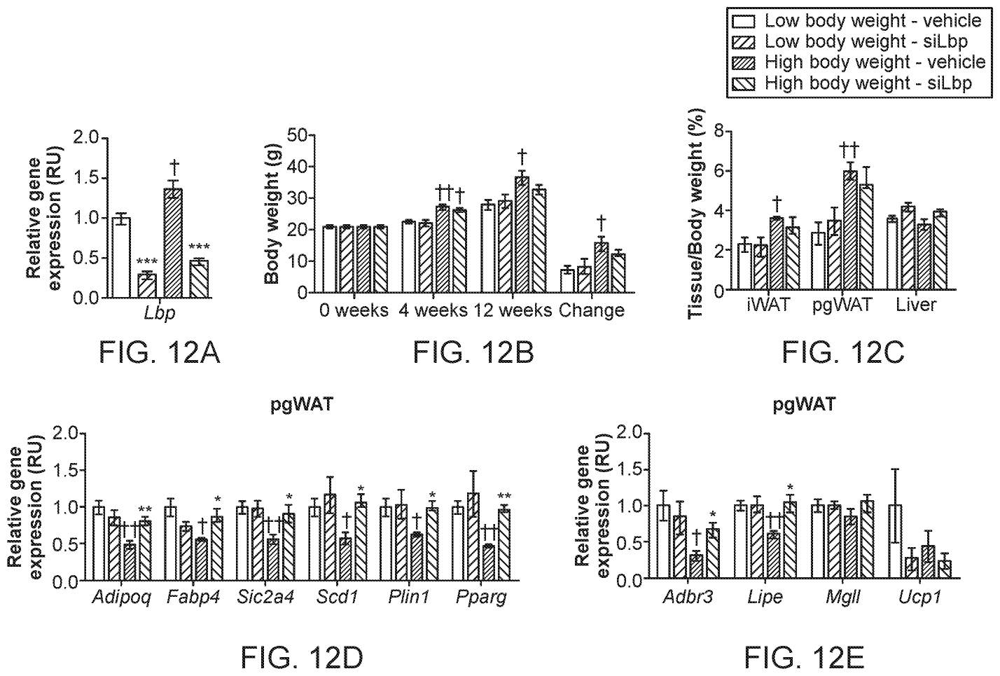

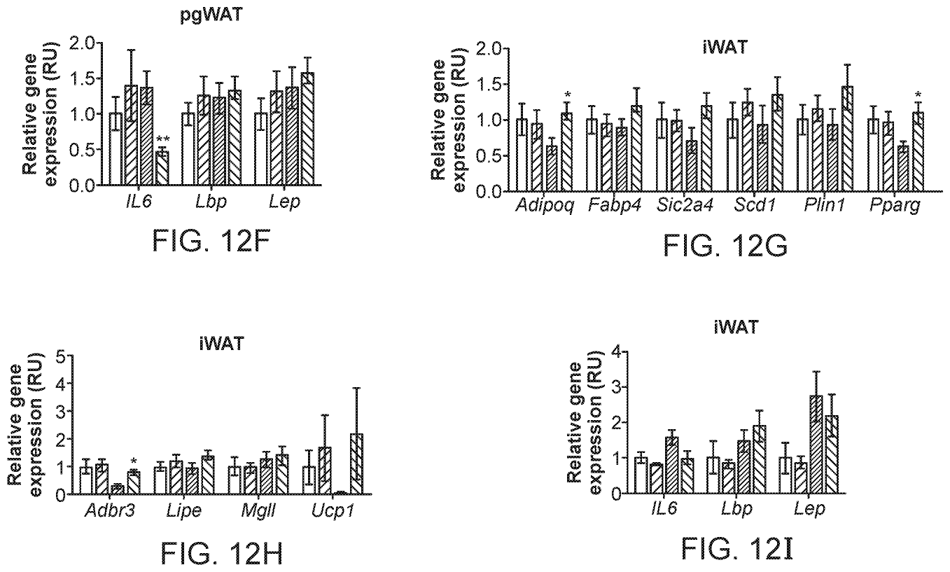

In a further experiment, the short-term effect of plasma LBP depletion in female mice fed a high fat diet (HFD, 60%) for 3 months was also evaluated. Of interest, in mice with high body weight at week 4, LNP-UNA-si69108 administration attenuated weight gain and fat accretion in parallel with improved expression of Adbr3 and adipogenic-related genes, including Adipoq, Fabp4, Scd1, Plin1, Pparg and Lipe in pgWAT and Adipoq and Pparg in iWAT (FIG. 12A-I). Again, strong effects of LNP-UNA-si69108 treatment were observed in pgWAT (FIG. 12D-F).

These results show that plasma LBP depletion improved adipogenesis and thermogenesis in visceral fat depots in females.

### Example 12

This example describes the effect of short-term specific iWAT and pgWAT Lbp gene knockdown on the expression of fat accretion-related genes.

Specific WAT Lbp gene knockdown was performed by injecting lentiviral particles delivering shRNA scramble and shRNA against Lbp mRNA directly into iWAT and pgWAT using a surgical method of lentiviral particle administration as detailed above (Materials and Methods for Examples 9-13).

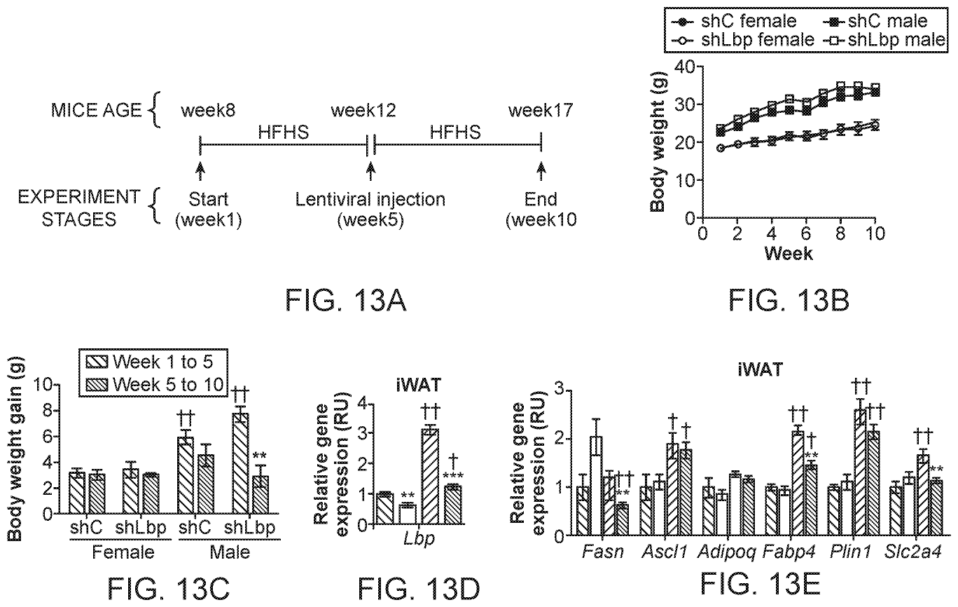

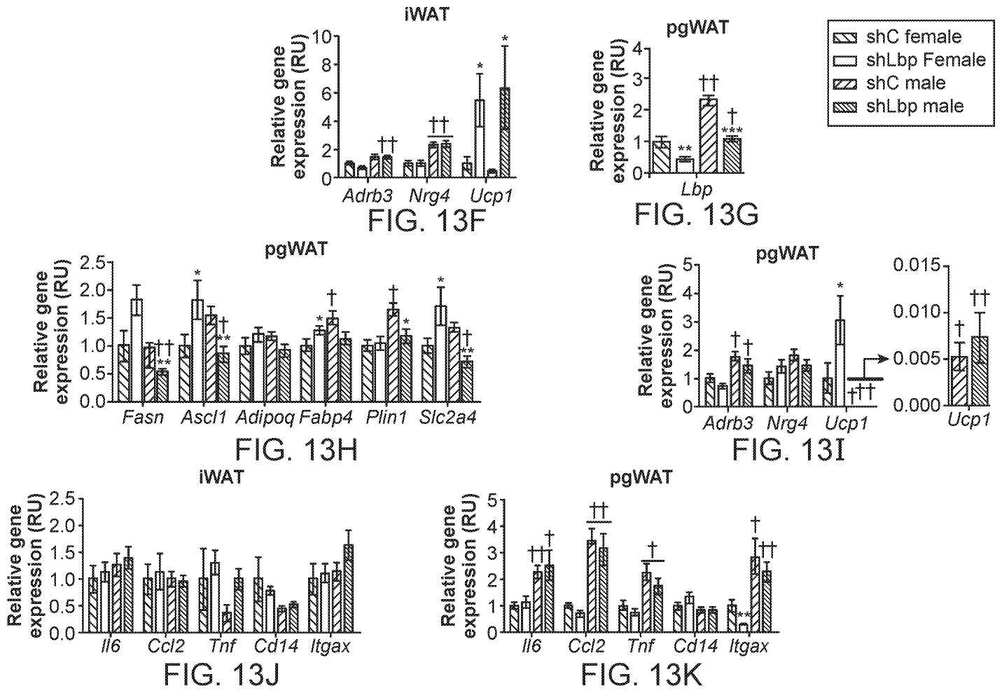

In a pilot study, an effective gene knockdown was observed for 5 weeks following a single surgical administration, but weeks later the effect was lost. Thus, in the present study, a single surgical administration of lentiviral particles was used to evaluate the short-term effects of WAT Lbp gene knockdown in adipose tissue physiology in 12 week-aged mice fed with HFHS (FIG. 13A). Sexual dimorphism was observed in this experiment. Increased body weight gain was observed in male as compared to female mice (FIG. 13B-C), indicating increased fat accretion in males during this period. Similar to body weight and fat accretion-related gene expression (Fabp4, Plin1, Slc2a4), Lbp gene expression was increased in male as compared to female mice in both iWAT and pgWAT (FIG. 13D-I). Specific iWAT Lbp gene KD (65% in males and 37% in females, p<0.0001, FIG. 13D) and pgWAT Lbp gene KD (70% in males and 55% in females, p<0.0001, FIG. 13G) resulted in a significant decrease in body weight gain in male, but not in female mice (FIG. 13C). Specific iWAT Lbp gene KD also resulted in decreased fat accretion-related gene expression (including Fasn, Fabp4, Plin1, Slc2a4) in male mice, whereas no significant changes in expression of these genes were observed in female mice (FIG. 13E-F). Notably, iWAT Lbp gene KD increased Ucp1 mRNA levels in this fat depot in both male and female mice (FIG. 13F). Similar to iWAT, specific pgWAT Lbp gene KD resulted in decreased Fasn, Fabp4, Plin1, Slc2a4 gene expression in male mice (FIG. 13H) and increased Ucp1 mRNA levels only in female mice (FIG. 13I). Interestingly, Ucp1 mRNA levels were increased 500-fold in female as compared to male pgWAT (p<0.0001, FIG. 13I), showing sexual dimporphism in pgWAT Ucp1 gene expression in young mice, which may explain the resistance to fat accretion and body weight gain during the early months of life in female mice (FIG. 13C). Lentiviral particle-mediated shRNA delivery targeting Lbp did not have any apparent effects on adipose tissue inflammatory markers (except for a significant reduction in pgWAT Itgax mRNA in females, FIG. 13J-K).

These results show that short-term specific iWAT and pgWAT Lbp gene knockdown attenuated expression of fat accretion-related genes and promoted UCP-1 activity.

### Example 13

This example describes the correlation of LBP concentration with percent fat mass and circulating leptin.

To investigate the importance of LBP in fat accretion in humans, plasma LBP concentration was correlated with adiposity measures, including BMI, percent fat and lean mass, total body fat, waist circumference and circulating leptin in non-obese and obese participants (Table 3). Interestingly, plasma LBP levels were positively correlated with total body fat and leptin in non-obese and morbidly obese participants (Table 3). Importantly, plasma LBP levels were positively correlated with BMI, percent fat mass and waist circumference, but negatively with percent lean mass, in morbidly obese participants.

TABLE 3
Anthropometric and clinical parameters according to obesity, and bivariate correlations between
plasma LBP and anthropometric and clinical parameters in non-obese and obese participants.

| | | | | Non-obese | | Obese | |
|---|---|---|---|---|---|---|---|
| | Non-obese | Obese | P | r | p | r | P |
| N (men/women) | 63 (20/43) | 81 (24/57) | | | | | |
| Age (years) | 49.7 ± 10.6 | 45.8 ± 10.5 | 0.03 | 0.12 | 0.3 | −0.12 | 0.3 |
| BMI (kg/m2) | 24.9 ± 2.6 | 43.6 ± 6.8 | <0.0001 | −0.10 | 0.4 | 0.35 | 0.002 |
| Percent fat mass (%) | 25.4 ± 6.3 | 43.2 ± 5.3 | <0.0001 | 0.17 | 0.1 | 0.45 | <0.0001 |
| Percent lean mass (%) | 74.2 ± 6.9 | 56.8 ± 5.3 | <0.0001 | −0.19 | 0.1 | −0.44 | <0.0001 |
| Percent total fat (%) | 32.9 ± 7.4 | 50.1 ± 5.2 | <0.0001 | 0.27 | 0.03 | 0.39 | 0.001 |
| Waist circumference (cm) | 89.3 ± 9.9 | 126.3 ± 14.4 | <0.0001 | −0.04 | 0.7 | 0.23 | 0.04 |
| Total cholesterol (mg/dl) | 201.7 ± 40.7 | 194.1 ± 41.2 | 0.2 | 0.08 | 0.6 | −0.12 | 0.3 |
| HDL cholesterol (mg/dl) | 65.9 ± 19.4 | 51.1 ± 12.6 | <0.0001 | 0.14 | 0.3 | −0.10 | 0.3 |
| LDL cholesterol (mg/dl) | 120.3 ± 34.1 | 119.7 ± 34.3 | 0.9 | 0.09 | 0.5 | −0.06 | 0.6 |
| Fasting triglycerides (mg/dl) | 79 | 113 | 0.007 | 0.07 | 0.6 | −0.08 | 0.4 |
| | (58-100) | (70-149) | | | | | |
| Fasting glucose (mg/dl) | 94.9 ± 13.1 | 96.4 ± 11.1 | 0.5 | 0.03 | 0.8 | −0.12 | 0.3 |
| Leptin (ng/ml) | 2.35 | 23.8 | <0.0001 | 0.34 | 0.009 | 0.41 | <0.0001 |
| | (0.46-6.48) | (15.7-32.1) | | | | | |
| LBP (μg/ml) | 12.1 ± 3.5 | 17.9 ± 4.8 | <0.0001 | — | — | — | — |

These results show that plasma LBP concentration was associated with percent fat mass and circulating leptin in non-obese and morbidly obese subjects.

### Discussion of Examples 9-13

Obesity, a worldwide epidemic caused by disturbed energy balance (increased food energy intake and/or decreased energy expenditure) and characterized by adipose tissue enlargement and increased body fat accretion, is an important factor in the progression of metabolic diseases, including type 2 diabetes, dyslipidemia, arterial hypertension, ischemic heart disease, non-alcoholic fatty liver disease and some types of cancer, and contributing to the overall burden of disease worldwide (1,2). LPS-binding protein (LBP) has been identified as a component of innate immunity response associated with obesity and insulin resistance (3-9). Observations in humans (10) and experiments in human and 3T3-L1 adipocytes (11,12) and in Lbp knockout (KO) mice (13) pointed to a possible role of Lbp in fat accretion.

To the best of available knowledge, this is the first study that investigated the specific impact of plasma and WAT LBP on obesity-associated adipose tissue dysfunction. First, this study demonstrated that LNP-UNA-si69108 was an accurate and effective way to deplete plasma LBP concentration. Whereas no significant effects of long-term (6 months) LNP-UNA-si69108 administration was found on food consumption and weight and fat mass gain in male mice, in females this intervention resulted in increased food consumption, but decreased fat mass gain. In addition, in obese female mice, short-term (8 weeks) LNP-UNA-si69108 administration tended to decrease fat mass, without significant differences in food consumption. These data indicate that this intervention attenuated fat mass gain in female mice. Without being limited by theory, mechanistically, these beneficial effects may be explained by the reduction of the negative impact of LBP on browning of WAT (13). Studies described herein demonstrated that plasma LBP depletion (by LNP-UNA-si69108 treatment) enhanced the thermogenic program in female perigonadal fat, increasing expression of browning markers (Ucp1), factors that promote browning (Aidbr3, Nrg4) and transporters that facilitate the capture of lipid energy substrates (Lipe and Slc2a4). Consistent with data described herein:
i) Induction of browning (cold or β3-adrenergic agonist CL316243) correlated with the reduction of LBP in both inguinal and visceral (or epididymal, which is less prone to browning) WAT, but not in interscapular BAT (13), indicating that the relationship between Lbp and thermogenesis was specific to WAT.
ii) Lbp repressed browning in a cell-autonomous manner (13).
iii) Lbp KO mice were protected against high fat diet-body weight and fat mass gain that correlated with a marked induction of browning gene markers (Ucp1 and Dio2) and an increased number of multivacuolar adipocytes in inguinal WAT, and explants of inguinal WAT from Lbp-null mice had an increased rate of glucose oxidation (13).
iv) Lbp gene knockdown increased mitochondrial respiration and prevented mitochondrial dysfunction in fully-differentiated 3T3-L1 adipocytes (11).

Taken together, these studies (11,13) and data provided herein show that LBP depletion is a new endogenous way to promote WAT browning.

Data provided herein also suggested that reduced plasma LBP levels might be a browning stimulus that enhances female sex hormone-induced sympathetic signalling sensitivity in female gonadal WAT (16-17), consistent with findings that female sex hormones can specifically enhance the thermogenic response in gonadal, but not inguinal, WAT in response to browning stimuli (16). Experiments in adipocytes demonstrated that estrogen reduced alpha 2 adrenergic receptor expression in parallel with increased beta 3 adrenergic receptor availability, sensitizing adipocytes to sympathetic signalling and promoting browning in these cells (17).

Without being limited by theory, the specific thermogenic effect of LNP-siLBP treatment might explain the high food consumption and weight gain resistance observed in females, but not in males. These antiobesity effects of LNP-siLBP administration might also explain the significant improvement of adipogenic gene expression in females, indicating an important improvement in perigonadal WAT functionality. In line with these findings, plasma LBP depletion prevented the reduction of circulating adiponectin levels observed in mice fed with HFHS. Adiponectin is a specific marker of adipose tissue physiology (15). A negative impact of Lbp on adipocyte differentiation has been previously demonstrated in several in vitro experimental models, including bone marrow mesenchymal stem cells, 3T3-L1 and 3T3-F442A cell lines, and human preadipocytes. However described herein is the first in vivo study demonstrating that plasma Lbp depletion prevented obesity-associated dysfunctional adipogenesis in visceral fat depots.

Without being limited by theory, data provided herein also suggested that anti-obesity effects observed in LBP KO mice (13) might be caused by specific WAT Lbp gene depletion-induced browning. Confirming WAT LBP as a WAT browning repressor, specific iWAT and pgWAT Lbp gene knockdown resulted in increased protein levels of iWAT Ucp1 in male and female mice, and in increased levels of pgWAT Ucp1 only in female mice. In all experiments, including mice aged 8 months (FIG. 10D) and mice aged 4 months (FIG. 13I), increased Ucp1 mRNA levels were found in female mice but not male mice, but this sexual dimorphism was enhanced in young mice (4 months), with Ucp1 mRNA levels increased 500-fold in female mice as compared to levels in male mice (p<0.0001, FIG. 13I). Without being limited by theory, this accentuated pgWAT browing activity in young female mice might explain the enhanced food consumption seen upon 6 month-treatment with LNP-UNA-si69108, mainly in 13-23 week old mice, and the resistance to fat accretion and body weight gain in female mice during the early months of life. In contrast, the short-term effect of plasma LBP depletion in female mice fed with high fat diet (HFD, 60%) for 3 months did not modify pgWAT or iWAT Ucp1, but resulted in increased Adbr3 mRNA levels in both fat depots, suggesting that high β-adrenergic response capacity associated with plasma Lbp depletion in young mice might underlie the enhanced Ucp1 levels observed in 8 month old mice, when plasma Lbp levels were depleted. In agreement with this possibility, age-associated loss of WAT browning was associated with decreased expression of the Adbr3 gene, further supporting reduced local adrenergic tone as a potential mechanism.

Furthermore, increased iWAT and pgWAT Lbp gene expression was observed in male young mice that correlated with body weight and expression of fat accretion-related genes. Importantly, in these mice, specific iWAT and pgWAT Lbp gene KD resulted in decreased fat accretion-related gene expression as well as decreased body weight gain. Without being limited by theory, these data indicated that adipose tissue Lbp might exert a possible role in obesity-associated fat accretion, as suggested in previous observational studies in humans and mice (6,10,13). Findings in humans described herein confirmed the association between plasma LBP concentration and fat accretion-related parameters (BMI, fat mass or circulating leptin) even in morbidly obese participants, strengthening the relationship between adiposity and LBP.

In conclusion, studies described herein provide evidence regarding the relevance of LBP in fat accretion, and suggest plasma LBP depletion in obese mice using an LNP-siRNA delivery system (14) as a novel therapeutic approach for the prevention of obesity-associated fat accretion and improvement of adipose tissue physiology in female mice. Further experiments may further contribute to understanding sex-dependent effects of plasma LBP depletion.

### REFERENCES FOR EXAMPLES 9-13

1—Kusminski C M, Bickel P E, Scherer P E. Targeting adipose tissue in the treatment of obesity-associated diabetes. Nat Rev Drug Discov. 2016 September; 15(9):639-660.
2—Chouchani E T, Kajimura S. Metabolic adaptation and maladaptation in adipose tissue. Nat Metab. 2019 February; 1(2):189-200.
3—Kheirandish-Gozal L, Peris E, Wang Y, Tamae Kakazu M, Khalyfa A, Carreras A, Gozal D. Lipopolysaccharide-binding protein plasma levels in children: effects of obstructive sleep apnea and obesity. J Clin Endocrinol Metab. 2014 February; 99(2):656-63.
4—Sun L, Yu Z, Ye X, Zou S, Li H, Yu D, Wu H, Chen Y, Dore J, Clement K, Hu F B, Lin X. A marker of endotoxemia is associated with obesity and related metabolic disorders in apparently healthy Chinese. Diabetes Care. 2010 September; 33(9):1925-32.
5—Moreno-Navarrete J M, Ortega F, Serino M, Luche E, Waget A, Pardo G, Salvador J, Ricart W, Frühbeck G, Burcelin R, Fernindez-Real J M. Circulating lipopolysaccharide-binding protein (LBP) as a marker of obesity-related insulin resistance. Int J Obes (Lond). 2012 November; 36(11):1442-9.
6—Serrano M, Moreno-Navarrete J M, Puig J, Moreno M, Guerra E, Ortega F, Xifra G, Ricart W, Fernindez-Real J M. Serum lipopolysaccharide-binding protein as a marker of atherosclerosis. Atherosclerosis. 2013 October; 230(2):223-7.
7—Clemente-Postigo M, Roca-Rodriguez Mdel M, Camargo A, Ocana-Wilhelmi L, Cardona F, Tinahones F J. Lipopolysaccharide and lipopolysaccharide-binding protein levels and their relationship to early metabolic improvement after bariatric surgery. Surg Obes Relat Dis. 2015 July-August; 11(4):933-9.
8—Tilves C M, Zmuda J M, Kuipers A L, Nestlerode C S, Evans R W, Bunker C H, Patrick A L, Miljkovic I. Association of Lipopolysaccharide-Binding Protein With Aging-Related Adiposity Change and Prediabetes Among African Ancestry Men. Diabetes Care. 2016 March; 39(3):385-91.
9—Liu X, Lu L, Yao P, Ma Y, Wang F, Jin Q, Ye X, Li H, Hu F B, Sun L, Lin X. Lipopolysaccharide binding protein, obesity status and incidence of metabolic syndrome: a prospective study among middle-aged and older Chinese. Diabetologia. 2014 September; 57(9):1834-41.
10—Moreno-Navarrete J M, Escote X, Ortega F, Serino M, Campbell M, Michalski M C, Laville M, Xifra G, Luche E, Domingo P, Sabater M, Pardo G, Waget A, Salvador J, Giralt M, Rodriguez-Hermosa J I, Camps M, Kolditz C I, Viguerie N, Galitzky J, Decaunes P, Ricart W, Frühbeck G, Villarroya F, Mingrone G, Langin D, Zorzano A, Vidal H, Vendrell J, Burcelin R, Vidal-Puig A, Fernindez-Real J M. A role for adipocyte-derived lipopolysaccharide-binding protein in inflammation- and obesity-associated adipose tissue dysfunction. Diabetologia. 2013 November; 56(11):2524-37.
11—Moreno-Navarrete J M, Jove M, Padró T, Boada J, Ortega F, Ricart W, Pamplona R, Badimón L, Portero-Otin M, Fernindez-Real J M. Adipocyte lipopolysaccharide binding protein (LBP) is linked to a specific lipidomic signature. Obesity (Silver Spring). 2017 February; 25(2):391-400.
12—Moreno-Navarrete J M, Escote X, Ortega F, Camps M, Ricart W, Zorzano A, Vendrell J, Vidal-Puig A, Fernindez-Real J M. Lipopolysaccharide binding protein is an adipokine involved in the resilience of the mouse adipocyte to inflammation. Diabetologia. 2015 October; 58(10):2424-34.
13—Gavaldá-Navarro A, Moreno-Navarrete J M, Quesada-López T, Cairó M, Giralt M, Fernindez-Real J M, Villarroya F. Lipopolysaccharide-binding protein is a negative regulator of adipose tissue browning in mice and humans. Diabetologia. 2016 October; 59(10):2208-18.
14—Ramaswamy S, Tonnu N, Tachikawa K, Limphong P, Vega J B, Karmali P P, Chivukula P, Verma I M. Systemic delivery of factor IX messenger RNA for protein replacement therapy. Proc Natl Acad Sci USA. 2017 Mar. 7; 114(10):E1941-E1950.
15—Frühbeck G, Catalán V, Rodríguez A, Ramírez B, Becerril S, Salvador J, Colina I, Gómez-Ambrosi J. Adiponectin-leptin Ratio is a Functional Biomarker of Adipose Tissue Inflammation. Nutrients. 2019 Feb. 22; 11(2).
16—Kim S N, Jung Y S, Kwon H J, Seong J K, Granneman J G, Lee Y H. Sex differences in sympathetic innervation and browning of white adipose tissue of mice. Biol Sex Differ. 2016 Dec. 9; 7:67.
17—Monjo M, Rodríguez A M, Palou A, Roca P. Direct effects of testosterone, 17 beta-estradiol, and progesterone on adrenergic regulation in cultured brown adipocytes: potential mechanism for gender-dependent thermogenesis. Endocrinology. 2003 November; 144(11):4923-30.

SEQUENCES
SEQ ID NO: 1
UNA-G/*/mUrUmUrAmArGmGrGrUrGmArAmArUmUrUmUrU/*/UNA-U
SEQ ID NO: 2
mArAmArAmArUmUrUmCrAmCmCmCrUmUrAmArAmC/UNA-U
SEQ ID NO: 3
UNA-G/*/mCrCmUrAmCrCmUrGrArGmGrAmCrAmGrUmArA/*/UNA-U
SEQ ID NO: 4
mUrUmArCmUrGmUrCmCrUmCmAmGrGmUrAmGrGmC/UNA-U
SEQ ID NO: 5
UNA-G/*/mArUmGrUmGrCmArCrArUmArUmCrAmGrGmArA/*/UNA-U
SEQ ID NO: 6
mUrUmCrCmUrGmArUmArUmGmUmGrCmArCmArUmC/UNA-U
SEQ ID NO: 7
UNA-G/*/mUrUmUrAmArGmGrGrUrGmArAmArUmUrUmUrU/*/UNA-U/*/dT
SEQ ID NO: 8
mArAmArAmArUmUrUmCrAmCmCmCrUmUrAmArAmC/UNA-U/*/dT
SEQ ID NO: 9
UNA-G/*/mCrCmUrAmCrCmUrGrArGmGrAmCrAmGrUmArA/*/UNA-U/*/dT
SEQ ID NO: 10
mUrUmArCmUrGmUrCmCrUmCmAmGrGmUrAmGrGmC/UNA-U/*/dT
SEQ ID NO: 11
UNA-G/*/mArUmGrUmGrCmArCrArUmArUmCrAmGrGmArA/*/UNA-U/*/dT
SEQ ID NO: 12
mUrUmCrCmUrGmArUmArUmGmUmGrCmArCmArUmC/UNA-U/*/dT
SEQ ID NO: 13
GUUUAAGGGUGAAAUUUUU
SEQ ID NO: 14
AAAAAUUUCACCCUUAAAC
SEQ ID NO: 15
GCCUACCUGAGGACAGUAA
SEQ ID NO: 16
UUACUGUCCUCAGGUAGGC
SEQ ID NO: 17
GAUGUGCACAUAUCAGGAA
SEQ ID NO: 18
UUCCUGAUAUGUGCACAUC
SEQ ID NO: 19
GUUUAAGGGUGAAAUUUUUtt
SEQ ID NO: 20
AAAAAUUUCACCCUUAAACat
SEQ ID NO: 21
GCCUACCUGAGGACAGUAAtt
SEQ ID NO: 22
UUACUGUCCUCAGGUAGGCtc
SEQ ID NO: 23
GAUGUGCACAUAUCAGGAAtt
SEQ ID NO: 24
UUCCUGAUAUGUGCACAUCca
\hat{ } all sequences: 5' to 3';
For SEQ ID NOs:1-12: rN: ribonucleotides;
mN: 2'-O-methyl modified ribonucleotides;
UNA-N: unlocked nucleic acid base;
/*/: phosphorothioate (PS) bond;
dT: deoxy T; N: G, C, A, U, or T;
For SEQ ID NOs: 3-18: upper case—RNA; lower case—DNA

TABLE 4

| SEQ ID NO | Description |
|---|---|
| SEQ ID NO: 1 | UNA-si69108^{#} - sense |
| SEQ ID NO: 2 | UNA-si69108^{#} - antisense |
| SEQ ID NO: 3 | UNA-si69107^{#} - sense |
| SEQ ID NO: 4 | UNA-si69107^{#} - antisense |
| SEQ ID NO: 5 | UNA-si69109^{#} - sense |
| SEQ ID NO: 6 | UNA-si69109^{#} - antisense |
| SEQ ID NO: 7 | UNA-si69108^{#} - sense with 3′ dT |
| SEQ ID NO: 8 | UNA-si69108^{#} - antisense with 3′ dT |
| SEQ ID NO: 9 | UNA-si69107^{#} - sense with 3′ dT |
| SEQ ID NO: 10 | UNA-si69107^{#} - antisense with 3′ dT |
| SEQ ID NO: 11 | UNA-si69109^{#} - sense with 3′ dT |
| SEQ ID NO: 12 | UNA-si69109^{#} - antisense with 3′ dT |
| SEQ ID NO: 13 | si69108 - sense without DNA bases |
| SEQ ID NO: 14 | si69108 - antisense without DNA bases |
| SEQ ID NO: 15 | si69107 - sense without DNA bases |
| SEQ ID NO: 16 | si69107 - antisense without DNA bases |
| SEQ ID NO: 17 | si69109 - sense without DNA bases |
| SEQ ID NO: 18 | si69109 - antisense without DNA bases |
| SEQ ID NO: 19 | si69108 - sense |
| SEQ ID NO: 20 | si69108 - antisense |
| SEQ ID NO: 21 | si69107 - sense |
| SEQ ID NO: 22 | si69107 - antisense |
| SEQ ID NO: 23 | si69109 - sense |
| SEQ ID NO: 24 | si69109 - antisense |

^{#}chemically modified (CM) sequences including UNA monomers, as shown for sequences listed above

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20%, or ±10%, or ±5%, or even ±1% from the specified value, as such variations are appropriate for the disclosed methods or to perform the disclosed methods.

Any and all references and citations to other documents, such as patents, patent applications, patent publications, journals, books, papers, web contents, that have been made throughout this disclosure are hereby incorporated herein in their entirety for all purposes.

Although the invention has been described with reference to the above examples, it will be understood that modifications and variations are encompassed within the spirit and scope of the invention. Accordingly, the invention is limited only by the following claims.

1. A compound for RNA interference comprising:
(A)
(i) a sense strand comprising a sequence having at least 80% identity to the sequence of 5′-GUUUAAGGGUGAAAUUUUU-3′ (SEQ ID NO:13), and
(ii) an antisense strand comprising a sequence having at least 80% identity to the sequence of 5′-AAAAAUUUCACCCUUAAAC-3′ (SEQ ID NO:14); or
(B)
(i) a sense strand comprising a sequence having at least 80% identity to the sequence of 5′-GCCUACCUGAGGACAGUAA-3′ (SEQ ID NO:15), and
(ii) an antisense strand comprising a sequence having at least 80% identity to the sequence of 5′-UUACUGUCCUCAGGUAGGC-3′ (SEQ ID NO:16); or
(C)
(i) a sense strand comprising a sequence having at least 80% identity to the sequence of 5′-GAUGUGCACAUAUCAGGAA-3′ (SEQ ID NO:17), and
(ii) an antisense strand comprising a sequence having at least 80% identity to the sequence of 5′-UUCCUGAUAUGUGCACAUC-3′ (SEQ ID NO:18);
wherein the compound comprises at least one UNA monomer and at least one chemically modified monomer.

2. The compound of claim 1, wherein
G at a first position from the 5′ end of the sense strand is replaced by an UNA monomer.

3. The compound of claim 2, wherein
(a) the UNA monomer at the first position from the 5′ end of the sense strand is UNA-G, UNA-U, UNA-A, or UNA-C; or
(b) the UNA monomer at the first position from the 5′ end of the sense strand is UNA-G; or
(c) the UNA monomer at the first position from the 5′ end of the sense strand is connected to a 5′ unmodified monomer or a 5′ chemically modified monomer by a phosphorothioate linkage, a chiral phosphorothioate linkage, or a phosphorodithioate linkage; or
(d) (a) and (c); or
(e) (b) and (c).

4. (canceled)

5. (canceled)

6. The compound of claim 1, wherein the sense strand, the antisense strand, or both the sense strand and the antisense strand comprise an UNA monomer added at the 3′end.

7. The compound of claim 6, wherein
(a) the UNA monomer added at the 3′ end is UNA-U, UNA-G, UNA-A, UNA-C, or any combination thereof; or
(b) the UNA monomer added at the 3′ end is UNA-U; or
(c) the UNA monomer added at the 3′ end is connected to a 3′ unmodified monomer or a 3′ chemically modified monomer by a phosphorothioate linkage, a chiral phosphorothioate linkage, or a phosphorodithioate linkage; or
(d) (a) and (c); or
(e) (b) and (c).

8. (canceled)

9. (canceled)

10. The compound of claim 1, wherein the sense strand, the antisense strand, or both the sense strand and the antisense strand comprise at least one chemically modified monomer.

11. (canceled)

12. The compound of claim 1, wherein
(a) the at least one chemically modified monomer is a methylated monomer; or
(b) the at least one chemically modified monomer is a 2′-O-methyl modified monomer, an inverted thymidine monomer, an L-thymidine monomer, a glyceryl nucleotide, a 2′-methoxyethoxy nucleotide, a 2′-deoxy-2′-fluoro ribonucleotide, or any combination thereof.

13. (canceled)

14. The compound of claim 6,
(I) further comprising a deoxy T monomer connected to the UNA monomer added at the 3′end of the sense strand, the antisense strand, or both the sense strand and the antisense strand; or
(II) further comprising a deoxy T monomer connected to the UNA monomer added at the 3′end of the sense strand, the antisense strand, or both the sense strand and the antisense strand, wherein the deoxy T monomer is connected to the UNA monomer added at the 3′end of the sense strand, the antisense strand, or both the sense strand and the antisense strand by a phosphorothioate linkage, a chiral phosphorothioate linkage, or a phosphorodithioate linkage.

15. (canceled)

16. The compound of claim 1, comprising:
(I)
(i) a sense strand comprising a sequence having at least 80% identity to the sequence of SEQ ID NO:1, and
(ii) an antisense strand comprising a sequence having at least 80% identity to the sequence of SEQ ID NO:2; or
(II)
(i) a sense strand comprising a sequence having at least 80% identity to the sequence of SEQ ID NO:3, and
(ii) an antisense strand comprising a sequence having at least 80% identity to the sequence of SEQ ID NO:4; or
(III)
(i) a sense strand comprising a sequence having at least 80% identity to the sequence of SEQ ID NO:5, and
(ii) an antisense strand comprising a sequence having at least 80% identity to the sequence of SEQ ID NO:6.

17. The compound of claim 16,
(a) further comprising a deoxy T monomer at the 3′ end of the sense strand, the antisense strand, or both the sense strand and the antisense strand; or
(b) further comprising a deoxy T monomer at the 3′ end of the sense strand, the antisense strand, or both the sense strand and the antisense strand, wherein the deoxy T monomer is linked to the 3′ end of the sense strand, the antisense strand, or both the sense strand and the antisense strand by a phosphorothioate linkage, a chiral phosphorothioate linkage, or a phosphorodithioate linkage.

18. (canceled)

19. A compound for RNA interference, comprising:
(A)
(i) a sense strand comprising a sequence having at least 80% identity to the sequence of SEQ ID NO:7, and
(ii) an antisense strand comprising a sequence having at least 80% identity to the sequence of SEQ ID NO: 8; or
(B)
(i) a sense strand comprising a sequence having at least 80% identity to the sequence of SEQ ID NO:9, and
(ii) an antisense strand comprising a sequence having at least 80% identity to the sequence of SEQ ID NO:10; or
(C)
(i) a sense strand comprising a sequence having at least 80% identity to the sequence of SEQ ID NO:11, and
(ii) an antisense strand comprising a sequence having at least 80% identity to the sequence of SEQ ID NO:12.

20. The compound of claim 1, wherein the compound selectively inhibits lipopolysaccharide binding protein (LBP) expression.

21. A pharmaceutical composition comprising a compound of claim 1 and
(a) a pharmaceutically acceptable carrier; or
(b) a pharmaceutically acceptable carrier, wherein the carrier comprises a transfection reagent, a nanoparticle, or a liposome; or
(c) a pharmaceutically acceptable carrier, wherein the carrier comprises lipid nanoparticles; or
(d) a pharmaceutically acceptable carrier, wherein the carrier comprises lipid nanoparticles and wherein the lipid nanoparticles have a percent encapsulation of at least about 50%; or
(e) a pharmaceutically acceptable carrier, wherein the carrier comprises lipid nanoparticles having a size less than about 100 nm; or
(f) a pharmaceutically acceptable carrier, wherein the carrier comprises lipid nanoparticles having an average particle size of between about 50 and about 85 nm.

22. (canceled)

23. The pharmaceutical composition of claim 21, comprising a cationic lipid, a helper lipid, a cholesterol, and a PEG-lipid conjugate, wherein
(I) the cationic lipid is an ionizable cationic lipid; or
(II) the cationic lipid is an ionizable cationic lipid of Formula (V) 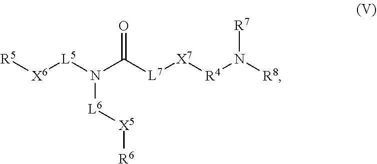
or a pharmaceutically acceptable salt or solvate thereof,
wherein R⁵ and R⁶ are each independently selected from the group consisting of a linear or branched C₁-C₃₁ alkyl, C₂-C₃₁ alkenyl or C₂-C₃₁ alkynl and cholesteryl;
L⁵ and L⁶ are each independently selected from the group consisting of a linear C₁-C₂₀ alkyl and C₂-C₂₀ alkenyl; X⁵ is —C(O)O— or —OC(O)—; X⁶ is —C(O)O—
or —OC(O)—; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl; or
(III) the cationic lipid is an ionizable cationic lipid having a structure selected from

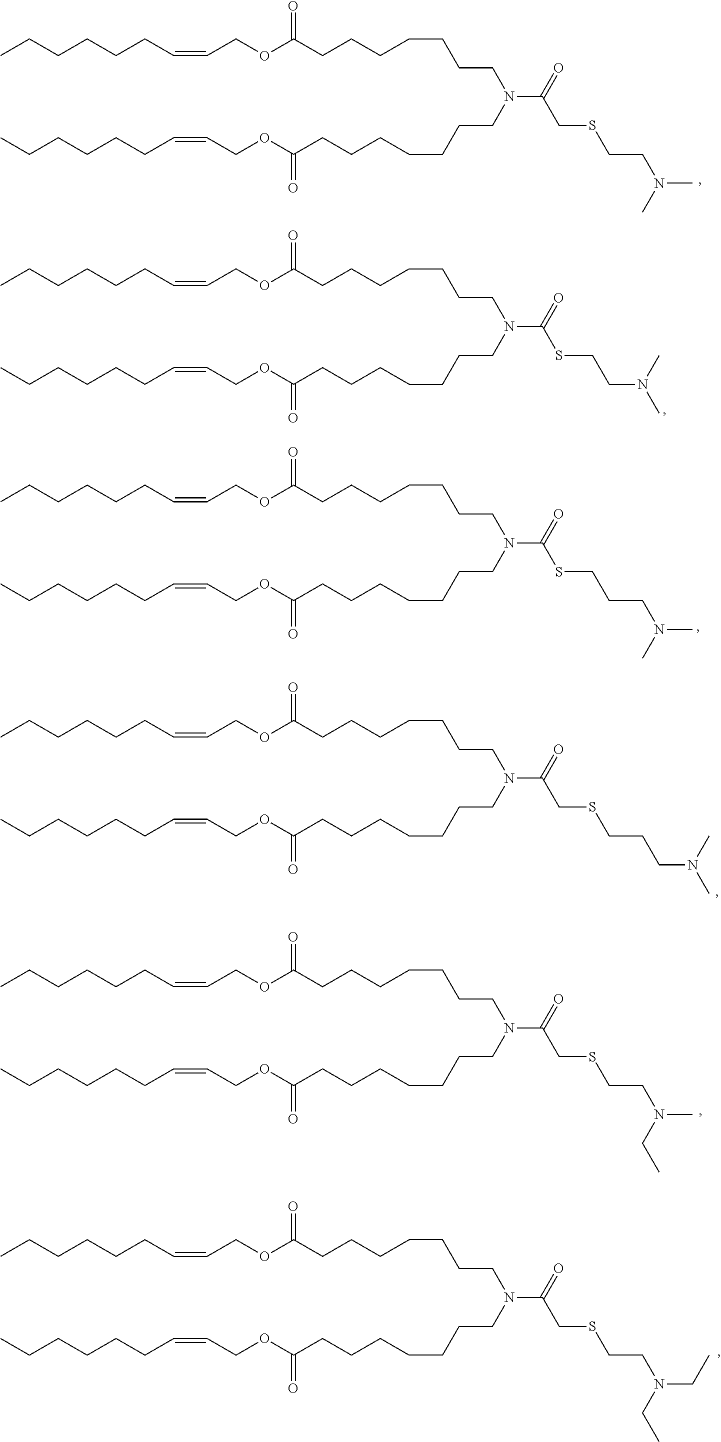

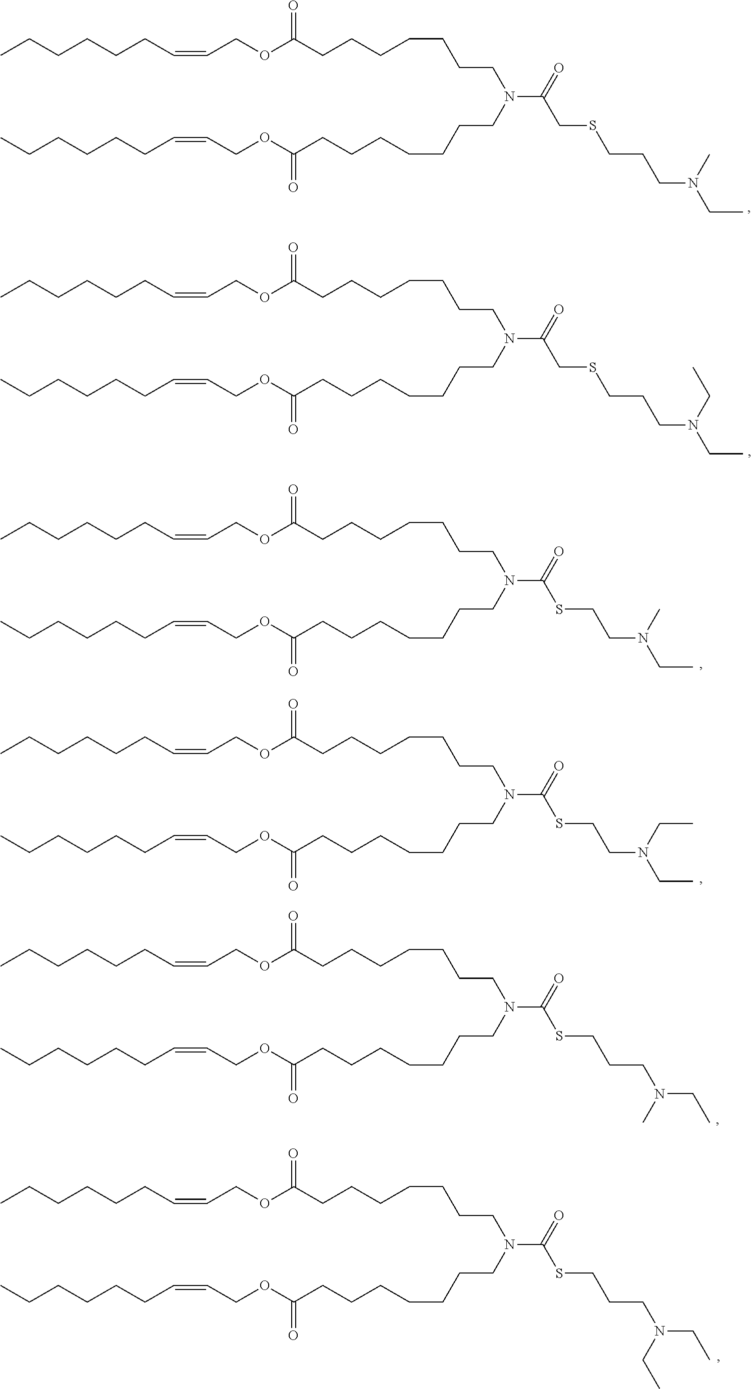

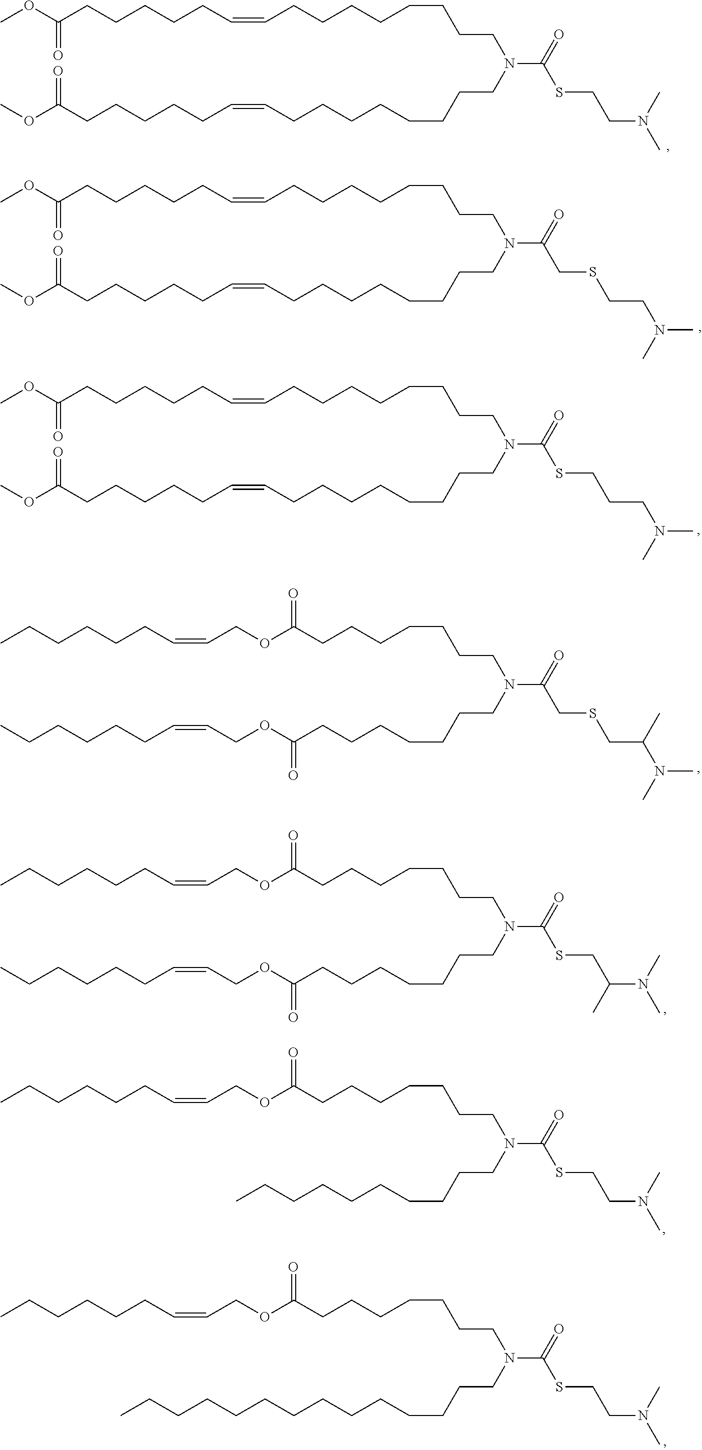

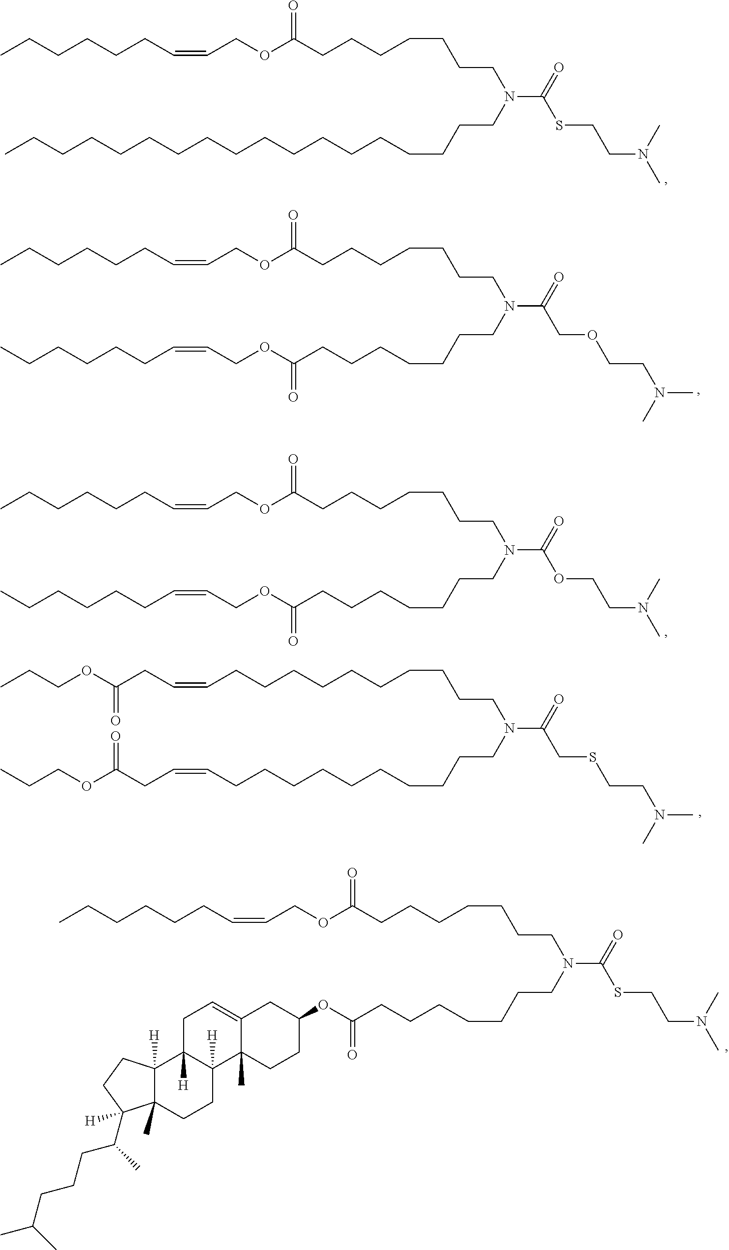

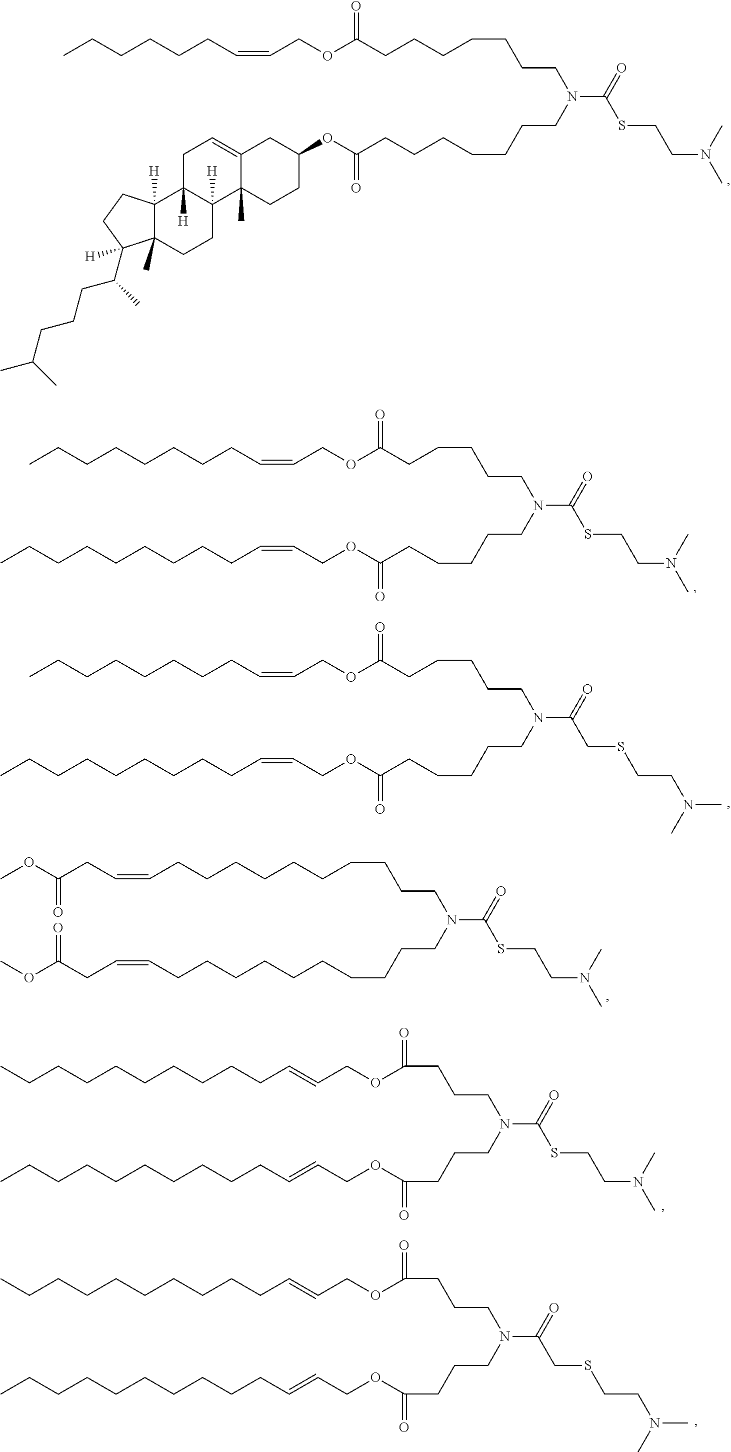

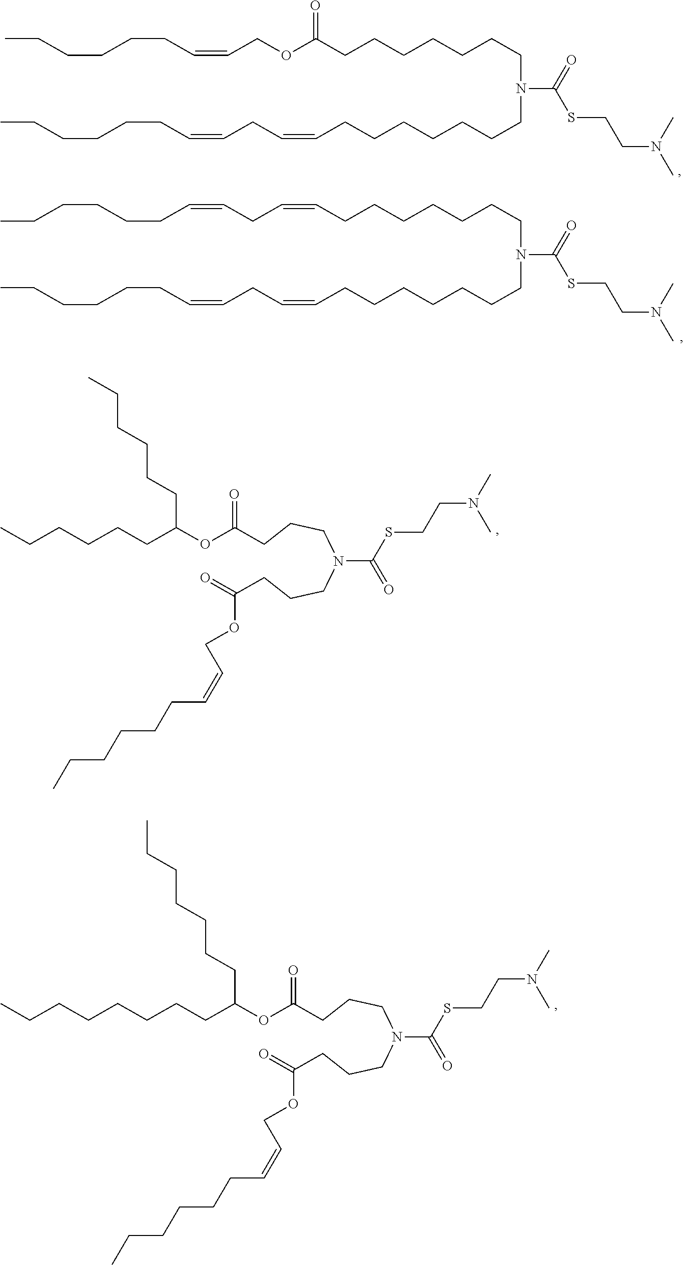

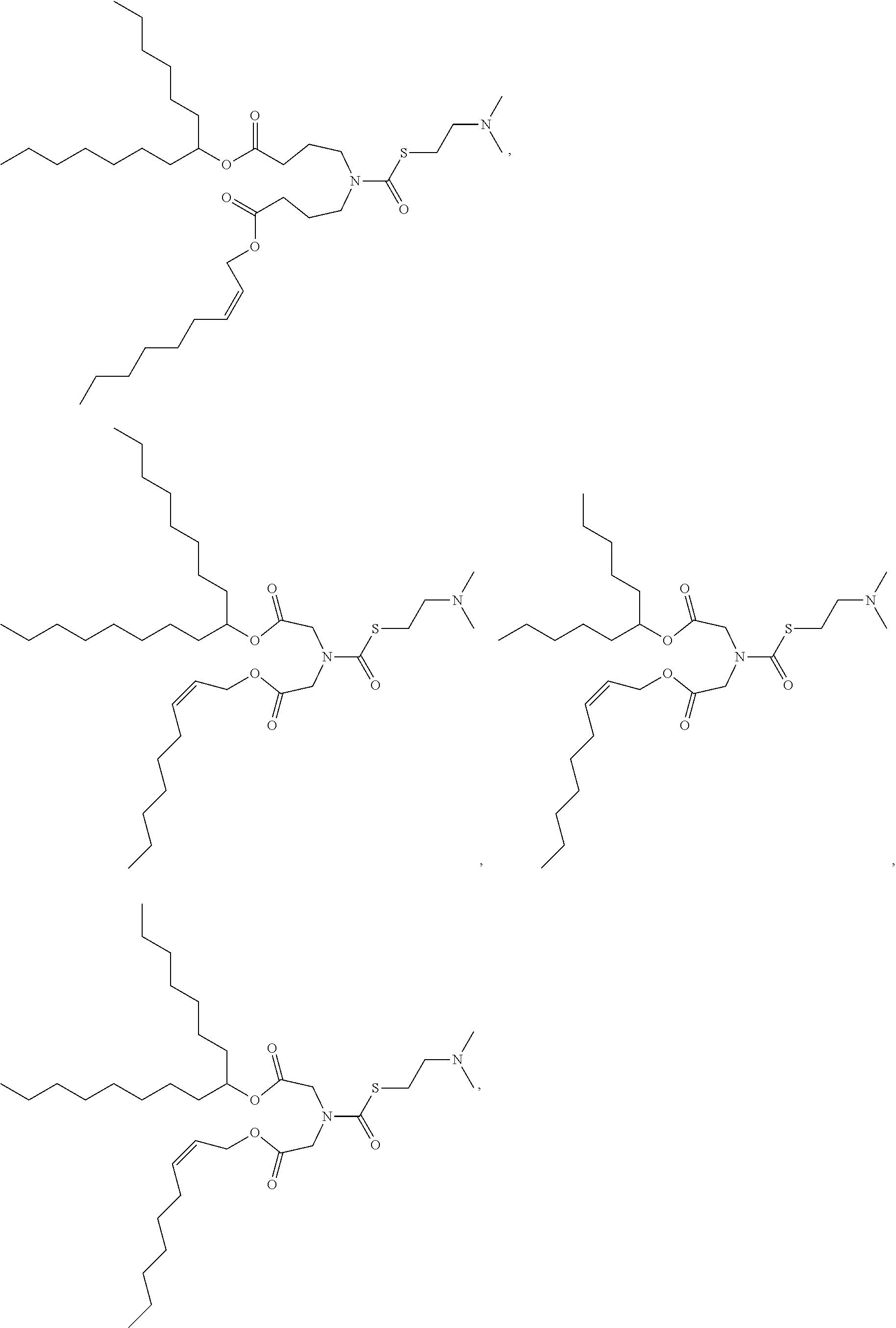

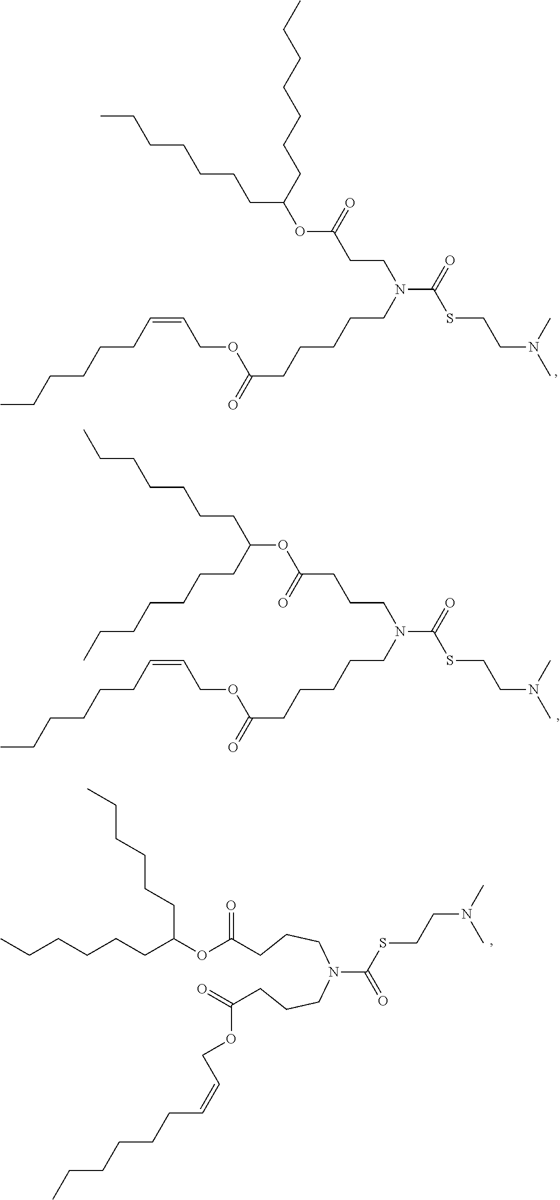

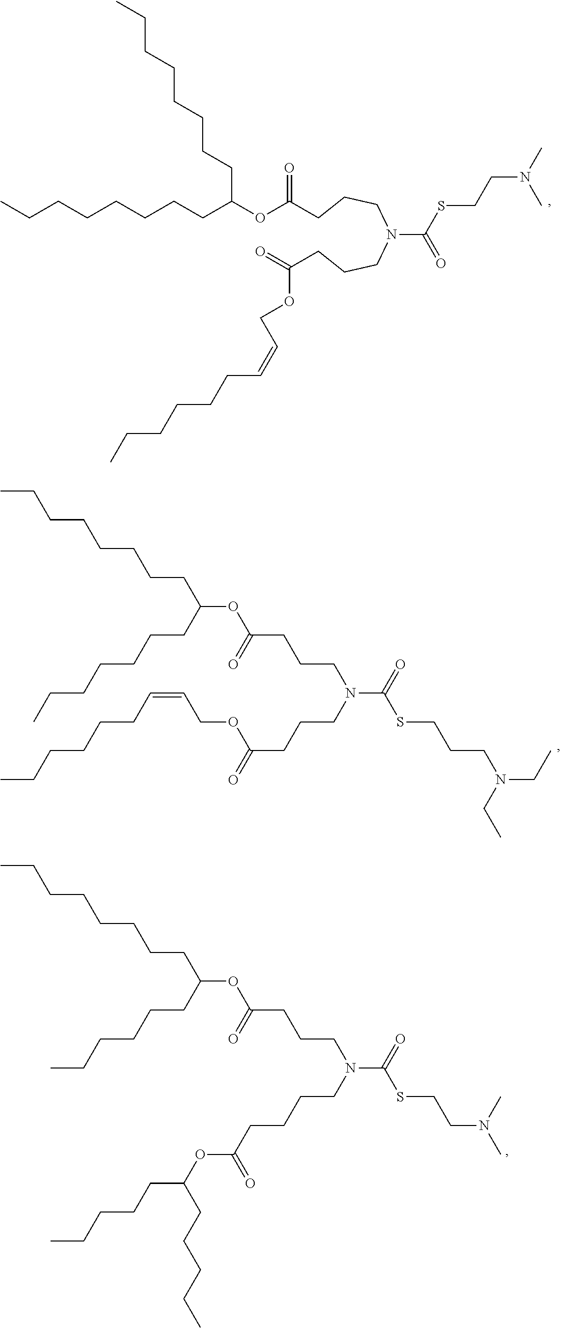

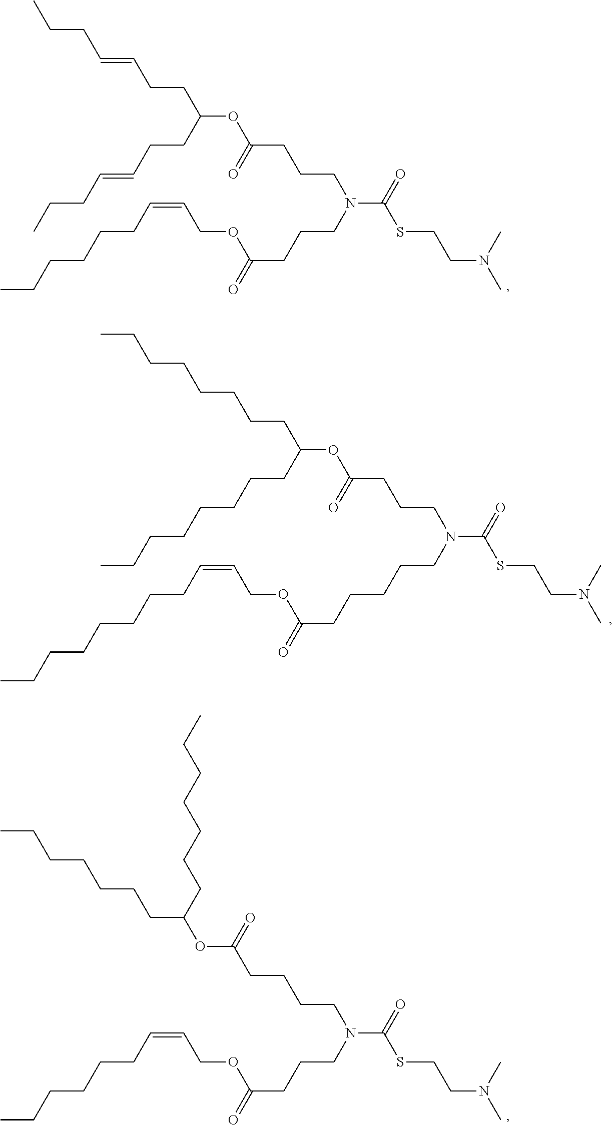

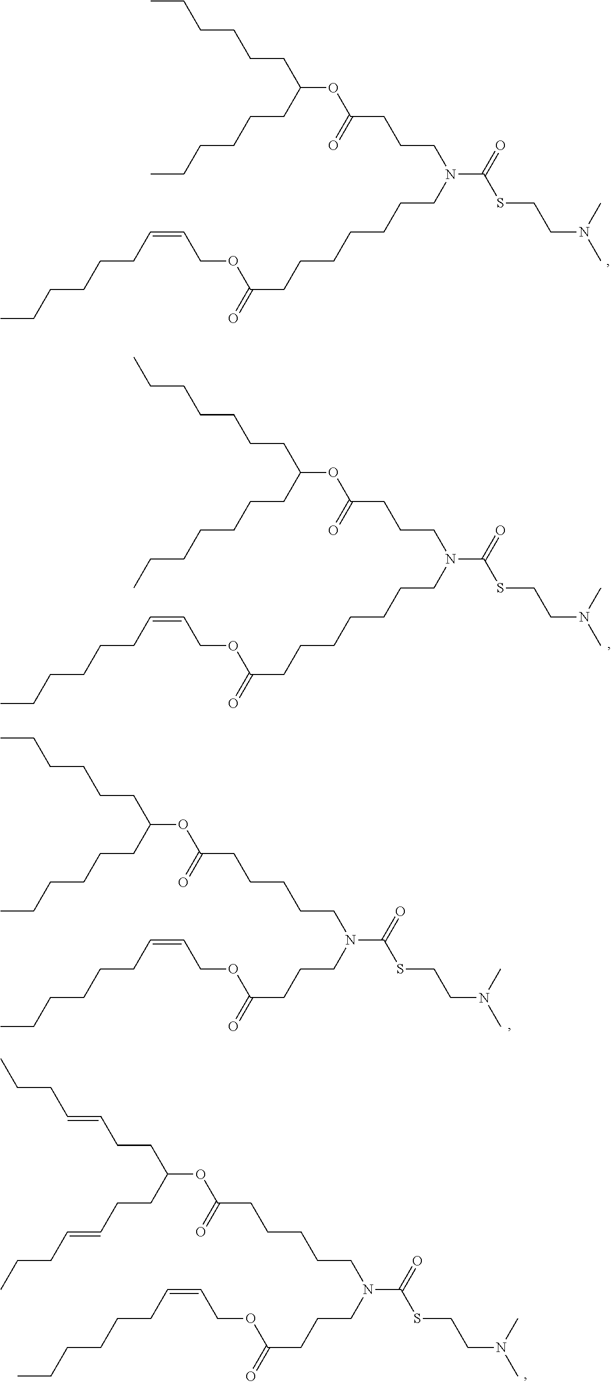

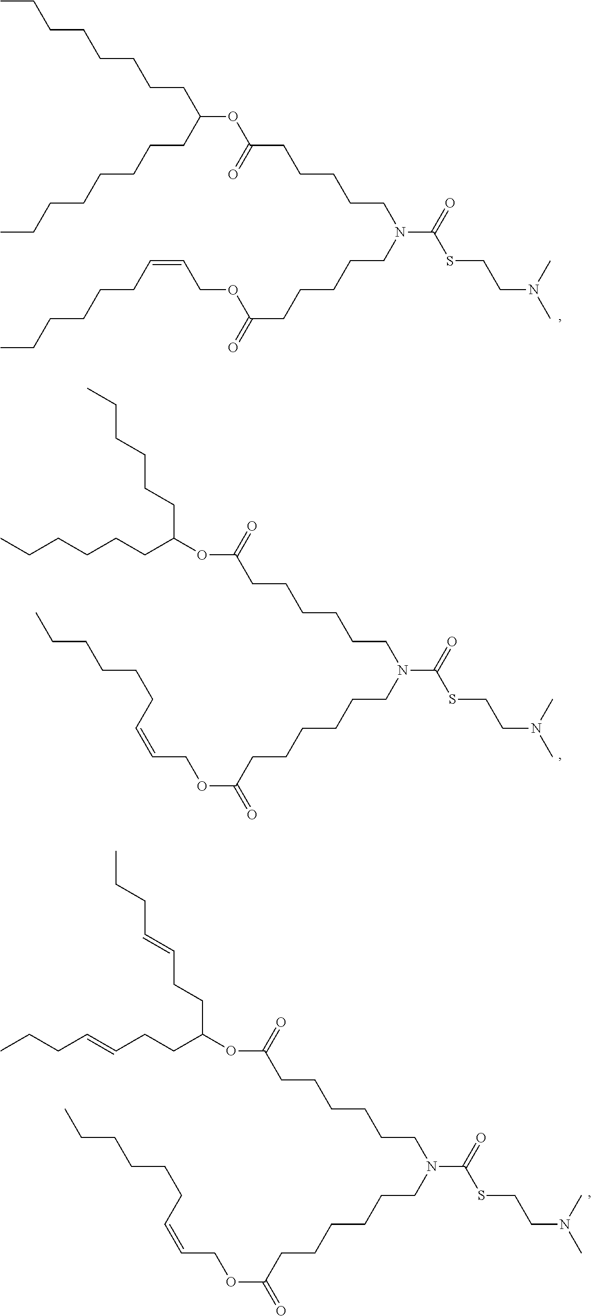

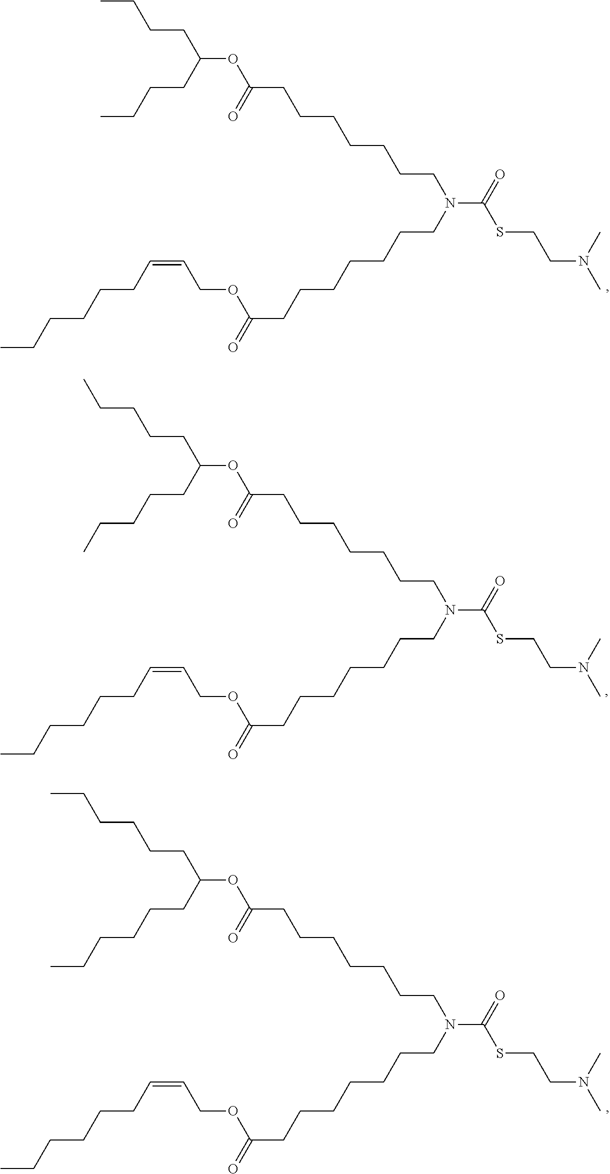

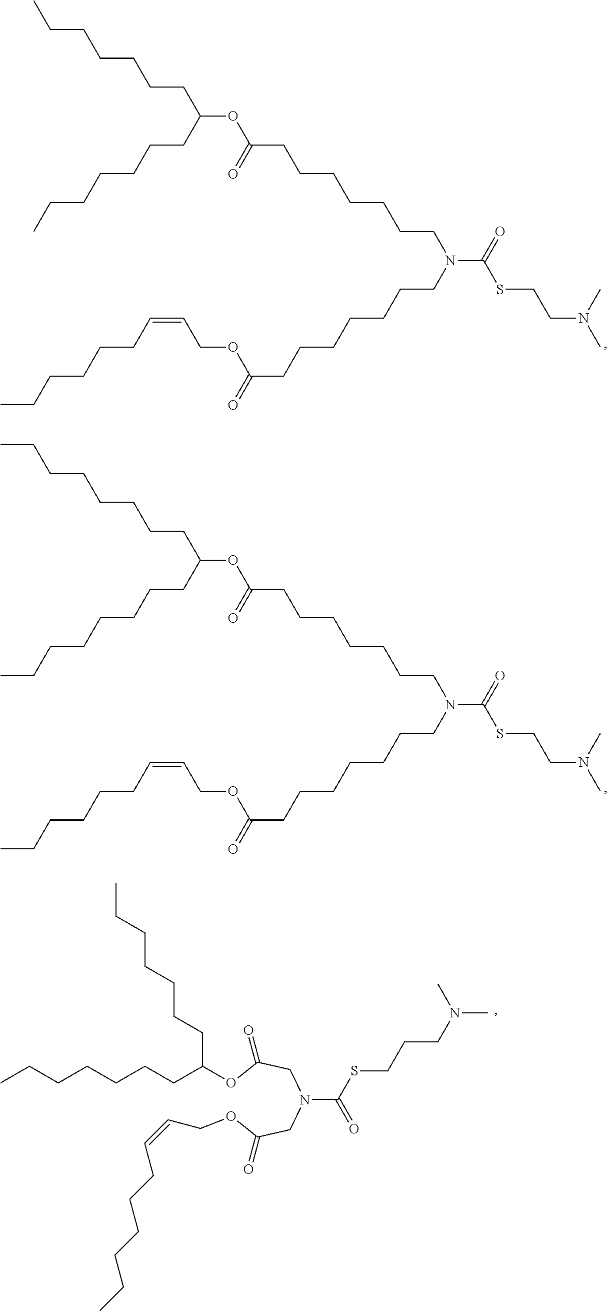

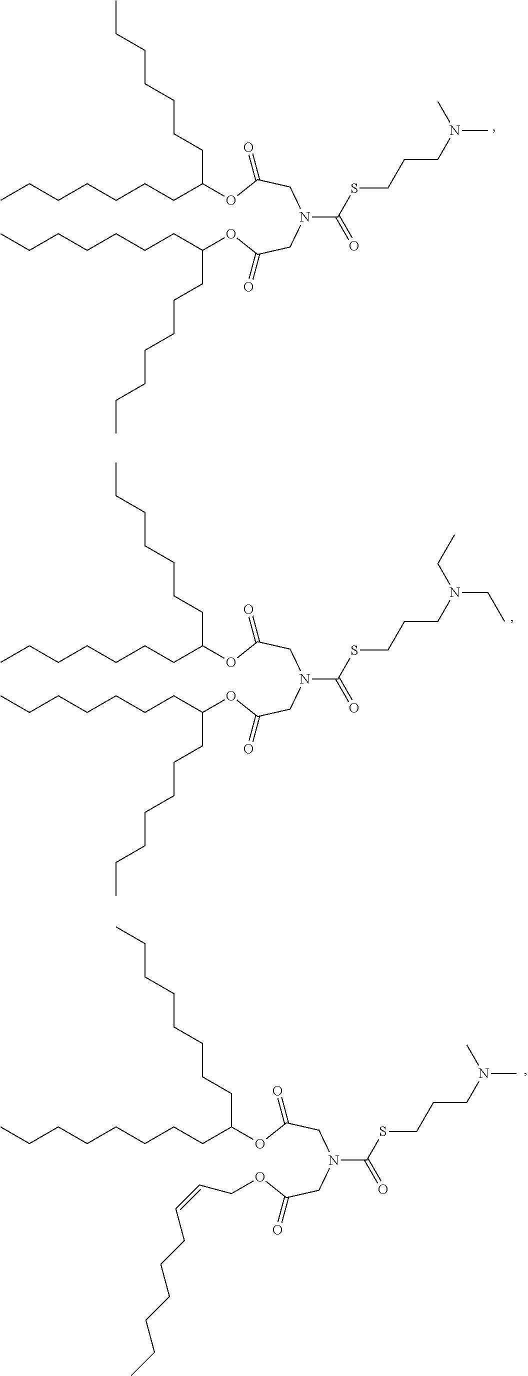

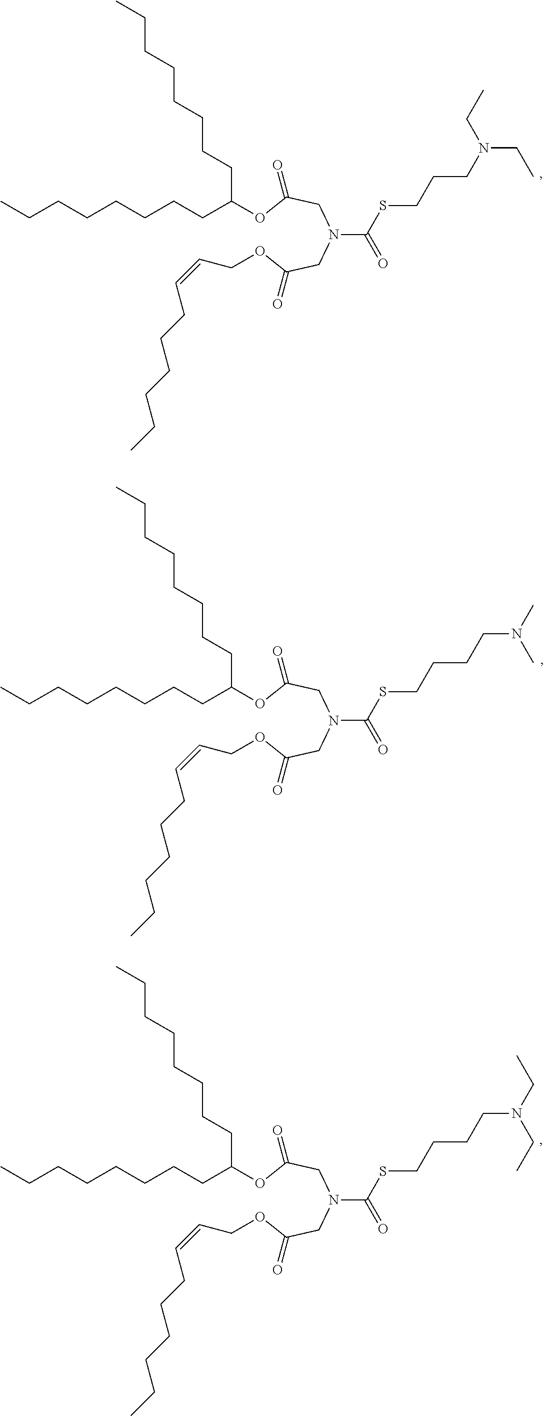

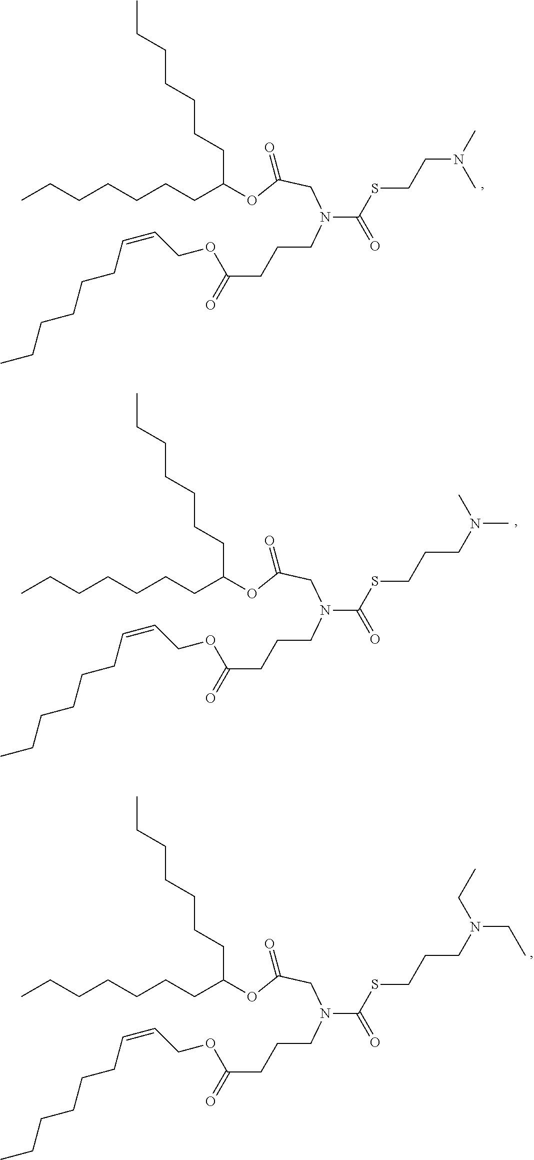

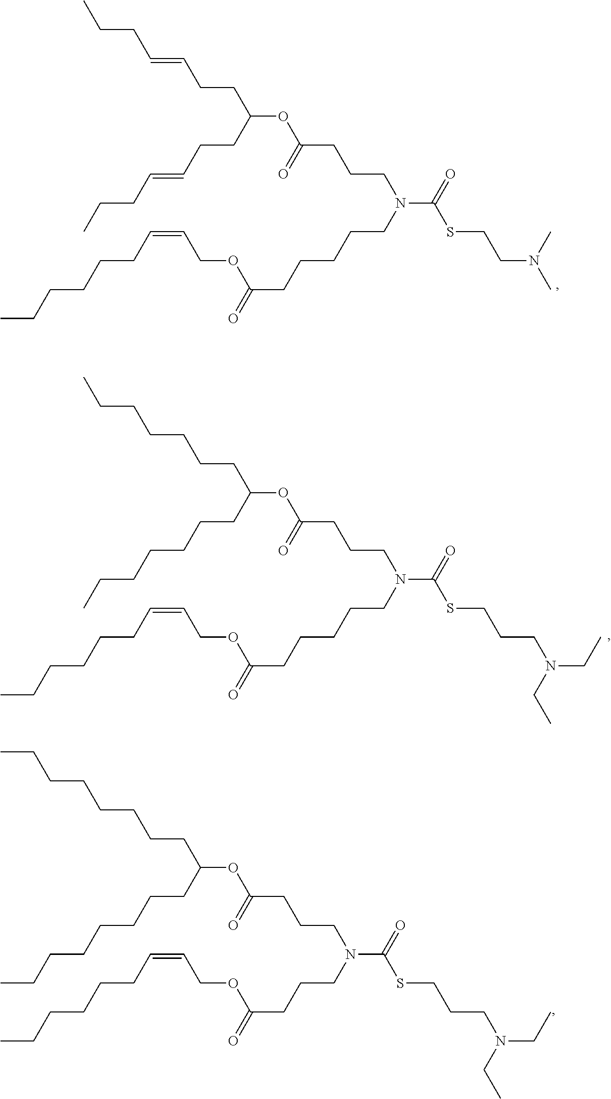

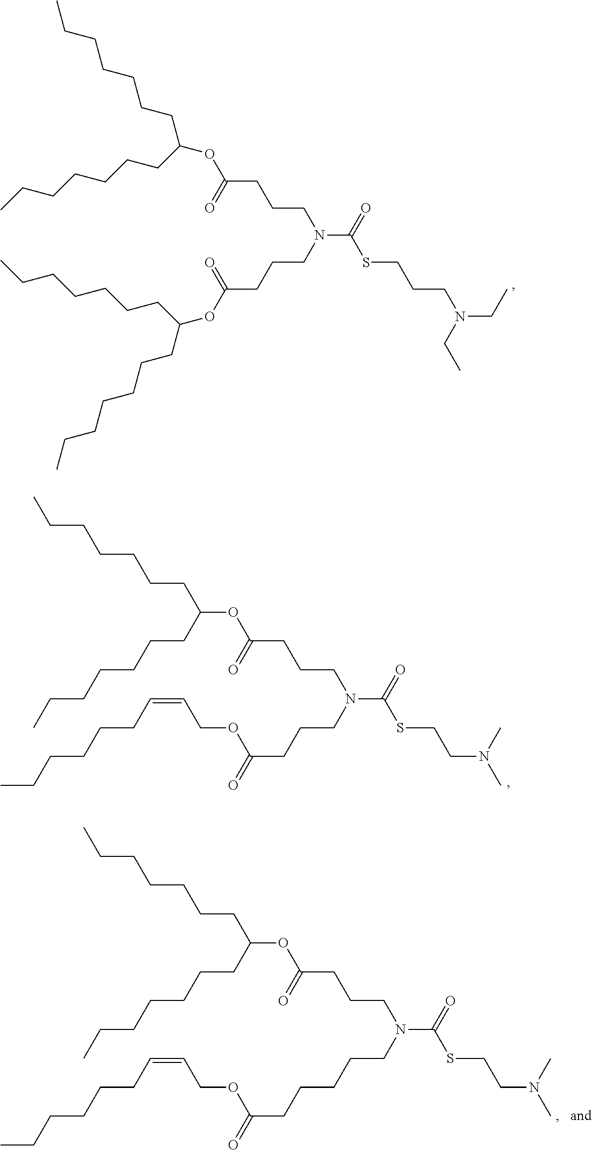

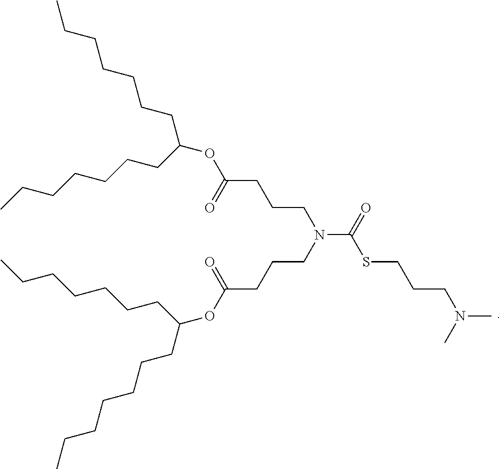 [SMILES: CCCCCCCC(CCCCCCC)OC(=O)CCCN(CCCC(=O)OC(CCCCCCC)CCCCCCC)C(=O)SCCCN(C)C]

24. (canceled)

25. (canceled)

26. (canceled)

27. (canceled)

28. (canceled)

29. (canceled)

30. The pharmaceutical composition of claim 23, wherein
(aa) the helper lipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), DOTAP, DOTMA, phosphatidylcholine (PC), or a combination of any of the foregoing; or
(bb) the helper lipid is distearoylphosphatidylcholine (DSPC); or
(cc) the PEG-lipid conjugate is PEG-DMG; or
(dd) PEG-DMG is PEG2000-DMG; or
(ee) the lipid nanoparticles comprise between about 20 mol % and 40 mol % of the cationic lipid, between about 25 mol % and 35 mol % of helper lipid, between about 25 mol % and 42 mol % cholesterol, and between about 0.5 mol % and 3 mol % PEG2000-DMG; or
(ff) the lipid nanoparticles comprise between about 20 mol % and 30 mol % of the cationic lipid, between about 30 mol % and 40 mol % of helper lipid, between about 34 mol % and 42 mol % cholesterol, and between about 1 mol % and 2 mol % PEG2000-DMG; or
(gg) the lipid nanoparticles comprise between about 22 mol % and 28 mol % of the cationic lipid, between about 31 mol % and 39 mol % of helper lipid, between about 35 mol % and 40 mol % cholesterol, and between about 1.25 mol % and 1.75 mol % PEG2000-DMG; or
(hh) the pharmaceutical composition has a total lipid:compound weight ratio of between about 8:1 and 40:1; or
(ii) the pharmaceutical composition comprises between about 20 w/w % and 60 w/w % of the cationic lipid; or
(jj) any combination thereof.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A method of treating or preventing a metabolic disorder or condition in a subject comprising:
administering to the subject an amount of the pharmaceutical composition of claim 21 effective for treating or preventing the metabolic disorder or condition.

43. The method of claim 42, wherein
(I) the metabolic disorder or condition is obesity, liver steatosis, obesity-associated liver steatosis, liver inflammation, liver fibrosis, cardiovascular disease, atherosclerosis, coronary artery disease, diabetes, prediabetes, obesity-associated fat accretion, or obesity-related adipose tissue dysfunction; or
(II) the metabolic disorder or condition is an LBP-mediated disorder; or
(III) the administering decreases liver lipid accumulation, decreases liver triglyceride levels, decreases lipogenic and oxidative stress gene expression, decreases insulin resistance, reduces glycemia, or any combination thereof, as compared with administering a control compound or vehicle; or
(IV) the administering increases food consumption, reduces fat mass gain, increases circulating adiponectin levels, increases expression of adipogenic and thermogenic genes in adipose tissue, or any combination thereof, as compared with administering a control compound or vehicle; or
(V) the administering increases food consumption, reduces fat mass gain, increases expression of adipogenic and thermogenic genes in adipose tissue, or any combination thereof, as compared with administering a control compound or vehicle; or
(VI) any combination thereof.

44. (canceled)

45. (canceled)

46. (canceled)

47. The method of claim 43, wherein increased food consumption, reduced fat mass gain, and increased expression of adipogenic and thermogenic genes in adipose tissue is sex-specific or female-specific.

48. (canceled)

49. (canceled)

50. The method of claim 42, wherein the pharmaceutical composition is administered intravenously, orally, sublingually, intramuscularly, subcutaneously, intradermally, transdermally, intranasally, intraperitoneally, topically, or by a pulmonary route.

51. The method of claim 42, wherein
(aa) the subject suffers from the metabolic disorder or condition, the subject is a healthy subject, the subject is an obese subject, or the subject is a non-obese subject; and/or
(bb) the subject consumes a high fat diet.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)
